SEQUENCE LISTING (14 sequences)

SEQ ID NO: 1 - DNA, 3054 bp, rattus norvegicus
  CDS (86)...(1576)
tcagagttccgcgcagaaccttccagtgcccagccatacttcaggagtgccctgtccccttagtcgtggctgcaggcgtggagccatgagctgtcttctgaacaatatggtcccggtggggctggctctgctggtctgcggagtacaggcctttttccttcccaacaccacgagcctggagaggctgctcagcaagtaccagcacgcagagccacactcacgcgtgcgcagggccatccccatgtcagaccgtcaggaaatcctcatgctgcacaacaagctgcgaggtcaggtgtatccccctgcctccaacatggagtacatgacctgggatgaggagctggagaggtcggcagccgcgtgggcacagaggtgcctgtgggagcatgggcccgccagccttctcgtgtccatcgggcagaacctggctgtgcactggggcaggtaccgctctcctgggttccacgtgcagtcgtggtatgacgaggtgaaggattacacctacccgtacccccacgagtgcaacccatggtgccccgagcgctgctcaggagccatgtgcacccactacacacagatggtttgggccaccaccaacaagattggctgtgctgtgcacacctgtcggagcatgagcgtctggggagacatctgggagaacgccgtgtatctcgtctgcaattattctccaaagggaaactggattggtgaggctccctacaaacatggccgcccctgctctgagtgtccgtccagctatggaggtggctgccggaacaacctgtgttaccgagaagagcactaccatcagaaaccggaggtggatgagatgaacgaggtggaatcaccccctgctcccgaggaaacccatgtctgggttcaacccagggtggtcaaaccctcaaagaccaagaagaccccagtcgtcaacttcatgacccaagtggtccactgtgacacaaagatgaaggactcatgcaagggatccacatgtaacaggtaccagtgcccagcaggctgtcggaacaacaaggcgaaggtctttggctctctgttttatgaaagttcttccagtatatgtcgagctgctatccactatggtgtcatcgatgatcgaggtggcctggtggatgtcaccaggaatgggatggtacccttctttgtcaaatctcagaaaaatggcttggagtccctgagtaaatacaagccgtctagctccttcactgtgtccaaagtgaaagagacggctgtggactgccacaccacagttgcacagctatgccccttcgagaagccagccacccactgcccgagagtccggtgtccttcacgatgtggagaggagccatcctattgggcccctgtgtatggaaccaacatctacgctgacacttccagcatctgtaaggcggctgtgcatgcaggcgtcattgtcgacgaggttggtggctatgcagatgtgatgcccgtggacaaaaagaagagctacgtgggctccctcaggaacggggttcagtcagagagcccgagcactcctcagaacggaaacgccttccggatcttccccgtcaggcagtgaatatggagacctggggagaagggggtgtcgtctactgggattcgggggtcctgctttttatctgtactttgtcctttgggggtgggagggttacagagagtcaagaatcttcctttcggtgtcgttgactgtcctgggcctccgtggcttttggtgacggtgacagagcaccttcccctcgtgagcagcatgccgtggagtctcttgctggacaaccagtgcccagactggccaggtggcggtccccatctggtggctcatctcctctcaggagatgctcaccgagacactctttgccacattttctttggtgttggaggggggaaggctttcgaactcttgaggatacagggtggagattcgttgtgaagggggctcttgtctggatgacaagagtggacagccctaaggttacagtccgtcctaccaacagagaaatagggtctgtgtttgctggtcagacagatgggctgcagggagaagccctgggcaaaacctgggctctgccctggtggtatccgtgctcatgtccccaaccttcttggcagtcacgtgttcgtatgaccagcagagctggtctatttaaagttggtagcactccagagatttatcttttctttgtccttgtccttgtccttgtggcccgttttcacatgacctttggtctcatggaagaccagtgcatcaggagccttccccaccattccgtgtccctgtgtcgagcatctgccccaccccaaggaggtctagattgtcctcctttggggcttggctttgtaagaggctccaggtcttagatgtctgcttactccttagaacaagggctctctcacggttgctgttagagtggcagctcgacatcgcttccccatggtttgggtgacctagtttttagctgtgggaggaccggattcctggcgcattcctcctgggggttaatggagtgacctgtggttgtaaaaccacccatgacacaccagccaccagagagagaggggcagggacacacaggaagaacccctcagcgcttgccagagtcccgggctccaagagtgtggtgagtcctggcttccatcacttaggagcctggtccaaacccgctgtaaaatgaaacctgcttttgtaaacagctttctctagagaaggtggaatcccaccccagtcctggggatggggcacagttgcctgtgaatgttggcctcaaacagggtttcatgatccagccaaacatttcttagacaaaagaaagggggtgggagtagaaaataaaacagaatgctgtcccccagttgaaaggctggggaggtgacttggtgattaacagctctaccagaggacctgagctcagttcccacgactcacagactataactataacccacctatgaccaactctaggagatccatgatctccacagacgccacacgcatccacatgagcaaaagtacaaatggtcctaaaaaaaaaaaaaaaa

SEQ ID NO: 2 - protein, 497 aa, rattus norvegicus
MSCLLNNMVPVGLALLVCGVQAFFLPNTTSLERLLSKYQHAEPHSRVRRAIPMSDRQEILMLHNKLRGQVYPPASNMEYMTWDEELERSAAAWAQRCLWEHGPASLLVSIGQNLAVHWGRYRSPGFHVQSWYDEVKDYTYPYPHECNPWCPERCSGAMCTHYTQMVWATTNKIGCAVHTCRSMSVWGDIWENAVYLVCNYSPKGNWIGEAPYKHGRPCSECPSSYGGGCRNNLCYREEHYHQKPEVDEMNEVESPPAPEETHVWVQPRVVKPSKTKKTPVVNFMTQVVHCDTKMKDSCKGSTCNRYQCPAGCRNNKAKVFGSLFYESSSSICRAAIHYGVIDDRGGLVDVTRNGMVPFFVKSQKNGLESLSKYKPSSSFTVSKVKETAVDCHTTVAQLCPFEKPATHCPRVRCPSRCGEEPSYWAPVYGTNIYADTSSICKAAVHAGVIVDEVGGYADVMPVDKKKSYVGSLRNGVQSESPSTPQNGNAFRIFPVRQ

SEQ ID NO: 3 - DNA, 2896 bp, Homo sapiens
  CDS (32)...(1525)
tccccttattcgtggctgcaggcgtggagccatgagctgtcttctgaacaatatggtcccggtggggctggctccgctggtctgcggagtacaggcctttttccttcccaacaccatgagcctggagaggctgctcagcaagtaccagcacacagagccacactcacgcgtgcgcagggccatccccatgtcagaccgtcaggaaatcctcatgctgcacaacaagctgcgaggtcaggtgtatccccctgcctccaacatggagtacatgacctgggatgaggagctggagaggtcggcagccgcgtgggcacagaggtgcctgtgggagcatgggcccgccagccttctcgtgtccatcgggcagaacctggctgtgcactggggcaggtaccgctctcctgggttccacgtgcagtcgtggtatgacgaggtgaaggattacacctacccgtacccccacgagtgcaacccatggtgccccgagcgctgctcaggagccatgtgcacccactacacacagatggtttgggccaccaccaacaagattggctgtgctgtgcacacctgtcggagcatgagcgtctggggagacatctgggagaacgccgtgtatctcgtctgcaattattctccaaagggaaactggattggtgaggctccctacaaacatggccgcccctgctctgagtgtccgtccagctatggaggtggctgccggaacaacctgtgttaccgagaagagcactaccatcagaaaccggaggtggatgagatgaacgaggtggaatcaccccctgctcccgaggaaacccatgtctgggttcaacccagggtggtcaaaccctcaaagaccaagaagaccccagtcgtcaacttcatgacccaagtggtccactgtgacacaaagatgaaggactcatgcaagggatccacatgtaacaggtaccagtgcccagcaggctgtctgaacaacaaggcgaaggtctttggctctctgttttatgaaagttcttccagtatatgtcgagctgctatccactatggtgtcatcgatgatcgaggtggcctggtggatgtcaccaggaatgggatggtacccttctttgtcaaatctcagaaaaatggcttgaagtccctgagtaaatacaagccgtctagctccttcactgtgtccaaagtgaaagagacggctgtggactgccacaccacagttgcacagctatgccccttcgagaagccagccacccactgcccgagagtccggtgtccttcacgatgtggagaggagccatcctattgggcccctgtgtatggaaccaacatctacgctgacacttccagcatctgtaaggcggctgtgcatgcaggcgtcattgtcgacgaggttggtggctatgcagatgtgatgcccgtggacaaaaagaagagctacgtgggctccctcaggaacggggttcagtcagagagcccgagcactcctcagaacggaaacgccttccggatcttccccgtcaggcagtgaatatggagacctggggagaagggggtgtcgtctactgggattcgggggtcctgctttttatctgtactttgtcctttgggggtgggagggttacagagagtcaagaatcttcctttcggtgtcgttgactgtcctgggcctccgtggcttttggtgacggtgacagagcaccttcccctcgtgagcagcatgccgtggagtctcttgctggacaaccagtgcccagactggccaggtggcggtccccatctggtggctcatctcctctcaggagatgctcaccgagacactctttgccacattttctttggtgttggaggggggaaggctttcgaactcttgaggatacagggttgagattcgttgtgaagggggctcttgtctggatgacaagagtggacagccctaaggttacagtccgtcctaccaacagagaaatagggtctgtgtttgctggtcagacagatgggctgcagggagaagccctgggcaaaacctgggctctgccctggtggtatccgtgctcatgtccccaaccttcttggcagtcacgtgttcgtatgaccagcagagctggtctatttaaagttggtagcactccagagatttatcttttctttgtccttgtccttgtccttgtggcccgttttcacatgacctttggtctcatggaagaccagtgcatcaggagccttccccaccattccgtgtccctgtgtcgagcatctgccccaccccaaggaggtctagattgtcctcctttggggcttggctttgtaagaggctccaggtcttagatgtctgcttactccttagaacaagggctctctcacggttgctgttagagtggcagctcgacatcgcttccccatggtttgggtgacctagtttttagctgtgggaggaccggattcctggcgcattcctcctgggggttaatggagtgacctgtggttgtaaaaccacccatgacacaccagccaccagagagagaggggcagggacacacaggaagaacccctcagcgcttgccagagtcccgggctccaagagtgtggtgagtcctggcttccatcacttaggagcctggtccaaacccgctgtaaaatgaaacctgcttttgtaaacagctttctctagagaaggtggaatcccaccccagtcctggggatggggcacagttgcctgtgaatgttggcctcaaacagggtttcatgatccagccaaacatttcttagacaaaagaaagggggtgggagtagaaaataaaacagaatgctgtcccccagttgaaaggctggggaggtgacttggtgattaacagctctaccagaggacctgagctcagttcccacgactcacagactataa

SEQ ID NO: 4 - protein, 497 aa, homo sapiens
MSCLLNNMVPVGLAPLVCGVQAFFLPNTMSLERLLSKYQHTEPHSRVRRAIPMSDRQEILMLHNKLRGQVYPPASNMEYMTWDEELERSAAAWAQRCLWEHGPASLLVSIGQNLAVHWGRYRSPGFHVQSWYDEVKDYTYPYPHECNPWCPERCSGAMCTHYTQMVWATTNKIGCAVHTCRSMSVWGDIWENAVYLVCNYSPKGNWIGEAPYKHGRPCSECPSSYGGGCRNNLCYREEHYHQKPEVDEMNEVESPPAPEETHVWVQPRVVKPSKTKKTPVVNFMTQVVHCDTKMKDSCKGSTCNRYQCPAGCLNNKAKVFGSLFYESSSSICRAAIHYGVIDDRGGLVDVTRNGMVPFFVKSQKNGLKSLSKYKPSSSFTVSKVKETAVDCHTTVAQLCPFEKPATHCPRVRCPSRCGEEPSYWAPVYGTNIYADTSSICKAAVHAGVIVDEVGGYADVMPVDKKKSYVGSLRNGVQSESPSTPQNGNAFRIFPVRQ

SEQ ID NO: 5 - DNA, 21 bp, Artificial Sequence
  PCR Primer
atgctgcacaacaaggctgcg

SEQ ID NO: 6 - DNA, 21 bp, Artificial Sequence
  PCR Primer
gctctgagtgtccgtccagct

SEQ ID NO: 7 - DNA, 21 bp, Artificial Sequence
  PCR Primer
gtgggccgctctaggcaccaa

SEQ ID NO: 8 - DNA, 24 bp, Artificial Sequence
  PCR Primer
ctctttgatgtcacgcagcatttc

SEQ ID NO: 9 - DNA, 18 bp, Artificial Sequence
  antisense
gttgtgcagcatgaggat

SEQ ID NO: 10 - DNA, 18 bp, Artificial Sequence
  control sense sequence
atcctcatgctgcacaac

SEQ ID NO: 11 - DNA, 18 bp, Artificial Sequence
  Control srambled sequence
gtacttgacgcttgagaa

SEQ ID NO: 12 - DNA, 82 bp, Homo sapiens
  CDS (1)...(81)
atgctgcacaacaagctgcgaggtcaggtgtatccccctgcctccaacatggagtacatgacctgggatgaggagctggaga

SEQ ID NO: 13 - protein, 27 aa, Homo sapiens
MLHNKLRGQVYPPASNMEYMTWDEELE

SEQ ID NO: 14 - protein, 17 aa, rattus norvegicus
CVREPEHSSERKRFRIF

(19) United States
(12) Patent Application Publication
(10) Pub. No.: US 2005/0119459 A1
(43) Pub. Date: Jun. 2, 2005

(54) Late gestation lung genes, fragments and uses thereof

(76) Inventors: Feige Kaplan, Hampstead (CA); Neil B. Sweezey, Aurora (CA)

Correspondence Address: Madeline I Johnson;King & Spalding, 45th Floor, 191 Peachtree Street, Atlanta, GA, 30303, US

(21) Appl. No.: 10/488,528

(22) PCT Filed: Sep. 4, 2002
(86) PCT No.: PCT/CA02/01350

Related U.S. Application Data
(60) Provisional application No. 60/336,598, filed on Dec. 5, 2001.

(30) Foreign Application Priority Data
Sep. 4, 2001 (CA) 2,357,746

(51) Int. Cl. C12Q 1/68; C07H 21/04; C07K 14/785

(52) U.S. Cl. 530/350; 435/6; 435/69.1; 435/320.1; 435/325; 514/12; 536/23.5

(57) ABSTRACT

The present invention provides a family of genes related to late gestation lung genes and fragments thereof. Embodiments of the present invention provide compositions and methods for the therapeutic treatment of disorders in the lung or other tissues. More particularly the invention provides methods for the treatment of abnormalities in alveolarization, and abnormalities in branching morphogenesis. In other embodiments of the invention the use of the LGL1 gene or related products or fragments thereof in research and diagnostics is provided.

### FIELD OF THE INVENTION

The present invention relates to late gestation lung genes and fragments thereof, for use in research, diagnostics and the preparation of therapeutics to treat disease.

### BACKGROUND

Lung Disease

In the mature lung, defects in alveolarization, whether related to a primary failure of alveolar formation or to inadequate tissue repair in disease, form the major burden in various pulmonary conditions. These include emphysema, chronic obstructive pulmonary disease (COPD), congenital diaphragmatic hernia (CDH), bronchopulmonary dysplasia (BPD) and New BPD. Collectively, these conditions are responsible for significant annual morbidity and mortality in developing countries and in the industrialized world.

Chronic obstructive pulmonary disease (COPD), of which emphysema and chronic bronchitis comprise the principal subsets, is a major lung disorder which usually presents clinically in adulthood. In the US alone, the patient population having COPD is approximately 17 million people. Although there are multiple risk factors associated with COPD, it is well recognized that there is a genetic predisposition to emphysema; 75,000-100,000 people in the US have a genetic deficiency of alpha 1-antitrypsin, the lack or deficiency of which is a chief causative risk factor. Cigarette smoke toxicity has also clearly been implicated as a causative factor. Emphysema is characterized by progressive, marked destruction of the inter-alveolar septae, leading to reduced alveolar number, lung hyperinflation and loss of the elastic recoil of the lung parenchyma. Pulmonary oxygen diffusion becomes compromised as the effective gas exchange surface area is progressively reduced. Current therapies for COPD and emphysema (e.g. bronchodilators, anticholinergics, steroids, oxygen therapy, and aerosolized surfactant) merely address the secondary manifestations of disease; there is a need for therapies that directly address the inadequacy of gas exchange units (alveoli) in the lung.

In congenital diaphragmatic hernia (CDH), ipsilateral lung growth and alveolar number are reduced, providing a clinical model of lung hypoplasia To the extent that compensatory growth and alveolar formation occurs after surgical correction of the hernia with reduction of the intestines back into the abdominal cavity, these patients can do very well from a pulmonary point of view. This supports the hypothesis that the induction of alveolar formation, if carried out to a sufficient extent, can make a clinically significant difference.

Pulmonary disorders for which there is no satisfactory therapeutic regimen represent an important medical challenge and need. Although the etiologies of the specific disease states listed above remain multifactorial, they arise in large part from abnormal lung development. Deficiency of lung maturation due to premature birth is a major cause of morbidity and mortality in the newborn with infant respiratory distress syndrome. Sequelae of respiratory distress syndrome can often extend into childhood and into adulthood as the chronic respiratory condition bronchopulmonary dysplasia (BPD). These are major life threatening disorders that constitute an unmet medical need. BPD and Infant Respiratory Distress Syndrome combined are responsible for most premature (non-accidental) infant morbidity and mortality in developed countries. BPD alone ranks with cystic fibrosis and asthma as one of the most common lung diseases in infants with approximately 5,000 to 10,000 associated new cases (annually) of BPD in the US alone. Children with BPD are at an increased risk for acquiring pneumonia and bronchiolitis from a number of opportunistic lung pathogens, including respiratory syncytial virus. As such, the overall burden of cost for treating infants with BPD in the US is estimated to be US $2.4 billion, which is second only to asthma and exceeds cystic fibrosis. Current therapies for pediatric disorders (e.g. positive pressure ventilation, supplemental oxygen) are often unable to prevent, and indeed can contribute to, chronic morbid sequelae.

While BPD was originally described as a sequela of lung injury in large preterm infants (7), a “New BPD” has been recognized that occurs most frequently in newborns weighing less than 1 kg, who often display little or no lung disease at birth (6). The “New” BPD is characterized by abnormal development of the alveoli and the conducting airways, secondary to premature birth and the adverse effects of (life-saving) neonatal treatments such as supplemental oxygen and/or positive pressure ventilation. Growth and development during early childhood can compensate for these problems to a limited extent, especially in milder cases, suggesting that induction of the formation of new, functional alveolar space would represent an important clinical advance in these patients as well. Although multiple pathophysiologic mechanisms are likely to contribute to this “New BPD,” it is currently believed that it is largely due to an arrest in alveolarization.

Clarification of the molecular mechanisms that drive fetal lung organogenesis may contribute to the identification of specific molecular targets for therapeutic intervention to prevent or ameliorate the massive burden of neonatal and chronic lung disease. For this reason, there continues to be considerable interest in fetal lung development and its regulation.

Mammalian Lung Organogenesis

The embryonic human lung expands into a respiratory exchange organ with a surface area of 70 m². The development of this complex diffusible “breathing” interface is dependent on the spatiotemporally coordinated and exquisitely regulated developmental processes of branching morphogenesis, migration, differentiation, vasculogenesis, and alveolarization. The molecular determinants of all of these events include transcription factors, peptide growth factors, and their cognate receptors as well as cellular and extracellular signaling molecules. Alveolarization comprises the processes by which the gas exchange units of the lungs form, and by which such processes are regulated or may be modified. Branching morphogenesis comprises the processes by which the branched structures of an organ, such as the lungs and kidneys among others, are formed, and also by which these processes are regulated or may be modulated.

The histological development of the mouse lung has been divided into four stages: [1] pseudoglandular stage; development of the bronchial and respiratory tree, formation of the undifferentiated primordial system; [2] canalicular stage; development of terminal sacs and early vascularization; [3] terminal sac stage; increase in number of terminal sacs and vascularization, differentiation of type 1 and 2 alveolar cells (AEC 1, AEC 2); and [4] alveolar stage, when terminal sacs develop into mature alveolar ducts and alveoli (2). Alveolar development varies temporally between species; in rodents it is largely postnatal, whereas in humans it is already relatively advanced at birth.

In mammals, the two main bronchi and the respiratory tree of the lung are generated by sequential rounds of branching morphogenesis of two primary buds that arise on the ventrolateral wall of the embryonic foregut (21) on mouse Embryonic Day 9 (human Day 25). The primary bronchi grow and sprout secondary bronchi, which in turn sprout tertiary bronchi. In the developing lung, buds emerge in a characteristic temporal and spatial order. Successive branches diminish in size and the epithelial structure reorganizes.

As the airways develop, multipotential epithelial cells differentiate and localize in a distinct proximo-distal pattern. Upper airways are lined with ciliated columnar cells and mucus-secreting cells, lower airways are lined with Clara cells, and alveoli are lined with alveolar type 1 and 2 epithelial cells (AEC 1 and 2). The pulmonary interstitium comprises several specialized mesenchymal lineages including fibroblasts, myofibroblasts, and smooth muscle cells. The multipotential epithelium of early embryonic lung can be induced to express specific peripheral cell lineages by soluble mesenchyme-derived factors. In parallel with the inductive effects of mesenchymal signaling on epithelial branching morphogenesis, retrograde signals from the branching epithelium contribute to patterning, growth, and differentiation of the surrounding mesenchyme.

An alveolus is the functional unit of bi-directional gas exchange in the lung. In humans, morphogenesis of alveoli occurs mainly postnatally with an increase of 280 million alveoli during the first eight years of human life. Alveoli arise initially from outgrowths of terminal air ducts, a process coincident with the appearance of AEC 2 cells. Type 2 cells subsequently proliferate, co-assemble and serve both as progenitors for attenuated type 1 alveolar cells and later as the only source of pulmonary surfactant. A mature alveolus consists of a central air space lined by type 2 and type 1 alveolar cells which are adherent to a thin basal basement {McGowan, 1992 FASEB J. 6:2895-2904; U.S. Pat. No. 5,696,229}. Some residual potential for transdifferentiation between cell types (i.e., transitions from AEC 2 to AEC 1 phenotype) exists in the alveolar epithelium. Following lung injury, AECs regain the capacity to proliferate and repopulate the damaged alveolar epithelium. Repair after injury to the adult lung also engages some of the same factors that regulate lung development.

Mesenchymal-Epithelial and Cell-Matrix Interactions Regulate Lung Branching and Cytodifferentiation

Morphogenesis and differentiation of the fetal lung depend upon precise signaling between mesenchymal and epithelial cells, and between cellular and extracellular matrix (ECM) components (25-28). These signaling events regulate cell proliferation, fate, migration, and differentiation (29). Lung buds in explant culture develop into extensively branched structures (30), suggesting that essential patterning information is contained in the epithelium and surrounding mesenchyme of the isolated tissue. Transplantation of peripheral lung mesenchyme onto Day 11 embryonic mouse tracheal explants in culture results in induction of both pulmonary branching and expression of genes specific to peripheral epithelial cell lineages (30-32). Indeed, classical tissue recombination experiments demonstrated that induction of early embryonic lung branching morphogenesis could be achieved in the absence of mesenchymal tissue when soluble mesenchymal factors were present (33,34). Induction of tracheal branching occurred during a restricted window of lung development.

During the period of active branching, lung bud outgrowth is marked by localized reduction in extracellular matrix (ECM) components accompanied by a corresponding increase in epithelial proliferation (37). In the late canalicular lung, remodeling of the ECM facilitates direct communication between maturing epithelial and mesenchymal cells. At this time, the distal epithelium flattens and terminal differentiation begins, marked by the onset of rapid surfactant production (26).

Late Gestation Lung 1 Gene

Major advances in our understanding of the molecular embryology of the fetal lung have occurred in the past decade. Nevertheless, the molecular determinants that modulate the intricate processes of fetal lung branching and differentiation remain incompletely understood. In order to investigate the molecular mechanisms by which fibroblasts mediate glucocorticoid action on epithelial cells in late gestation lung, glucocorticoid inducible genes were identified in developing lung in a fetal rat model (Kaplan F. et al., (1999) Am J. Physiol. 276 (Lung Cell. Mol. Physiol. 20) L1027-L1036). The techniques of differential display-PCR, library screening and a novel approach for the specific amplification of cDNA ends (similar to that of Li et al) (Li and Nicholas, Biotechniques (1997) 22:1038-1044) were used to identify genes expressed in late gestation lung. 25% of expressed genes in fetal rat lung cell cultures were screened and a panel of cDNAs probes was isolated representing mRNAs whose pattern of developmental and hormonally modulated expression is coordinate with the onset of surfactant synthesis. A glucocorticoid-induced late gestation lung mesenchymal cDNA library was then probed with randomly selected sequence probes, and A gene isolated, late gestation lung 1 gene (LGL1). LGL1 is differentially expressed in fetal lung mesenchymal cells during development, but is not detectable in lung epithelium {Kaplan F. et al supra}.

A partial sequence of 3052 bp has been reported for LGL1, with a deduced polypeptide length of 188 amino acids (lgl1) (GenBank Accession No. AF109674; Kaplan F. et al. supra). LGL1 is glucocorticoid-induced and differentially expressed in fetal lung mesenchymal cells (fibroblasts) during development, but is not detectable in lung epithelium. Expression was also observed in fetal heart, kidney, intestine, liver and brain, and in adult lung and heart, kidney, intestine, liver and spleen. The coding region of this partial LGL1 sequence demonstrated 68% identity (81% homology) at the amino acid level to P25TI, a polypeptide with weak trypsin inhibitor activity previously found in human neuroblastoma and glioblastoma cells but not present in normal human tissues (GenBank Accession No. NP_{—}056970 {Yamakawa, T. et al, Biochim. Biophys. Acta 1395 (2), 202-208 (1998)}. Interestingly, virtually no significant homology was observed at the level of DNA sequence, suggesting lgl1 and P25TI are unlikely to have evolved from a single ancestral gene. (Kaplan F. et al., Am J. Physiol. (1999) 276 (Lung Cell. Mol. Physiol. 20):L1027-L1036).

Kaplan et al. proposed a model for a role of lgl1 in the regulation of ECM remodeling based on 1) the temporal and spatial pattern of lgl1 expression in fetal lung mesenchyme, 2) the enrichment of lgl1 in cysteine residues, 3) the likelihood that it is an extracellular protein (based on homology with the CRISP family of secreted proteins, maintaining two signature CRISP protein motifs in the deduced polypeptide), and 4) the homology of lgl1 to the trypsin inhibitor P25TI. LGL1 is maximally expressed by fibroblasts adjacent to the epithelium during the late canalicular early-saccular stage of lung development, a time and place of active proteolytic thinning of ECM. They postulated that lgl1 is secreted and acts to regulate ECM degradation by modulation of levels of trypsin activity. Trypsin, in turn, may be involved in ECM degradation either directly via hydrolysis of ECM proteins or indirectly by activation of matrix metalloproteinases.

Trexler et al. (Eur. J. Biochem. (2000) 267:5751-5757) extended the 3′ end of the initial (188 amino acid) sequence of rat fetal lung LGL1 (partial coding sequence GenBank Accession No. AY033439) to yield a polypeptide of 437 amino acids. They showed that the 437 amino acid polypeptide has homology with other proteins over a region they named the LCCL domain. NMR studies of the LCCL domain structure in Cochlear protein Coch-5b2 indicates that residues therein are important for protein folding and binding partner interactions (Liepinsh et al., Embo J. (2001) 20: 5347-53). They also speculated that LGL1 could play a role in antibody-independent defense against pathogens, as it is produced in the fetal lung at the same time as lung surfactant components which are thought to serve critical roles in this host defence system.

This background information is provided for the purpose of making known information believed by the applicant to be of possible relevance to the present invention. No admission is necessarily intended, nor should be construed, that any of the preceding information constitutes prior art against the present invention. Publications referred to throughout the specification are hereby incorporated by reference in their entireties in this application.

### SUMMARY OF THE INVENTION

An object of the present invention is to provide late gestation lung genes, fragments and uses thereof. In accordance with an aspect of the present invention there is provided, a nucleic acid molecule comprising a polynucleotide selected from the group consisting of:
(a) a polynucleotide with a sequence as set forth in SEQ ID NO: 1 or 3;
(b) a polynucleotide that encodes a protein with a sequence as set forth in SEQ ID NO: 2 or 4;
(c) a polynucleotide that is at least about 90% identical to the polynucleotide of (a) or (b);
(d) a polynucleotide fragment of the polynucleotide of (a), (b) or (c) which encodes a polypeptide fragment having at least one activity of lgl1;
(e) a polynucleotide capable of hybridizing under stringent conditions to the polynucleotide of (a), (b), (c) or (d).
(f) a polynucleotide capable of hybridizing under stringent conditions to a polynucleotide that is complementary to the polynucleotide of (a), (b), (c) or (d).

The invention also encompasses isolated nucleic acid homologues of the nucleic acid molecules of the invention and methods of obtaining or producing such homologues, as well as antisense oligonucleotide molecules complementary to the nucleic acid molecules of the invention. The nucleic acid molecules of the invention may further be operably linked to one or more expression control elements, including vectors comprising the isolated nucleic acid molecules. The invention further provides for host cells transformed with the nucleic acid molecules of the invention and methods to express polypeptides of the invention.

In accordance with another aspect of the invention, there is provided a polypeptide selected from the group consisting of:
(a) a polypeptide comprising the amino acid sequence of SEQ ID NO: 2 or 4;
(b) a polypeptide comprising a fragment of at least 6 contiguous amino acids of SEQ ID NO: 2 or 4;
(c) a polypeptide as in (a) or (b) comprising one or more conservative amino acid insertions, deletions or substitutions;
(d) a polypeptide comprising naturally occurring amino acid sequence variants of SEQ ID NO: 2 or 4; and
(e) a polypeptide comprising a fragment of lgl1.

The invention further provides for antibodies (monoclonal or polyclonal) or antigen binding fragments that specifically bind to the polypeptides of the invention.

The present invention also provides for compounds that specifically inhibit expression of the nucleic acid molecule of the invention, and antisense molecules capable of blocking expression of a polypeptide of the invention.

In accordance with another aspect of the invention, there is provided a nucleic acid molecule that modulates the expression of lgl1, wherein said nucleic acid molecule comprises a nucleotide sequence from the 5′ untranslated region of SEQ ID NO: 1 or 3, or a degenerate variant thereof.

In accordance with another aspect of the invention, there is provided a method of identifying an agent that modulates the expression of a nucleic acid molecule encoding the polypeptide of SEQ ID NO: 2 or 4 comprising:
(a) exposing cells which express the nucleic acid to the agent; and
(b) determining whether the agent modulates expression of said nucleic acid, thereby identifying an agent which modulates the expression of a nucleic acid encoding the polypeptide of SEQ ID NO: 2 or 4.

In accordance with another aspect of the invention, there is provided a method of modulating the expression of a nucleic acid molecule encoding the polypeptide of SEQ ID NO: 2 or 4 comprising using an effective amount of an agent which modulates the expression of a nucleic acid molecule encoding the polypeptide of SEQ ID NO: 2 or 4.

In accordance with another aspect of the invention, there is provided a method of identifying an agent that modulates at least one activity of the polypeptide of SEQ ID NO: 2 or 4 comprising:
(a) exposing cells which express the polypeptide to the agent; and
(b) determining whether the agent modulates at least one activity of said polypeptide, relative to control cells not exposed to said agent.

In accordance with another aspect of the invention, there is provided a method of modulating at least one activity of the polypeptide of SEQ ID NO: 2 or 4 comprising using an effective amount of an agent which modulates said activity of the polypeptide of SEQ ID NO: 2 or 4.

In accordance with another aspect of the invention, there is provided a method of identifying an agent that modulates one activity of the polypeptide of SEQ ID NO: 2 or 4 comprising:
(a) exposing cells which express the polypeptide to the agent; and
(b) determining whether the agent modulates one activity of said polypeptide, relative to control cells not exposed to said agent

In accordance with another aspect of the invention, there is provided a method of modulating one activity of the polypeptide of SEQ ID NO: 2 or 4 in cells comprising administering an effective amount of an agent which modulates one activity of the polypeptide of SEQ ID NO: 2 or 4 to said cells.

In accordance with another aspect of the invention, there is provided a method of identifying binding partners for the polypeptide of SEQ ID NO: 2 or 4 comprising:
(a) exposing said polypeptide to at potential binding partner; and
(b) determining whether the potential binding partner binds to said protein, thereby identifying binding partners for the polypeptide of SEQ ID NO: 2 or 4.

The invention additionally provides for methods of diagnosis comprising the step of determining the presence, absence or level of expression of a nucleic acid molecule of the invention, or of a polypeptide of the invention, in an animal using an antibody.

In accordance with another aspect of the invention, there is provided a method of modulating lung disease in an animal by administering an effective amount of a nucleic acid expressing lgl1 or a fragment thereof to said animal.

In accordance with another aspect of the invention, there is provided a method of modulating airway branching in an animal comprising administering an effective amount of a nucleic acid expressing lgl1 or a fragment thereof to said animal.

In accordance with another aspect of the invention, there is provided a method of modulating abnormal alveolarization in an animal by administering an effective amount of a nucleic acid expressing lgl1 or a fragment thereof to said animal.

In accordance with another aspect of the invention, there is provided a method of modulating airway branching, abnormal alveolarization or lung disease in an animal comprising the steps of:
(a) transforming cells in vitro with a nucleic acid encoding lgl1 or a fragment thereof;
(b) selecting the cells transformed in step (a) that express lgl1 or a fragment thereof; and
(c) introducing the cells selected in step (b) into the animal, thereby modulating airway branching, abnormal alveolarization or lung disease in an animal.

In accordance with another aspect of the invention, there is provided a method of modulating lung disease in an animal by administering an effective amount of lgl1 or a fragment thereof to said animal.

In accordance with another aspect of the invention, there is A method of modulating airway branching in an animal comprising administering an effective amount of lgl1 or a fragment thereof to said animal.

In accordance with another aspect of the invention, there is provided a method of modulating abnormal alveolarization in an animal by administering an effective amount of lgl1 or a fragment thereof to said animal.

The invention further provides for pharmaceutical compositions and uses of the nucleic acid molecules and polypeptides to prepare medicaments for use in the treatment of disease. In one embodiment of the invention the disease is a lung disease or disorder, such as BPD, emphysema, New BPD, COPD, CDH, and chronic bronchial infection.

### BRIEF DESCRIPTION OF THE FIGURES

FIG. 1 shows the temporal expression of LGL1 mRNA in fetal rat lung. (a) Ethidium bromide-stained agarose gels showing LGL1 RT-PCR products (472 bp bands) from days 12-15 of gestation after 27 cycles of amplification. Control β-actin RT-PCR (515 bp; 25 cycles). (b) Northern blots for LGL1 and control GAPDH at days 16-21 gestation. LGL1 expression is evident from the earliest day tested (d12), and maximal at gestational day 21.

FIG. 2 illustrates LGL1 mRNA localization by non-radioactive in situ hybridization. Mesenchyme-specific labelling is present throughout gestational days 13-21 (purple staining). Mesenchymal cells were identified by immunochemistry for vimentin (brown) on serial tissue sections. During the pseudoglandular stage of lung development (A), LGL1 mRNA is spread in a uniform pattern throughout the mesenchyme surrounding the lung bud. Later in gestation (D,H), LGL1 expression becomes “patchy” and is localized to cells positive for (smooth muscle) α-actin (brown, F,J).

FIG. 3 shows the temporal expression of lgl1 protein in embryonic rat lung. Western blot analysis on whole lung tissue extracts in the fetal rat lung (upper panel) and lgl1 peptide competition (bottom panel). At all gestational ages tested (d18-d21), lgl1 antibody identifies a band at approximately 52 kDa, which is the predicted molecular weight of lgl1 protein. Detection of lgl1 protein expression is substantially removed by adding a 5 fold molar excess of the peptide (SEQ ID NO: 14) used in the generation of the antibody.

FIG. 4 illustrates immunostaining for lgl1 protein in fetal rat lung sections, using a polyclonal rabbit anti rat lgl1 antibody. In (A) d16, (B) d19, (C) d20, (D) d22 fetal rat lung sections, lgl1 protein is localized to the mesenchyme in a patchy fashion. Immunochemistry for vimentin (brown) labelled all mesenchymal cells of the lung (B, D, F, H). Minimal background was observed in tissue sections incubated with non-immune rabbit IgG (not shown).

FIG. 5 demonstrates that oligodeoxynucleotides (ODNs) antisense (As) to LGL1 specifically inhibit branching morphogenesis in fetal rat lung explant cultures. (A) Gestational day 13 lung; (B) day 13 lung, showing normal branching morphogenesis after culture for 48 hours in serum-free medium. Lungs were maintained in explant culture for 48 hours in 3% O₂/N₂ in the presence of 30 μM LGL1 ODNs: (C) Sense sequence, (D) random “SCRambled” sequence or (E) antisense sequence.

FIG. 6 demonstrates that LGL1 antisense oligodeoxynucleotides (ODNs) decrease the number of terminal airway buds. Lung rudiments were maintained in serum free medium for 48 hours in the presence of either 30 μM or 40 μM ODNs, antisense (As), sense (S) or scrambled (SCR). Results are the sum of 4 lungs/condition, in each of 3 independent experiments. *p<0.02, ** p<0.001, compared with controls.

FIG. 7 shows quantitative data on branching over time. Day 12 lung rudiments were cultured for 48 hours in the presence of 40 μM of antisense, control sense, control scrambled oligodeoxynucleotides or medium only. Terminal airway buds were counted every 24 hours. Results are the sum of 4 lungs/condition, in each of 3 independent experiments. *p<0.05, compared with controls.

FIG. 8 shows the effect of antisense LGL1 oligodeoxynucleotides (ODNs) on branching morphogenesis over time. Day 12 lungs were maintained for 48 hours in the presence of 40 μM LGL1 ODNs: antisense (C, F), sense (B, E) or medium only (A, D). Progressive branching over time is evident in sense, scrambled (not shown) and medium controls, but not in antisense treated lungs. The difference is more pronounced after 48 hours than after 24 hours.

FIG. 9 illustrates the inhibition of LGL1 mRNA expression (A) and lgl1 protein (B) in fetal rat lung by antisense oligodeoxynucleotides (ODNs). Day 13 lung rudiments were cultured for 48 hours in the absence (M, medium alone) or presence of 40 μM ODNs: antisense (As), sense (S) or scrambled (SCR). Message for LGL1 was assessed by low cycle RT-PCR followed by southern blot analysis for LGL1 and the control β-actin. Lgl1 protein expression was detected using a polyclonal anti lgl1 antibody. (C) Bands were quantified by densitometric analysis, normalized to control cultures exposed to medium only. Lgl1 protein levels in As-treated explants are reduced by 51% relative to controls (mean±SEM; p<0.05; n=3 independent experiments, 4 lungs/condition).

FIG. 10 illustrates lgl1 protein expression in lung tissue in a neonatal rat model of bronchopulmonary dysplasia (BPD). A rat model of BPD was generated by exposing neonatal rats to 60% oxygen for 7 days and control litters received air for the same period. Following the experiment lung tissue was fixed and sectioned. Expression of lgl1 protein was detected using immunohistochemistry and a polyclonal anti-lgl1 antibody. Reduced amounts of lgl1 protein is observed in BPD rats (C) compared to 7 day control rats (D). (A) and (B) represent negative control staining (no lgl1 staining) for BPD and control neonatal rats, respectively.

FIG. 11 illustrates lgl1 protein expression in lung tissue in a neonatal rat model of bronchopulmonary dysplasia (BPD). A rat model of BPD was generated by exposing neonatal rats to 60% oxygen for 14 days and control litters received air for the same period. Following the experiment lung tissue was fixed and sectioned. Expression of lgl1 protein was detected using immnunohistochemistry and a polyclonal anti-lgl1 antibody. Reduced amounts of lgl1 protein is observed in BPD rats (C) compared to 14 day control rats (D). Similarly, there is diminished lgl1 staining at day 14 than at day 7 in BPD neonatal rats. (A) and (B) represent negative control staining (no lglg1 staining) for BPD and control neonatal rats, respectively.

FIG. 12 illustrates a Western blot analysis with anti-flag m2 antibodies verifying the production of the lgl1-flag protein in transfected HEK 293T cells. The hVeriscan-LGL1 transgene construct was inserted into HEK293T cells. Western blot analysis of HEK 293T cells samples at 24 and 48 h post-transfection was determined using anti-flag m2 antibodies and confirmed the generation of the lgl1-flag protein (arrow). Mock transfected HEK 293T cells were used as a negative control and did not produce lgl1-flag protein.

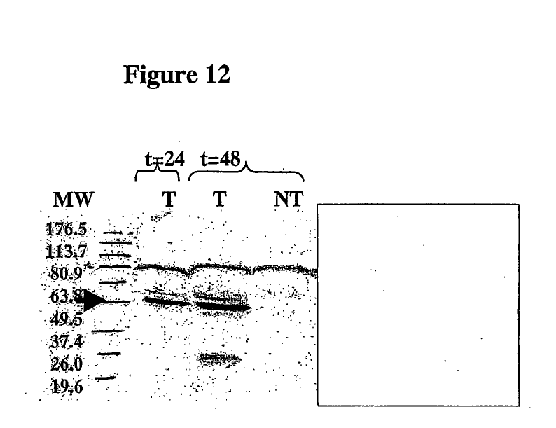

FIG. 13 depicts the hVersican-LGL1 Transgene construct. The hVersican-LGL1 transgene was constructed by ligation of a 1.51 kb fragment of rat LGL1 cDNA with C-terminal FLAG tag epitope into the Not I site of a vector containing the 839 bp-hVersican promoter region with a synthetic slice donor/acceptor site, and a poly-adenylation (pA) signal sequence at the 3′ end.

FIG. 14 shows gel electrophoresis of restriction enzyme digest products confirming correct transgene orientation. The proper orientation of the hVersican-LGL1 transgene was assessed by digestion of the construct and agarose gel electrophoresis of the digestion products. Enzymatic digestion generated fragments of the expected sizes confirming the correct orientation of the transgene (3.35 kb and 2.1 kb).

FIG. 15 illustrates gel electrophoresis of PCR products obtained with primers designed for use in identifying hVersican-LGL1-FLAG transgenic mice. Two sets of primers were used to generate a 620 bp (set 1) and a 553 bp (set 2) PCR product, shown on an agarose gel electrophoresis. The 5′ primer anneals to the hVeriscan promoter sequence and the 3′ primer to rat LGL1 coding sequences. Duplicate samples were run of the product generated using each primer set.

FIG. 16 illustrates the LGL1 transgenic genotype in hVersican-LGL1 transgenic mice. PCR genotyping revealed that the hVersican-LGL1 transgene can be expressed in LGL1 transgenic mice. Lane 1 and 2 represent hVersican-LGL1 transgenic mice that do not express the hVersican-LGL1 transgene and lane 3 illustrates a hVersican-LGL1 transgenic mouse that over-expresses this transgene. Lane 4 is a positive control using the hVersican-LGL1 plasmid DNA and lane 5 represents a negative control using water as template.

### DETAILED DESCRIPTION OF THE INVENTION

### I. GENERAL DESCRIPTION

The present invention pertains to a gene family that is differentially expressed during the late gestational stage of fetal lung development in mesenchymal cells compared to epithelial cells. This gene family includes genes encoding for lgl polypeptides of the rat and human, as well as genes encoding for polypeptides with substantially similar activities to lgl1 in other tissues or other animal species, particularly mammalian species.

The proteins of the invention or genes encoding same can serve as a target for agents that modulate gene expression or peptide activity. For example, agents may be identified that modulate biological processes associated with organ development, including but not limited to biological processes in kidney, brain or pancreas development or lung development such as branching morphogenesis or alveolarization, further including biological processes associated with disorders of tissues such as the lungs or kidneys, brain or pancreas.

The proteins and nucleic acids of the invention may also be used or targeted in the development of therapeutic interventions to prevent,.ameliorate or treat disorders of developing and mature tissues, such as the lungs, heart, liver, kidney, brain, pancreas and spleen.

The present invention is further based on the development of methods for isolating binding partners that bind to the protein. Additionally, the protein provides targets for the screening of synthetic small molecules and combinatorial or naturally occurring compound libraries to discover therapeutics to regulate lung development and function.

Definitions

Unless defined otherwise, all technical and scientific terms used herein have the same meaning as commonly understood by one of ordinary skill in the art to which this invention belongs. Generally, the nomenclature used herein and the laboratory procedures in spectroscopy, drug discovery, cell culture, molecular genetics, plastic manufacture, polymer chemistry, diagnostics, amino acid and nucleic acid chemistry, and sugar chemistry described below are those well known and commonly employed in the art. Standard techniques are typically used for preparation of plastics, signal detection, recombinant nucleic acid methods, polynucleotide synthesis, and microbial culture and transformation (e.g., electroporation, lipofection).

The techniques and procedures are generally performed according to conventional methods in the art and various general references (see generally, Sambrook et al. Molecular Cloning: A Laboratory Manual, 2d ed. (1989) Cold Spring Harbor Laboratory Press, Cold Spring Harbor, N.Y., and Lakowicz, J. R. Principles of Fluorescence Spectroscopy, New York: Plenum Press (1983) for fluorescence techniques, which are incorporated herein by reference) which are provided throughout this document. Standard techniques are used for chemical syntheses, chemical analyses, and biological assays.

### II. SPECIFIC EMBODIMENTS

A. The Proteins of the Invention

The present invention provides a family of proteins related to lgl1 proteins, including isolated lgl1 proteins, allelic variants of the lgl1 proteins, insertion, deletion or conservative amino acid substitution or other variants of the lgl1 proteins, and derivatives, analogue, and biologically active fragments thereof.

As used herein, lgl1 “protein”, “peptide” or “polypeptide” refers, in part, to a protein that has the rat amino acid sequence depicted in SEQ ID NO: 2 or the human amino acid sequence depicted in SEQ ID NO: 4. The proteins of the present invention include naturally occurring allelic variants of SEQ ID NO: 2 or 4, and the corresponding proteins of other animal species including, but not limited to, rabbit, mouse, rat, porcine, bovine, ovine, equine, human and non-human primate species, and the alleles or other naturally occurring variants of the family of proteins. The methods used to identify and isolate other members of the family of proteins related to these proteins are described below. As used herein, “naturally occurring allelic variant” refers to a polypeptide sequence that is present in an organism (including viruses) that can be isolated from a source in nature and that has not been intentionally modified by man in the laboratory.

The proteins of the present invention further include insertion, deletion or conservative amino acid substitution variants of SEQ ID NO: 2 or 4. Such variants can contain one or more than one insertion, deletion or substitution variation, or a combination thereof, and such combinations can also include other types of variations as described herein. As is known in the art, an amino acid insertion or deletion can be located at the N-terminal, C-terminal or internally within the polypeptide sequence, for example fusion proteins. As used herein, variations refers to amino acid sequence alterations that do not adversely affect the biological functions of the protein. A substitution, insertion or deletion is said to adversely affect the protein when the altered sequence prevents or disrupts a biological function associated with the protein. For example, the overall charge, structure or hydrophobic/hydrophilic properties of the protein can be altered without adversely affecting a biological activity. Accordingly, the amino acid sequence can be altered in a variant polypeptide of the invention, for example to render the peptide more hydrophobic or hydrophilic, without adversely affecting the biological activities of the protein.

Contemplated variants further include those containing predetermined mutations created by, for example, homologous recombination, site-directed or PCR mutagenesis, and also include derivatives wherein the protein has been covalently modified by substitution, chemical, enzymatic, or other appropriate means with a moiety other than a naturally occurring amino acid. Examples of such moieties include, but are not limited to a detectable moiety such as an enzyme or radioisotope, or a fusion protein affinity tag such as GST, lacZ, His, or Fc moieties. Modification of the structure of the polypeptides of the invention can be for such purposes as enhancing therapeutic or prophylactic efficacy, stability (e.g., ex vivo shelf life and resistance to proteolytic degradation in vivo), enabling detection in vitro or in vivo, or facilitating purification. Such modified peptides, when designed to retain at least one activity of the naturally occurring form of a protein of the invention are considered functional equivalents thereof.

Ordinarily, the members of the protein family and the derivatives and variants, including the allelic variants, the insertion, deletion and conservative substitution variants, will have an amino acid sequence having at least about 75%, 80%, 85%, 90% or 95% amino acid sequence identity with the sequence set forth in SEQ ID NO: 2 or 4. Identity or homology with respect to such sequences is defined herein as the percentage of amino acid residues in the candidate sequence that are identical with the known peptides, after aligning the sequences and introducing gaps, if necessary, to achieve the maximum percent homology, and not considering any conservative substitutions as part of the sequence identity (see section B for the relevant parameters). Fusion proteins, or N-terminal, C-terminal or internal extensions, deletions, or insertions into the peptide sequence shall not be construed as affecting homology. Protein sequence similarity is defined as the percentage of amino acid residues in the candidate sequence that are identical with the known peptides, after aligning the sequences and introducing gaps, if necessary, to achieve the maximum percent homology, and allowing for conservative substitutions as part of the sequence similarity (see section B for the relevant parameters). Conservative substitutions are well-known in the art (see also section G, below).

The proteins of the present invention may be in isolated form. As used herein, a protein is said to be isolated when physical, mechanical or chemical methods are employed to remove the protein from cellular constituents that are normally associated with the protein. A skilled artisan can readily employ standard purification methods to obtain an isolated protein.

The proteins of the present invention further include fragments of the proteins of the invention. As used herein, fragments have a consecutive sequence of at least about 3, 4, 5, 6, 7, 10, 15, 20, 25, 30, 35, 50, 75, 100, 150, 200, 300, 400 or more amino acid residues (a) of the sequence disclosed in. SEQ ID NO: 2 or 4; (b) of proteins that are members of the protein family; or (c) of derivatives or variants thereof, as defined above. A fragment is said to be “biologically active” when the fragment has at least one biological activity that is essentially, qualitatively the same activity as a protein of the invention. Such biologically active fragments, also referred to as peptides or polypeptides, may contain antigenic regions, functional regions of the protein identified as regions of the amino acid sequence which correspond to known protein domains, as well as regions of pronounced hydrophilicity. The regions are all easily identifiable by using commonly available protein sequence analysis programs known in the art. Other biological activities of the polypeptides of the invention, and biologically active fragments thereof, include but are not limited to modulation of alveolarization and morphogenesis, particularly branching morphogenesis, for example branching in the lung, kidney, brain, pancreas or tissues where LGL1 is expressed. Other activities include remodelling of the extracellular matrix and modulation of enzyme activity, for example inhibition of protease activity, in particular inhibition of trypsin activity. Optionally, the biologically active fragment of the invention also has other desirable properties. Fragments of the invention, including biologically active fragments of the invention, may be identified using methods of the invention, and used in methods of the invention.

Peptido-mimetic and organo-mimetic embodiments are also within the scope of the present invention, whereby the three-dimensional arrangement of the chemical constituents of such peptido-and organomimetics mimic the three-dimensional arrangement of the peptide backbone and component amino acid sidechains in the peptide, resulting in such peptido- and organomimetics of the peptides of this invention having substantial biological activity. It is implied that a pharmacophore exists for each of the described activities of the peptides of the invention. A pharmacophore is an idealised, three-dimensional definition of the structural requirements for biological activity. Peptido- and organomimetics can be designed to fit each pharmacophore with current computer modelling software (computer aided drug design). Overlap between the specific activities of pharmacophores may exist, but does not necessarily occur.

As described below, members of the family of proteins of the invention can be used: [1] to identify agents which modulate at least one activity of the protein; [2] to identify binding partners for the protein, [3] as an antigen to raise polyclonal or monoclonal antibodies; [4] as a therapeutic agent or target; and [5] as a diagnostic agent or marker.

B. Nucleic Acid Molecules

The nucleic acid molecules of the invention that encode the protein having SEQ ID NO: 2 or 4 or other proteins of the invention herein described, optionally in isolated form. As used herein, a “nucleic acid molecule” or “polynucleotide” of the invention is defined as RNA or DNA that encodes a protein of the invention as defined above, is complementary to a nucleic acid sequence encoding such peptides, hybridizes to the complement of the nucleic acid of SEQ ID NO: 1 or 3 and remains stably bound to it under appropriate stringency conditions, encodes a polypeptide sharing at least about 75%, 80%, 85%, 90% or 95% or more amino acid sequence identity with the sequence set forth in SEQ ID NO: 2 or 4. Specifically contemplated are genomic DNA, cDNA, mRNA and antisense molecules, as well as nucleic acids based on alternative backbones or including alternative bases whether derived from natural sources or synthesized. Such hybridizing or complementary nucleic acids, however, are defined further as being novel and unobvious over any prior art nucleic acid including that which encodes, hybridizes under appropriate stringency conditions, or is complementary to nucleic acid encoding a protein according to the present invention.

Homology or identity at the nucleotide or amino acid sequence level is determined by BLAST (Basic Local Alignment Search Tool) analysis using the algorithm employed by the programs blastp, blastn, blastx, tblastn and tblastx (Altschul, S. F. et al. (1997) Nucleic Acids Res 25:3389-3402 and Karlin et al., (1990) Proc. Natl. Acad. Sci. USA 87, 2264-2268, both fully incorporated by reference) which are tailored for sequence similarity searching. The approach used by the BLAST program is to first consider similar segments, with and without gaps, between a query sequence and a database sequence, then to evaluate the statistical significance of all matches that are identified and finally to summarize only those matches which satisfy a preselected threshold of significance. For a discussion of basic issues in similarity searching of sequence databases, see Altschul et al., (1994) (Nature Genetics 6, 119-129) which is fully incorporated by reference. The search parameters for histogram, descriptions, alignments, expect (i.e., the statistical significance threshold for reporting matches against database sequences), cutoff, matrix and filter (low complexity) are at the default settings. The default scoring matrix used by blastp, blastx, tblastn, and tblastx is the BLOSUM62 matrix (Henikoff et al., (1992) Proc. Natl. Acad. Sci. USA 89, 10915-10919, fully incorporated by reference) recommended for query sequences over 85 in length (nucleotide bases or amino acids).

For blastn, the scoring matrix is set by the ratios of M (i.e., the reward score for a pair of matching residues) to N (i.e., the penalty score for mismatching residues), wherein the default values for M and N are 5 and −4, respectively. Four blastn parameters were adjusted as follows: Q=10 (gap creation penalty); R=10 (gap extension penalty); wink=1 (generates word hits at every wink^{th} position along the query); and gapw=16 (sets the window width within which gapped alignments are generated). The equivalent Blastp parameter settings were Q=9; R=2; wink=1; and gapw=32. A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

“Stringent conditions” are those that (1) employ low ionic strength and high temperature for washing, for example, 0.015 M NaCl/0.0015 M sodium citrate/0.1% SDS at 50° C., or (2) employ during hybridization a denaturing agent such as formamide, for example, 50% (vol/vol) formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C. Another example is hybridization in 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at 42° C. in 0.2×SSC and 0.1% SDS. A skilled artisan can readily determine and vary the stringency conditions appropriately to obtain a clear and detectable hybridization signal. For example stringency may generally be reduced by increasing the salt content present during hybridization and washing, reducing the temperature, or a combination thereof. A more thorough treatise of such routine molecular biology techniques may be found in Sambrook et al., Molecular Cloning: A Laboratory Manual, Cold Spring Harbour Laboratory Press, Cold Spring Harbour, N.Y., (1989).

Nucleic acid molecules of the invention include those that hybridize under the above conditions to the complement of SEQ ID NO: 1 or 3 and that encode a functional protein. Other exemplary hybridizing molecules are those that hybridize under the above conditions to the complement strand of the open reading frame of SEQ ID NO: 1 or 3.

As used herein, a nucleic acid molecule is said to be “isolated” when the nucleic acid molecule is substantially separated from contaminant nucleic acid molecules encoding other polypeptides.

The nucleic acid molecules of the present invention further include fragments of the encoding nucleic acid molecules of the invention. As used herein, a fragment of an encoding nucleic acid molecule refers to a small portion of the entire protein coding sequence. The size of the fragment will be determined by the intended use. For example, if the fragment is chosen so as to encode an active portion of the protein, the fragment will need to be large enough to encode the functional region(s) of the protein. For instance, fragments which encode peptides corresponding to predicted antigenic regions may be prepared. If the fragment is to be used as a nucleic acid probe or PCR primer, then the fragment length is chosen so as to obtain a relatively small number of false positives during probing or priming (see the discussion in Section H).

Fragments of the encoding nucleic acid molecules of the present invention (i.e., synthetic oligonucleotides) that are used as probes or specific primers for the polymerase chain reaction (PCR), or to synthesize gene sequences encoding proteins of the invention, can easily be synthesized by chemical techniques, for example, the phosphotriester method of Matteucci et al., (1981) (J. Am. Chem. Soc. 103, 3185-3191) or using automated synthesis methods. In addition, larger DNA segments can readily be prepared by well known methods, such as synthesis of a group of oligonucleotides that define various modular segments of the gene, followed by ligation of oligonucleotides to build the complete modified gene.

The encoding nucleic acid molecules of the present invention may further be modified so as to contain a detectable label for diagnostic and probe purposes. A variety of such labels are known in the art and can readily be employed with the encoding molecules herein described. Suitable labels include, but are not limited to, biotin, radiolabeled nucleotides and the like. A skilled artisan can readily employ any such label to obtain labeled variants of the nucleic acid molecules of the invention.

Modifications to the primary structure itself by deletion, addition, or alteration of the amino acids incorporated into the protein sequence during translation can be made without destroying the activity of the protein. Such substitutions or other alterations result in proteins having an amino acid sequence encoded by a nucleic acid falling within the contemplated scope of the present invention.

As described below, members of the family of nucleic acid molecules of the invention can be used: (1) to identify agents which modulate at least one activity of the protein; (2) to identify binding partners for the protein, (3) to identify single nucleotide polymorphisms (SNPs), e.g. as markers of disease; (4) as a therapeutic agent or target; (5) as a probe; and (6) as a diagnostic agent or marker.

C. Isolation of Other Related Nucleic Acid Molecules

As described above, nucleic acid molecule having SEQ ID NO: 1 or 3, homologues thereof and other nucleic acid molecules of the invention, can be identified and characterized to encode other proteins of the invention in addition to the amino acid sequences herein described.

For instance, a skilled artisan can readily use the amino acid sequence of SEQ ID NO: 2 or 4 or of other proteins of the invention to generate antibody probes to screen expression libraries prepared from appropriate cells. Typically, polyclonal antiserum from mammals such as rabbits immunized with the purified protein (as described below) or monoclonal antibodies can be used to probe a mammalian cDNA or genomic expression library, such as lambda gtlI library, to obtain the appropriate coding sequence for other members of the protein family. The cloned cDNA sequence can be expressed as a fusion protein, expressed directly using its own control sequences, or expressed by constructions using control sequences appropriate to the particular host used for expression of the enzyme.

Alternatively, a portion of the coding sequence herein described can be synthesized and used as a probe to retrieve DNA encoding a member of the protein family from any mammalian organism. Oligomers containing approximately 18-20 nucleotides (encoding about a 6-7 amino acid stretch) are prepared and used to screen genomic DNA or cDNA libraries to obtain hybridization under stringent conditions or conditions of sufficient stringency to eliminate an undue level of false positives.

Additionally, pairs of oligonucleotide primers can be prepared for use in a polymerase chain reaction (PCR) to selectively clone an encoding nucleic acid molecule. A PCR denature-anneal-extend cycle for using such PCR primers is well known in the art and can readily be adapted for use in isolating other encoding nucleic acid molecules.

Nucleic acid molecules encoding other members of the protein family may also be identified in existing genomic or other sequence information using any available computational method, including but not limited to: PSI-BLAST (Altschul, et al. (1997) Nucleic Acids Res. 25:3389-3402); PHI-BLAST (Zhang, et al. (1998), Nucleic Acids Res. 26:3986-3990), 3D-PSSM (Kelly et al. (2000) J. Mol. Biol. 299(2): 499-520); and other computational analysis methods (Shi et al. (1999) Biochem. Biophys. Res. Commun. 262(1):132-8 and Matsunami et. al. (2000) Nature 404(6778):601-4.

D. rDNA Molecules Containing a Nucleic Acid Molecule

The present invention further provides recombinant DNA molecules (rDNAs) that contain a coding sequence. As used herein, an rDNA molecule is a DNA molecule that has been subjected to molecular manipulation. Methods for generating rDNA molecules are well known in the art, for example, see Sambrook et al., (1989) Molecular Cloning—A Laboratory Manual, Cold Spring Harbor Laboratory Press. In exemplary rDNA molecules, a coding DNA sequence is operably linked to expression control sequences and/or vector sequences in an expression vehicle. Suitable expression vehicles include plasmids, viral particles, and phage.

The choice of vector and/or expression control sequences to which one of the protein family encoding sequences of the present invention is operably linked depends directly, as is well known in the art, on the functional properties desired, e.g., protein expression, and the host cell to be transformed. For example, for protein expression in insect cells, baculovirus expression vectors are suitable. A vector contemplated by the present invention is at least capable of directing the replication or insertion into the host chromosome, and possibly also expression, of the structural gene included in the rDNA molecule.

Expression control elements that are used for regulating the expression of an operably linked protein encoding sequence are known in the art and include, but are not limited to, inducible promoters, constitutive promoters, secretion signals, and other regulatory elements. An exmeplary inducible promoter is readily controlled, such as being responsive to a nutrient in the host cell's medium.

Specific initiation signals may also be required for efficient translation of inserted nucleic acid sequences. These signals include the ATG initiation codon and adjacent sequences. In cases where an entire native gene sequence or cDNA, including its own initiation codon and adjacent sequences, is inserted into the appropriate expression vector, no additional translational control signals may be needed. In other cases, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators (Bittner et al. (1987) Methods in Enzymol. 153, 516).

In one embodiment, the vector containing a coding nucleic acid molecule will include a prokaryotic replicon, i.e., a DNA sequence having the ability to direct autonomous replication and maintenance of the recombinant DNA molecule extrachromosomally in a prokaryotic host cell, such as a bacterial host cell, transformed therewith. Such replicons are well known in the art. In addition, vectors that include a prokaryotic replicon may also include a gene whose expression confers a detectable marker such as a drug resistance. Typical bacterial drug resistance genes are those that confer resistance to ampicillin or tetracycline.

Vectors that include a prokaryotic replicon can further include a prokaryotic or bacteriophage promoter capable of directing the expression (transcription and translation) of the coding gene sequences in a bacterial host cell, such as E. coli. A promoter is an expression control element formed by a DNA sequence that permits binding of RNA polymerase and transcription to occur. Promoter sequences that are compatible with bacterial hosts are typically provided in plasmid vectors containing convenient restriction sites for insertion of a DNA segment of the present invention. Typical of such vector plasmids are pUC8, pUC9, pBR322 and pBR329 available from BioRad Laboratories, (Richmond, Calif.), pPL and pKK223 available from Pharmacia (Piscataway, N.J.).

Expression vectors compatible with eukaryotic cells, for example those compatible with vertebrate cells such as lung cells, can also be used to form rDNA molecules that contain a coding sequence. Eukaryotic cell expression vectors, including viral vectors, are well known in the art and are available from several commercial sources. Typically, such vectors are provided containing convenient restriction sites for insertion of the desired DNA segment. Typical of such vectors are pSVL and pKSV-10 (Pharmacia), pBPV-1/pML2d (International Biotechnologies, Inc.), pTDT1 (ATCC, #31255), the vector pCDM8 described herein, and the like eukaryotic expression vectors. Vectors may be modified to include lung cell specific promoters if needed.

Eukaryotic cell expression vectors used to construct the rDNA molecules of the present invention may further include a selectable marker that is effective in an eukaryotic cell, for example a drug resistance selection marker. An exemplary drug resistance marker is the gene whose expression results in neomycin resistance, i.e., the neomycin phosphotransferase (neo) gene. (Southern et al., (1982) J. Mol. Anal. Genet. 1, 327-341). Alternatively, the selectable marker can be present on a separate plasmid, and the two vedtors are introduced by co-transfection of the host cell, and selected by culturing in the appropriate drug for the selectable marker.

In mammalian host cells, a number of viral-based expression systems can be utilised. In cases where an adenovirus is used as an expression vector, the coding sequence of the nucleic acid of the invention can be ligated to an adenovirus transcriptionltranslation control complex, e.g., the late promoter and tripartite leader sequence. This chimeric gene can then be inserted into the adenovirus genome by in vitro or in vivo recombination. Insertion into a non-essential region of the viral genome (e.g., region E1 or E3) will result in a recombinant virus that is viable and capable of expressing a gene product in infected hosts (see, e.g., Logan (1984) Proc. Natl. Acad. Sci. USA 81, 3655).

Expression vectors compatible with plant cells can also be used to form rDNA molecules that contain a coding sequence of the present invention. Viral expression vectors (e.g., cauliflower mosaic virus and tobacco mosaic virus) and plasmid expression vectors (e.g., Ti plasmid) are suitable for use in plant cells. For example, Autographa californica nuclear polyhedrosis virus (AcNPV), which grows in Spodoptera frugiperda cells, is used as a vector to express foreign genes. A nucleic acid coding sequence of the invention can be cloned into non-essential regions of the virus (for example, into the polyhedrin gene region) and placed under control of an AcNPV promoter, e.g., the polyhedrin promoter. Successful insertion of a gene encoding a polypeptide or protein of the invention will result in inactivation of the polyhedrin gene and production of non-occluded recombinant virus (i.e., virus lacking the proteinaceous coat encoded by the polyhedrin gene). These recombinant viruses are then used to infect spodoptera frugiperda cells in which the inserted gene is expressed (see, e.g., Smith et al. (1983) J. Virol. 46, 584; Smith, U.S. Pat. No. 4,215,051).

E. Host Cells Containing an Exogenously Supplied Coding Nucleic Acid Molecule

The present invention further provides host cells transformed with a nucleic acid molecule that encodes a protein of the present invention. Those skilled in the field of molecular biology will understand that any of a wide variety of expression systems can be used to provide a recombinant protein. The precise host cell used is not critical to the invention. The host cell can be prokaryotic, eukaryotic or a plant cell.

In addition, a host cell may be chosen which modulates the expression of the inserted sequences, or modifies and processes the gene product in a specific, desired fashion. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product can be used. Such mammalian host cells include, but are not limited to, CHO, VERO, BHK, HeLa, COS, MDCK, 293, 3T3, WI38, and in particular, lung cell lines.

Cells useful for expression of a protein of the invention are not limited, so long as the cell line is compatible with cell culture methods and compatible with the propagation of the expression vector and expression of the gene product. Exemplary host cells include, but are not limited to, yeast, insect and mammalian cells, for example vertebrate cells such as those from a mouse, rat, monkey or human cell line. Exemplary eukaryotic host cells include cells of Saccharomyces or Pichia, Chinese hamster ovary. (CHO) cells available from the ATCC as CCL-61, NIH Swiss mouse embryo cells (NIH/3T3) available from the ATCC as CRL-1658, baby hamster kidney cells (BHK), COS cells available from the ATCC as CRL-1650, 293, or HeLa cells and like eukaryotic tissue culture cell lines. Further examples include primary tracheobronchial cells, primary airway epithelial cells, fetal rat lung epithelial cells including those adjacent and those peripheral to fibroblasts (N. B. Sweezey et al., (1998) Am. J. Physiol. (Lung Cell Mol. Physiol.) L103-L109) and adult Type 2 alveolar epithelial cells (AEC2) (Mason, R. J. et al., (1982) PNAS 79:6033-6037), A549, CALU 1, CALU 3, NHBE (Clonetics), HBE1, CFT1, MLE 15, CCD32LU (ATCC), L2 (rat fibroblast), IB3 (P L Zeitlin, Johns Hopkins Medical Institutes), H441, H661. Other useful cells include the cell lines of D. C. Gruenert, for example 16HBE14o(−), 9HTEo(−), CFBE45o(−), and CFBE 41o(−) (Winton H L et al., Clin Exp Allergy (1998) 28:1273-85; Hopfer U. et al., (1996) Am. J. Physiol. 270(1 Pt 1):C1-11; Kunzelmann K et al., Am. J. Respir. Cell Mol. Biol. (1993) 8:522-9; Gruenert D C et al., Am. J. Physiol. (1995) 268(3 Pt 1):L347-60).

Any prokaryotic host can be used to express a rDNA molecule encoding a protein of the invention. Exemplary prokaryotic hosts are E. coli and B. subtilis.

Transformation of appropriate cell hosts with a rDNA molecule of the present invention is accomplished by well known methods that typically depend on the type of vector used and host system employed. With regard to transformation of prokaryotic host cells, electroporation and salt treatment methods are typically employed, see, for example, Cohen et al., (1972) Proc. Natl. Acad. Sci. USA 69, 2110; and Sambrook et al., (1989) Molecular Cloning—A Laboratory Manual, Cold Spring Harbor Laboratory Press. With regard to transformation of vertebrate cells with vectors containing rDNAs, electroporation, cationic lipid or salt treatment methods are typically employed, see, for example, Graham et al., (1973) Virol. 52, 456; Wigler et al., (1979) Proc. Natl. Acad. Sci. USA 76, 1373-1376.

Successfully transformed cells, i.e., cells that contain a rDNA molecule of the present invention, can be identified by well known techniques including the selection for a selectable marker. For example, cells resulting from the introduction of an rDNA of the present invention can be cloned to produce single colonies. Cells from those colonies can be harvested, lysed and their DNA content examined for the presence of the rDNA using a method such as that described by Southern {J. Mol. Biol. (1975) 98:503} or Berent et al. {Biotech. (1985) 3:208} or the proteins produced from the cell assayed via an immunological method.

A number of selection systems can be used in stably-transfected mammalian cell lines, including but not limited to the herpes simplex virus thymidine kinase, hypoxanthine-guanine phosphoribosyltransferase, and adenine phosphoribosyltransferase genes can be employed in DHFR, tk, hgprt, or aprt cells, respectively. In addition, gpt, which confers resistance to mycophenolic acid (Mulligan et al. (1981) Proc. Natl. Acad. Sci. USA 78, 2072); neo, which confers resistance to the aminoglycoside G-418 (Colberre-Garapin et al. (1981) J. Mol. Biol. 150, 1); hygro, which confers resistance to hygromycin (Santerre et al. (1981) Gene 30, 147); and dihydrofolate reductase (DHFR) which confers resistence to methotrexate (as described in Ausubel et al., supra), can be used.

The host cells harbouring the expression vehicle can be cultured in conventional nutrient media adapted as need for activation of a chosen gene, repression of a chosen gene, selection of transformants, or amplification of a chosen gene.

For example, one expression system is the mouse 3T3 fibroblast host cell transfected with a pMAMneo expression vector (Clontech, Palo Alto, Calif.). pMAMneo provides an RSV-LTR enhancer linked to a dexamethasone-inducible MMTV-LTR promotor, an SV40 origin of replication which allows replication in mammalian systems, a selectable neomycin gene, and SV40 splicing and polyadenylation sites. DNA encoding a protein of the invention would be inserted into the pMAMneo vector in an orientation designed to allow expression. The recombinant protein could be isolated as described below. Other examples of host cells that can be used in conjunction with the pMAMneo expression vehicle include COS cells and CHO cells.

F. Production of Recombinant Proteins

The present invention further provides methods for producing a protein of the invention using nucleic acid molecules or the invention herein described. In general terms, a protein of the invention may be produced by any suitable method known in the art, including synthetic methods and recombinant methods.

The production of a recombinant form of a protein typically involves the following steps:

A nucleic acid molecule of the invention is first obtained that encodes a protein of the invention, such as a nucleic acid molecule comprising, consisting essentially of or consisting of SEQ ID NO: 1 or 3 or nucleotides 86-1576 (-1579 with the stop codon) of SEQ ID NO: 1, or nucleotides 32-1522 (-1525 with the stop codon) of SEQ ID NO: 3. If the encoding sequence is uninterrupted by introns, as is this open reading frame, it is directly suitable for expression in any host.

The nucleic acid molecule may then be placed in operable linkage with suitable control sequences, as described above, to form an expression unit containing the protein open reading frame. The expression unit is used to transform (transfect, transduce or infect) a suitable host and the transformed host is cultured under conditions that allow the production of the recombinant protein. Optionally the recombinant protein is isolated from the medium or from the cells; recovery and purification of the protein may not be necessary in some instances where some impurities may be tolerated.

Each of the foregoing steps can be done in a variety of ways. For example, the desired coding sequences may be obtained from genomic fragments and used directly in appropriate hosts. The construction of expression vectors that are operable in a variety of hosts is accomplished using appropriate replicons and control sequences, as set forth above. The control sequences, expression vectors, and transformation methods are dependent on the type of host cell used to express the gene and were discussed in detail earlier. Suitable restriction sites can, if not normally available, be added to the ends of the coding sequence so as to provide an excisable gene to insert into these vectors. A skilled artisan can readily adapt any host/expression system known in the art for use with the nucleic acid molecules of the invention to produce recombinant protein.

Alternatively

G. Methods to Chemically Modify a Polypeptide of the Invention to Improve its Biological Activity

Polypeptide variants of the invention can be produced, for instance, by amino acid substitution, deletion, or addition.

For example, it is reasonable to expect that an isolated replacement of a leucine with an isoleucine or valine, an aspartate with a glutamate, a threonine with a serine, or a similar replacement of an amino acid with a structurally related amino acid (i.e. isosteric and/or isoelectric mutations) will not have a major effect on the biological activity of the resulting molecule. Conservative replacements are those that take place within a family of amino acids that are related in their side chains. Genetically encoded amino acids are can be divided into four families:
(1) acidic=aspartate, glutamate;
(2) basic=lysine, arginine, histidine;
(3) nonpolar=alanine, valine, leucine, isoleucine, proline, phenylalanine, methionine, tryptophan; and
(4) uncharged polar=glycine, asparagine, glutamine, cysteine, serine, threonine, tyrosine.

In similar fashion, the amino acid repertoire can be grouped as
(1) acidic=aspartate, glutamate;
(2) basic=lysine, arginine, histidine;
(3) aliphatic=glycine, alanine, valine, leucine, isoleucine, serine, threonine, with serine and threonine optionally grouped separately as aliphatic-hydroxyl;
(4) aromatic=phenylalanine, tyrosine, tryptophan;
(5) amide=asparagine, glutamine; and
(6) sulphur-containing=cysteine and methionine. (See, for example, Biochemistry, 2nd ed., Ed. by L. Stryer, W H Freeman and Co.: 1981).

Whether a change in the amino acid sequence of a peptide results in a functional homologue (e.g. functional in the sense that the resulting polypeptide mimics or antagonises the wild-type form) can be readily determined by assessing the ability of the variant peptide to produce a response in cells in a fashion similar to the wild-type protein, or competitively inhibit such a response. Polypeptides in which more than one replacement has taken place can readily be tested in the same manner.

Generally, those skilled in the art will recognise that peptides of the invention as described herein may be modified by a variety of chemical techniques to produce compounds having essentially the same activity as the unmodified peptide, and optionally having other desirable properties. For example, carboxylic acid groups of the peptide, whether carboxyl-terminal or sidechain, may be provided in the form of a salt of a pharmaceutically-acceptable cation or esterified to form a C₁-C₁₆ ester, or converted to an amide of formula NR₁R₂ wherein R₁ and R₂ are each independently H or C₁-C₁₆ alkyl, or combined to form a heterocyclic ring, such as 5- or 6-membered. Amino groups of the peptide, whether amino-terminal or sidechain, may be in the form of a pharmaceutically-acceptable acid addition salt, such as the HCl, HBr, acetic, benzoic, toluene sulphonic, maleic, tartaric and other organic salts, or may be modified to C₁-C₁₆ alkyl or dialkyl amino or further converted to an amide. Hydroxyl groups of the peptide sidechain may be converted to C₁-C₁₆ alkoxy or to a C₁-C₁₆ ester using well-recognised techniques. Phenyl and phenolic rings of the peptide sidechain may be substituted with one or more halogen atoms, such as fluorine, chlorine, bromine or iodine, or with C1-C₁₆ alkyl, C₁-C₁₆ alkoxy, carboxylic acids and esters thereof, or amides of such carboxylic acids. Methylene groups of the peptide sidechains can be extended to homologous C₂ -C₄ alkylenes. Thiols can be protected with any one of a number of well-recognised protecting groups, such as acetamide groups.

Those skilled in the art will also recognise methods for introducing cyclic structures into the peptides of the invention to select and provide conformational constraints to the structure that result in enhanced binding and/or stability. For example, a carboxyl-terminal or amino-terminal cysteine residue can be added to the peptide, so that when oxidised the peptide will contain a disulphide bond, thereby generating a cyclic peptide. Other peptide cyclising methods include the formation of thioethers and carboxyl- and amino-terminal amides and esters.

In addition to peptides of the invention consisting only of naturally occurring amino acids, peptidomimetics or peptide analogues are also provided. Peptide analogues are commonly used in the pharmaceutical industry as non-peptide drugs with properties analogous to those of the template peptide. These types of non-peptide compound are termed “peptide mimetics” or “peptidomimetics” (Luthman, et al., A Textbook of Drug Design and Development, 14:386-406, 2nd Ed., Harwood Academic Publishers (1996); Grante (1994) Angew. Chem. Int. Ed. Engl. 33:1699-1720; Fauchere (1986) Adv. Drug Res. 15:29; Veber and Freidinger (1985) TINS, p.392; and Evans, et al. (1987) J. Med. Chem. 30:1229, which are incorporated herein by reference). Peptide mimetics that are structurally similar to therapeutically useful peptides may be used to produce an equivalent or enhanced therapeutic or prophylactic effect. Generally, peptidomimetics are structurally similar to a paradigm polypeptide (i.e., a polypeptide that has a biological or pharmacological activity), such as naturally-occurring receptor-binding polypeptide, but have one or more peptide linkages optionally replaced by a linkage selected from the group consisting of: —CH₂NH—, —CH₂S—, —CH₂—CH₂—, —CH═CH— (cis and trans), —COCH₂ —, —CH(OH)CH₂—, and —CH₂SO—, by methods known in the art and further described in the following references: Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley (1980) Trends Pharm. Sci. pp. 463-468, (general review); Hudson, et al. (1979) Int. J. Pept. Prot. Res., 14:177-185 (—CH₂NH—, CH₂CH₂—); Spatola, et al. (1986) Life Sci., 38:1243-1249 (—CH₂—S); Hann (1982) Chem. Soc. Perkin Trans. I, 307-314 (—CH═CH—, cis and trans); Almquist, et al. (1980) J. Med. Chem., 23:1392-1398, (—COCH₂—); Jennings-White, et al, (1982) Tetrahedron Lett. 23:2533, (—COCH₂—); Szelke, et al. (1982)European Appln. EP 45665 (—CH(OH)CH₂—); Holladay, et al. (1983) Tetrahedron Lett., 24:4401-4404 (—C(OH)CH₂—); and Hruby (1982) Life Sci., 31:189-199 (—CH₂—S—); each of which is incorporated herein by reference. A commom non-peptide linkage is —CH₂ NH—. Such peptide mimetics may have significant advantages over polypeptide embodiments, including, for example: more economical production, greater chemical stability, enhanced pharmacological properties (half-life, absorption, potency, efficacy, etc.), altered specificity (e.g., a broad-spectrum of biological activities), reduced antigenicity, and others.

Systematic substitution of one or more amino acids of a consensus sequence with a D-amino acid of the same type (e.g., D-lysine in place of L-lysine) may be used to generate more stable peptides. In addition, constrained peptides comprising a consensus sequence or a substantially identical consensus sequence variation may be generated by methods known in the art (Rizo, et al. (1992) Ann. Rev. Biochem., 61:387, incorporated herein by reference); for example, by adding internal cysteine residues capable of forming intramolecular disulphide bridges which cycles the peptide.

Synthetic or non-naturally occurring amino acids refer to amino acids which do not naturally occur in vivo but which, nevertheless, can be incorporated into the peptide structures described herein. Exemplary synthetic amino acids are the D-α-amino acids of naturally occurring L-α-amino acid as well as non-naturally occurring D- and L-α-amino acids represented by the formula H₂NCHR⁵COOH where R⁵ is 1) a lower alkyl group, 2) a cycloalkyl group of from 3 to 7 carbon atoms, 3) a heterocycle of from 3 to 7 carbon atoms and 1 to 2 heteroatoms selected from the group consisting of oxygen, sulphur, and nitrogen, 4) an aromatic residue of from 6 to 10 carbon atoms optionally having from 1 to 3 substituents on the aromatic nucleus selected from the group consisting of hydroxyl, lower alkoxy, amino, and carboxyl, 5)-alkylene-Y where alkylene is an alkylene group of from 1 to 7 carbon atoms and Y is selected from the group consisting of (a) hydroxy, (b) amino, (c) cycloalkyl and cycloalkenyl of from 3 to 7 carbon atoms, (d) aryl of from 6 to 10 carbon atoms optionally having from 1 to 3 substituents on the aromatic nucleus selected from the group consisting of hydroxyl, lower alkoxy, amino and carboxyl, (e) heterocyclic of from 3 to 7 carbon atoms and 1 to 2 heteroatoms selected from the group consisting of oxygen, sulphur, and nitrogen, (f) —C(O)R² where R² is selected from the group consisting of hydrogen, hydroxy, lower alkyl, lower alkoxy, and —NR³R⁴ where R³ and R⁴ are independently selected from the group consisting of hydrogen and lower alkyl, (g) —S(O)ₙR⁶ where n is an integer from 1 to 2 and R⁶ is lower alkyl and with the proviso that R⁵ does not define a side chain of a naturally occurring amino acid.

Other synthetic amino acids include amino acids wherein the amino group is separated from the carboxyl group by more than one carbon atom such as β-alanine, γ-aminobutyric acid, and the like.

Other embodiments of the proteins of the invention encompass covalent modifications to facilitate detection. “Detectable label” refers to materials, which when covalently attached to the peptides and peptide mimetics of this invention, permit detection of the peptide and peptide mimetics in vivo in the patient to whom the peptide or peptide mimetic has been administered. Suitable detectable labels are well known in the art and include, by way of example, radioisotopes, fluorescent labels (e.g., fluorescein), and the like. The particular detectable label employed is not critical and is selected relative to the amount of label to be employed as well as the toxicity of the label at the amount of label employed. Selection of the label relative to such factors is well within the skill of the art.

Covalent attachment of the detectable label to the peptide or peptide mimetic is accomplished by conventional methods well known in the art. For example, when the ¹²⁵I radioisotope is employed as the detectable label, covalent attachment of ¹²⁵I to the peptide or the peptide mimetic can be achieved by incorporating the amino acid tyrosine into the peptide or peptide mimetic and then iodinating the peptide (see, e.g., Weaner, et al., Synthesis and Applications of Isotopically Labelled Compounds, pp. 137-140 (1994)). If tyrosine is not present in the peptide or peptide mimetic, incorporation of tyrosine to the N or C terminus of the peptide or peptide mimetic can be achieved by well-known chemistry. Likewise, ³²P can be incorporated onto the peptide or peptide mimetic as a phosphate moiety through, for example, a hydroxyl group on the peptide or peptide mimetic using conventional chemistry.

H. Methods to Identify Binding Partners

The present invention provides for methods of isolating and identifying binding partners of proteins of the invention. In general, a protein of the invention is mixed with a potential binding partner or an extract or fraction of a cell under conditions that allow the association of potential binding partners with the protein of the invention. After mixing, peptides, polypeptides, proteins or other molecules that have become associated with a protein of the invention are separated from the mixture. The binding partner that bound to the protein of the invention can then be removed and further analysed. To identify and isolate a binding partner, the entire protein, for instance a polypeptide comprising the entire amino acid sequence of SEQ ID NO: 2 or 4 can be used. Alternatively, a fragment of the protein can be used, for example the fragment defined by amino acids 61-87 of the human LGL1 sequence (as set forth in SEQ ID NO: 13, fragments defining part or all of the SCP, secretary or LCCL domains, or fragments encompassing peptide domains that are otherwise defined, for example hydrophilic domains or epitopes.

As used herein, a cellular extract refers to a preparation or fraction that is made from a lysed or disrupted cell. As an example, cellular extracts will be cells derived from human lung or kidney tissue or cells, for instance, biopsy tissue or primary tissue culture cells. Alternatively, cellular extracts may be prepared from normal tissue or available cell lines, particularly lung or kidney derived cell lines.

A variety of methods can be used to obtain an extract of a cell. Cells can be disrupted using either physical or chemical disruption methods. Examples of physical disruption methods include, but are not limited to, sonication and mechanical shearing. Examples of chemical lysis methods include, but are not limited to, detergent lysis and enzyme lysis. A skilled artisan can readily adapt methods for preparing cellular extracts in order to obtain extracts for use in the present methods.

Once an extract of a cell is prepared, the extract is mixed with the polypeptide of the invention under conditions in which association of the polypeptide with the binding partner can occur. A variety of conditions can be used, for example conditions that closely resemble conditions found in the cytoplasm of a human cell may be used. Features such as osmolarity, pH, temperature, and the concentration of cellular extract used, can be varied to optimize the association of the protein with the binding partner.

After mixing under appropriate conditions, the bound complex is separated from the mixture. A variety of techniques can be used to separate the mixture. For example, antibodies specific to a protein of the invention can be used to immunoprecipitate the binding partner complex. Alternatively, standard chemical separation techniques such as chromatography-and density/sediment centrifugation can be used.

After removal of non-associated cellular constituents found in the extract, the binding partner can be dissociated from the complex using conventional methods. For example, dissociation can be accomplished by altering the salt concentration or pH of the mixture.

To aid in separating associated binding partner pairs from the mixed extract, the protein of the invention can be immobilized on a solid support. For example, the protein can be attached to a nitrocellulose matrix or acrylic beads. Attachment of the protein to a solid support aids in separating peptide/binding partner pairs from other constituents found in the extract. The identified binding partners can be either a single protein or a complex made up of two or more proteins. Alternatively, binding partners may be identified using a Far-Western assay according to the procedures of Takayama et al., (1997) Methods Mol. Biol. 69:171-184 or Sauder et al., (1996) J. Gen. Virol. 77:991-996 or identified through the use of epitope tagged proteins or fusion proteins, for example GST or His fusion proteins.

Alternatively, the nucleic acid molecules of the invention can be used in a yeast two-hybrid system to identify other binding partners. The yeast two-hybrid system is well known in the art and can readily be adapted to employ the nucleic acid molecules herein described.

I. Methods to Identify Agents that Modulate the Expression of a Nucleic Acid of the Invention

The invention provides methods for identifying agents that modulate the expression of a nucleic acid encoding a polypeptide of the invention such as a polypeptide having the amino acid sequence of SEQ ID NO:. 2 or 4. Such assays may use any available means of monitoring for changes in the expression level of the nucleic acids of the invention. As used herein, an agent is said to modulate the expression of a nucleic acid of the invention if it is capable of up- or down-regulating expression of the nucleic acid in a cell. One class of agents that modulates expression of the nucleic acid molecule of the invention is antisense molecules.

In one assay format, cell lines that contain reporter gene fusions between the open reading frame defined by nucleotides 86-1576 of SEQ ID NO: 1, or by nucleotides 32-1522 of SEQ ID NO: 3, and/or the 5′ and/or 3′ regulatory elements and any assayable fusion partner may be prepared. Numerous assayable fusion partners are known and readily available including the firefly luciferase gene and the gene encoding chloramphenicol acetyltransferase (Alam et al., (1990) Anal. Biochem. 188, 245-254). Cell lines containing the reporter gene fusions are then exposed to the agent to be tested under appropriate conditions and time. Differential expression of the reporter gene between samples exposed to the agent and control samples identifies agents which modulate the expression of a nucleic acid of the invention.

Additional assay formats may be used to monitor the ability of the agent to modulate the expression of a nucleic acid encoding a polypeptide of the invention, such as the protein having the sequence of SEQ ID NO: 2 or 4. For instance, mRNA expression may be monitored directly by hybridization to the nucleic acids of the invention. Cell lines are exposed to the agent to be tested under appropriate conditions and time and total RNA or mRNA is isolated by standard procedures such those disclosed in Sambrook et al., (1989) Molecular Cloning—A Laboratory Manual, Cold Spring Harbor Laboratory Press).

For example, cells may be derived from human lung or kidney tissue, e.g. lung tissue from biopsy, surgical resection or autopsy, or cultured lung cells from normal subjects or patients with a lung disorder. Exemplary cell types include primary tracheobronchial cells, primary airway epithelial cells, fetal rat lung epithelial cells including those adjacent and those peripheral to fibroblasts (N. B. Sweezey et al., (1998) Am. J. Physiol. (Lung Cell Mol. Physiol.) L103-L109) and adult Type 2 alveolar epithelial cells (AEC2) (Mason, R. J. et al., (1982) PNAS 79:6033-6037), A549, CALU 1, CALU 3, NHBE (Clonetics), HBE1, CFT1, MLE 15, CCD32LU (ATCC), L2 (rat fibroblast), IB3 (P L Zeitlin, Johns Hopkins Medical Institutes), H441, H661. Other useful cells include the cell lines of D. C. Gruenert, for example 16HBE14o(−), 9HTEo(−), CFBE45o(−), and CFBE 41o(−) (Winton H L et al., Clin Exp Allergy (1998) 28:1273-85; Hopfer U. et al., (1996) Am. J. Physiol. 270(1 Pt 1):C1-11; Kunzelmann K et al., Am. J. Respir. Cell Mol. Biol. (1993) 8:522-9; Gruenert D C et al., Am. J. Physiol. (1995) 268(3 Pt 1) :L347-60). Alternatively, cells from animal models of disease or other available cells or cell lines may be used.

Probes to detect differences in RNA expression levels between cells exposed to the agent and control cells may be prepared from the nucleic acids of the invention. It is an option, but not necessary, to design probes that hybridize only with target nucleic acids under conditions of high stringency. Only highly complementary nucleic acid hybrids form under conditions of high stringency. Accordingly, the stringency of the assay conditions determines the amount of complementarity that should exist between two nucleic acid strands in order to form a hybrid. Stringency should be chosen to maximize the difference in stability between the probe:target and probe:non-target hybrids.

Probes may be designed from the nucleic acids of the invention through methods known in the art. For instance, the G+C content of the probe and the probe length can affect probe binding to its target sequence. Methods to optimize probe specificity are commonly available in Sambrook et al., (1989) Molecular Cloning—A Laboratory Manual, Cold Spring Harbour Laboratory Press) or Ausubel et al., (1995) Current Protocols in Molecular Biology, Greene Publishing Co.

Hybridization conditions are modified using known methods, such as those described by Sambrook et al. and Ausubel et al. (supra) as required for each probe. Hybridization of total cellular RNA or RNA enriched for polyA RNA can be accomplished in any available format. For instance, total cellular RNA or RNA enriched for polyA RNA can be affixed to a solid support and the solid support exposed to at least one probe comprising at least one, or part of one of the sequences of the invention under conditions in which the probe will specifically hybridize. Alternatively, nucleic acid fragments comprising at least one, or part of one of the sequences of the invention can be affixed to a solid support, such as a silicon chip or a porous glass wafer. The solid support can then be exposed to total cellular RNA or polyA RNA from a sample under conditions in which the affixed sequences will specifically hybridize. Such solid supports and hybridization methods are widely available, for example, those disclosed by Beattie, (1995) WO 95/11755. By examining for the ability of a given probe to specifically hybridize to an RNA sample from an untreated cell population and from a cell population exposed to the agent, agents which up or down regulate the expression of a nucleic acid encoding the protein comprising the sequence of SEQ ID NO: 2 or 4 are identified.

Hybridization for qualitative and quantitative analysis of mRNAs may also be carried out by using an RNase Protection Assay (ie., RPA, see Ma et al. (1996) Methods 10, 273-238). Briefly, an expression vehicle comprising cDNA encoding the gene product and a phage specific DNA dependent RNA polymerase promoter (e.g., T7, T3 or SP6 RNA polymerase) is linearized at the 3′ end of the cDNA molecule, downstream from the phage promoter, wherein such a linearized molecule is subsequently used as a template for synthesis of a labeled antisense transcript of the cDNA by in vitro transcription. The labeled transcript is then hybridized to a mixture of isolated RNA (i.e., total or fractionated mRNA) by incubation at 45° C. overnight in a buffer comprising 80% formamide, 40 mM Pipes, pH 6.4, 0.4 M NaCl and 1 mM EDTA. The resulting hybrids are then digested in a buffer comprising 40 μg/ml ribonuclease A and 2 μg/ml ribonuclease. After deactivation and extraction of extraneous proteins, the samples are loaded onto urea polyacrylamide gels for analysis.

In another assay format, cells or cell lines are first identified which express the gene products of the invention physiologically, for example lung, kidney, intestine, heart and spleen cells. Cell and/or cell lines so identified would be expected to comprise the necessary cellular machinery such that the fidelity of modulation of the transcriptional apparatus is maintained with regard to exogenous contact of agent with appropriate surface transduction mechanisms and/or the cytosolic cascades. Further, such cells or cell lines would be transduced or transfected with an expression vehicle (e.g., a plasmid or viral vector) construct comprising an operable non-translated 5′-promoter containing end of the structural gene encoding the instant gene products fused to one or more antigenic fragments, which are peculiar to the instant gene products, wherein said fragments are under the transcriptional control of said promoter and are expressed as polypeptides whose molecular weight can be distinguished from the naturally occurring polypeptides or may further comprise an immunologically distinct tag or other detectable marker. Such a process is well known in the art (see Sambrook et al., (1989) Molecular Cloning—A Laboratory Manual, Cold Spring Harbour Laboratory Press).

Cells or cell lines transduced or transfected as outlined above are then contacted with agents under appropriate conditions; for example, the agent in a pharmaceutically acceptable excipient (vehicle) is contacted with cells in an aqueous physiological buffer such as phosphate buffered saline (PBS) at physiological pH, Eagles balanced salt solution (BSS) at physiological pH, PBS or BSS comprising serum or conditioned media comprising PBS or BSS and/or serum incubated at 37° C. Said conditions may be modulated as deemed necessary by one of skill in the art. Subsequent to contacting the cells with the agent, said cells will be disrupted and the polypeptides of the lysate are fractionated such that a polypeptide fraction is pooled and contacted with an antibody to be further processed by immunological assay (e.g., ELISA, immunoprecipitation or Western blot). The pool of proteins isolated from the “agent-contacted” sample will be compared with a control sample where only the excipient is contacted with the cells and an increase or decrease in the immunologically generated signal from the “agent-contacted” sample compared to the control will be used to distinguish the effectiveness of the agent.

J. Methods to Identify Agents that Modulate the Level or Activity of a Protein of the Invention

The invention provides methods for identifying agents that modulate the level or at least one activity of a protein of the invention such as the protein having the amino acid sequence of SEQ ID NO: 2 or 4. Such methods or assays may use any means of monitoring or detecting the desired activity.

In one format, the relative amounts of a protein of the invention between a cell population that has been exposed to the agent to be tested compared to an un-exposed control cell population may be assayed. In this format, probes such as specific antibodies are used to monitor the differential expression of the protein in the different cell populations. Cell lines or populations, such as lung cells or lung cell explants, are exposed to the agent to be tested under appropriate conditions and time. Qualitative or quantitative observations on changes in the gross morphology, histology, immunohistochemistry may be made on the agent-exposed and control explants or cells or sections thereof. Alternatively or in addition, cellular lysates may be prepared from the exposed cell line or population and a control, unexposed cell line or population. The cellular lysates are then analyzed with the probe.

Antibody probes are prepared by immunizing suitable mammalian hosts in appropriate immunization protocols using the peptides, polypeptides or proteins of the invention if they are of sufficient length, or, if desired, or if required to enhance immunogenicity, conjugated to suitable carriers. Suitable peptides include, for example, those encoded by amino acid residues 119-137, or 234-251 of SEQ ID NO: 2, or the corresponding residues of SEQ ID NO: 4. Methods for preparing immunogenic conjugates with carriers such as BSA, KLH, or other carrier proteins are well known in the art. In some circumstances, direct conjugation using, for example, carbodiimide reagents may be effective; in other instances linking reagents such as those supplied by Pierce Chemical Co. (Rockford, Ill.), may be desirable to provide accessibility to the hapten. The hapten peptides can be extended at either the amino or carboxy terminus with a cysteine residue or interspersed with cysteine residues, for example, to facilitate linking to a carrier. Administration of the immunogens is conducted generally by injection over a suitable time period and with use of suitable adjuvants, as is generally understood in the art. During the immunization schedule, titers of antibodies are taken to determine adequacy of antibody formation.

While the polyclonal antisera produced in this way may be satisfactory for some applications, for pharmaceutical compositions, monoclonal preparations may also be used. Immortalized cell lines which secrete the desired monoclonal antibodies may be prepared using the standard method of Kohler and Milstein (Nature (1975) 256:495-497) or modifications which effect immortalization of lymphocytes or spleen cells, as is generally known. The immortalized cell lines secreting the desired antibodies are screened by immunoassay in which the antigen is the peptide hapten, polypeptide or protein. When the appropriate immortalized cell culture secreting the desired antibody is identified, the cells can be cultured either in vitro or by production in ascites fluid.

The desired monoclonal antibodies are then recovered from the culture supernatant or from the ascites supernatant. Fragments of the monoclonal antibodies or the polyclonal antisera that contain the immunologically significant portion can be used as antagonists, as well as the intact antibodies. Immunologically reactive antibody fragments, such as the Fab, Fab′, or F(ab′)₂ fragments may be useful, especially in a therapeutic context, as these fragments are generally less immunogenic than the whole immunoglobulin.

The antibodies or antibody fragments of the invention may also be produced, using current technology, by recombinant means. Antibody regions that bind specifically to the desired regions of the protein can also be produced in the context of chimeras with multiple species origin, such as humanized antibodies.

In another assay format, one or more activities of a protein of the invention may be assayed in the presence and absence of an agent to be tested to determine whether they are capable of modulating (i.e. enhancing or inhibiting) this activity, for example alveolarization or branching morphogenesis in fetal lung or kidney explants expressing a protein of the invention. The ability of an agent to modulate branching morphogenesis may generally be evaluated in vitro by assaying the effect of the agent on cultured cells or explants wherein the cells under study are treated with a polypeptide of the invention or express a protein of the invention either endogenously or as a result of transfection with a nucleic acid of the invention. In general, an agent is considered to be a modulator of branching morphogenesis if, within one or more of these representative assays, contact of the test cells with the agent results in a discernible disruption of branching morphogenesis.

Agents that are assayed in the above method can be randomly selected or rationally selected or designed. As used herein, an agent is said to be randomly selected when the agent is chosen randomly without considering the specific sequences involved in the association of a protein of the invention alone or with its associated substrates, binding partners, etc. An example of randomly selected agents is the use a chemical library or a peptide combinatorial library, or a growth broth of an organism.

As used herein, an agent is said to be rationally selected or designed when the agent is chosen on a nonrandom basis which takes into account the sequence of the target site and/or its conformation in connection with the agent's action. Agents can be rationally selected or rationally designed by using the peptide sequences that make up these sites. For example, a rationally selected peptide agent can be a peptide whose amino acid sequence is identical to or a derivative of any functional consensus site.

The agents of the present invention can be, as examples, peptides, small molecules, vitamin derivatives, as well as carbohydrates. Dominant negative proteins, DNAs encoding these proteins, antibodies to these proteins, peptide fragments of these proteins or mimics of these proteins may be introduced into cells to affect function. The term “mimic” as used herein refers to the modification of a region or several regions of a peptide molecule to provide a structure chemically different from the parent peptide but topographically and functionally similar to the parent peptide (see Grant G A. in: Meyers (ed.) Molecular Biology and Biotechnology (New York, VCH Publishers, 1995), pp. 659-664; see also Section G). A skilled artisan can readily recognise that there is no limit as to the structural nature of the agents of the present invention.

The peptide agents of the invention can be prepared using standard solid phase (or solution phase) peptide synthesis methods, as is known in the art. In addition, the DNA encoding these peptides may be synthesized using commercially available oligonucleotide synthesis instrumentation and produced recombinantly using standard recombinant production systems. The production using solid phase peptide synthesis is necessitated if non-gene-encoded amino acids are to be included.

Another class of agents of the present invention are antibodies that are immunoreactive with critical positions of proteins of the invention. Antibody agents are obtained by immunization of suitable mammalian subjects with peptides containing, as antigenic regions, those portions of the protein to be targeted by the antibodies.

K. Uses for Agents that Modulate the Level or Activity of a Protein of the Invention

As provided in the Examples, the proteins and nucleic acids of the invention, such as the protein having the amino acid sequence of SEQ ID NO: 2 or 4, modulate branching morphogenesis and alveolarization. Agents that up-or-down-regulate or modulate the expression of the protein, or modulate at least one activity of the protein, such as agonists or antagonists or other inhibitors, may be used to modulate biological and pathologic processes associated with the protein's function and activity.

As used herein, a subject can be any mammal, so long as the mammal is in need of modulation of a pathological or biological process mediated by a protein of the invention. The term “mammal” is defined as an individual belonging to the class Mammalia. The invention is particularly useful in the treatment of human subjects. Pathological processes refer to a category of biological processes that produce a deleterious effect. As used herein, an agent is said to modulate a pathological process when the agent alters the degree or severity of the process.

For example, underexpression of a protein of the invention may be associated with hypoplasia or abnormal morphogenesis of the lung, kidney, intestine, heart, spleen or other tissue where LGL1 is expressed, or abnormal alveolarization. Abnormalities may be qualitative or quantitative abnormalities and the defects of abnormal alveolarization may be due to primary failure of alveolar formation or to inadequate tissue repair in disease, e.g. pursuant to destruction of alveoli. Kidney disorders such as Polycystic Kidney Disease (PKD) and lung disorders such as BPD, New BPD, CDH, COPD, chronic bronchial infection or emphysema, and disorders related to abnormal branching and/or abnormal alveolarization may be prevented or disease progression modulated by the administration of agents which up- or down-regulate or modulate in some way the expression or at least one activity of a protein of the invention.

The agents of the present invention can be provided alone, or in combination with other agents that modulate a particular pathological process. For example, an agent of the present invention can be administered in combination with other known drugs. As used herein, two agents are said to be administered in combination when the two agents are administered simultaneously or are administered independently in a fashion such that the agents will act at the same time.

Administration of the agents of the present invention can be parenteral, subcutaneous, intravenous, intramuscular, intraperitoneal, transdermal, buccal or pulmonary (e.g., by inhalation or insufflation of powders or aerosols, including by nebulizer; intratracheal or intranasal). Alternatively, or concurrently, administration may be by the oral route. The dosage administered will be dependent upon the age, health, and weight of the recipient, kind of concurrent treatment, if any, frequency of treatment, and the nature of the effect desired.

The present invention further provides compositions containing one or more agents that modulate expression or at least one activity of a protein of the invention. While individual needs vary, determination of optimal ranges of effective amounts of each component is within the skill of the art. Typical dosages comprise about 0.1 to 100 μg/kg body wt, about 0.1 to 10 μg/kg body wt, or about 0.1 to 1 μg/kg body wt. Other aspects of the formulation and delivery of a composition may also be determined without undue experimentation, for example aerosol particle size and inspiration flow rate.

In addition to the pharmacologically active agent, the compositions of the present invention may contain suitable pharmaceutically acceptable carriers comprising excipients (vehicles) and auxiliaries that facilitate processing of the active compounds into preparations which can be used pharmaceutically for delivery to the site of action. Suitable formulations for parenteral administration include aqueous solutions of the active compounds in water-soluble form, for example, water-soluble salts. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances which increase the viscosity of the suspension include, for example, sodium carboxymethyl cellulose, sorbitol, and/or dextran. Optionally, the suspension may also contain stabilizers. Liposornes can also be used to encapsulate the agent for delivery into the cell.

The pharmaceutical formulation for systemic administration according to the invention may be formulated for enteral, parenteral or topical administration. Indeed, all three types of formulations may be used simultaneously to achieve systemic administration of the active ingredient. Standard formulations for pulmonary (aerosol) administration include sterile, non-pyrogenic aqueous solutions. Suitable formulations for oral administration include hard or soft gelatin capsules, pills, tablets, including coated tablets, elixirs, suspensions, syrups or inhalations and controlled release forms thereof.

In practising the methods of this invention, the agents, compounds and compositions of this invention may be used alone or in combination with other therapeutic or diagnostic agents and procedures. In an embodiment of the invention, the agents, compounds and compositions of this invention may be co-administered along with other compounds or procedures typically prescribed for these conditions according to generally accepted medical practice. For example, current product offerings for emphysema include but are not limited to bronchodilators, anticholinergics, steroids, oxygen therapy, and aerosolized surfactant, and in the case of pediatric disorders current therapies include but are not limited to positive pressure ventilation and supplemental oxygen.

The agents, compounds and compositions of this invention can be utilised in vivo, ordinarily in mammals, such as humans, sheep, horses, cattle, pigs, dogs, cats, rats and mice, or in vitro.

L. Transgenic Animals

Transgenic animals containing mutant, knock-out or modified genes corresponding to the cDNA sequence of SEQ ID NO: 1 or 3, or the open reading frame encoding the polypeptide sequence of SEQ ID NO: 2 or 4 or fragments thereof having a consecutive sequence of at least about 3, 4, 5, 6, 7, 10, 15, 20, 25, 30, 35, 70, 100, 150, 200, 300, 400 or more amino acid residues, are also included in the invention. Transgenic animals are genetically modified animals into which recombinant, exogenous or cloned genetic material has been experimentally transferred. Such genetic material is often referred to as a “transgene.” The nucleic acid sequence of the transgene, in this case a form of SEQ ID NO: 1 or 3, may be integrated either at a locus of a genome where that particular nucleic acid sequence is not otherwise normally found or at the normal locus for the transgene. The transgene may consist of nucleic acid sequences derived from the genome of the same species or of a different species than the species of the target animal.

The term “germ cell line transgenic animal” refers to a transgenic animal in which the genetic alteration or genetic information was introduced into a germ line cell, thereby conferring the ability of the transgenic animal to transfer the genetic information to offspring. If such offspring in fact possess some or all of that alteration or genetic information, then they too are transgenic animals.

The alteration or genetic information may be foreign to the species of animal to which the recipient belongs, foreign only to the particular individual recipient, or may be genetic information already possessed by the recipient. In the last case, the altered or introduced gene may be expressed differently than the native gene.

Transgenic animals can be produced by a variety of different methods including transfection, electroporation, microinjection, gene targeting in embryonic stem cells and recombinant viral and retroviral infection (see, e.g., U.S. Pat. No. 4,736,866; U.S. Pat. No. 5,602,307; Mullins et al., (1993) Hypertension 22, 630-633; Brenin et al., (1997) Surg. Oncol. 6, 99-110; Tuan (1997) Recombinant Gene Expression Protocols, Methods in Molecular Biology, Humana Press).

A number of recombinant or transgenic mice have been produced, including those which express an activated oncogene sequence (U.S. Pat. No. 4,736,866); express simian SV40 T-antigen (U.S. Pat. No. 5,728,915); lack the expression of interferon regulatory factor 1 (IRF-1) (U.S. Pat. No. 5,731,490); exhibit dopaminergic dysfunction (U.S. Pat. No. 5,723,719); express at least one human gene which participates in blood pressure control (U.S. Pat. No. 5,731,489); display greater similarity to the conditions existing in naturally occurring Alzheimer's disease (U.S. Pat. No. 5,720,936); have a reduced capacity to mediate cellular adhesion (U.S. Pat. No. 5,602,307); possess a bovine growth hormone gene (Clutter et al., (1996) Genetics 143, 1753-1760); or, are capable of generating a fully human antibody response (McCarthy (1997) Lancet 349, 405).

While mice and rats remain the animals of choice for most transgenic experimentation, in some instances it may be decided or even necessary to use alternative animal species. Transgenic procedures have been successfully utilised in a variety of non-murine animals, including sheep, goats, pigs, dogs, cats, monkeys, chimpanzees, hamsters, rabbits, cows and guinea pigs (see, e.g., Kim et al., (1997) Mol. Reprod. Dev. 46, 515-526; Houdebine (1995) Reprod. Nutr. Dev. 35, 609-617; Petters (1994) Reprod. Fertil. Dev. 6, 643-645; Schnieke et al., (1997) Science 278, 2130-2133; and Amoah (1997) J. Animal Science 75, 578-585).

The method of introduction of nucleic acid fragments into recombination competent mammalian cells can be by any method that favours co-transforrnation of multiple nucleic acid molecules. Detailed procedures for producing transgenic animals are readily available to one skilled in the art, including the disclosures in U.S. Pat. No. 5,489,743 and U.S. Pat. No. 5,602,307.

M. Diagnostic Methods

This invention also provides for diagnostic methods using an LGL gene as part of a diagnostic assay for detecting diseases or susceptibility to diseases, for example, disease related to the presence of mutations in the LGL1 nucleic acid sequences.

Mutations in the LGL1 gene may be detected at the DNA level by a variety of techniques. Nucleic acids for diagnosis may be obtained from a patient's cells, such as from blood, urine, saliva, tissue biopsy and autopsy material. The genomic DNA may be used directly for detection or may be amplified enzymatically by using PCR (Saiki et al., Nature 324:163-166 (1986)) prior to analysis. RNA or cDNA may also be used for the same purpose. As an example, PCR primers complementary to the nucleic acid encoding the nucleotide of the invention can be used to identify and analyze mutations. For example, deletions and insertions can be detected by a change in size of the amplified product in comparison to the normal genotype. Point mutations can be identified by hybridizing amplified DNA to radiolabeled LGL1 RNA or alternatively, to radiolabeled LGL1 antisense DNA sequences. Perfectly matched sequences can be distinguished from mismatched duplexes by RNase A digestion or by differences in melting temperatures.

Genetic testing based on DNA sequence differences may be achieved by detection of alteration in electrophoretic mobility of DNA fragments in gels with or without denaturing agents. Small sequence deletions and insertions can be visualized by high resolution gel electrophoresis. DNA fragments of different sequences may be distinguished on denaturing formamide gradient gels in which the mobilities of different

DNA fragments are retarded in the gel at different positions according to their specific melting or partial melting temperatures (see, e.g., Myers et al., Science, 230:1242 (1985)). Sequence changes at specific locations may also be revealed by nuclease protection assays such as RNase and SI protection or the chemical cleavage method (e.g., Cotton et al., PNAS, USA, 85:4397-4401 (1985)). Thus, the detection of a specific DNA sequence may be achieved by methods such as hybridization, RNase protection, chemical cleavage, direct DNA sequencing or the use of restriction enzymes, (e.g., Restriction Fragment Length Polymorphisms (RFLP)) and Southern blotting of genomic DNA. In addition to more conventional gel-electrophoresis and DNA sequencing, mutations can also be detected by in situ analysis.

The present invention provides for diagnostic assays for detecting altered levels of the nucleic acid molecules or polypeptides of the invention in various tissues. An under- or over-expression of the polypeptides compared to normal control tissue samples may detect the presence or stage of a disease or susceptibility to a disease, for example, dysplasia such as hypoplasia or hyperplasia, or a tumour. As the genes and proteins of the invention are expressed in fetal lung cells known to support lung epithelial cell development, and as this expression occurs during the developmental stages involving branching morphogenesis and alveolarization, the genes and proteins of the invention may be used to diagnose or monitor lung disorders involving quantitatively or qualitatively abnormal morphogenesis or quantitatively or qualitatively abnormal alveolarization, lung function, other lung disorders or to track disease progression.

One means of diagnosing lung disorders using the nucleic acid molecules or proteins of the invention involves obtaining lung cells or tissue from living subjects. Respiratory cell samples can be obtained by bronchoalveolar lavage, bronchoscopy and scraping of respiratory cells. Obtaining tissue samples from living sources is problematic for tissues such as lung. However, due to the nature of the treatment paradigms for lung disorders, biopsy may be necessary. Further, it may be desirable to obtain biopsy samples from different regions of the lung for analysis. Lung tissues can also be obtained at autopsy for post-mortem diagnoses. When possible, urine, blood or peripheral lymphocyte samples may be used as the tissue sample in the assay. Methods of the invention may involve treatment of tissues with collagenases or other proteases to make the tissue amenable to cell lysis (Semenov D E et al., (1987) Biull Eksp Biol Med 104, 113-116).

The use of molecular biological tools has become routine in forensic technology. For example, nucleic acid probes comprising all or at least part of the sequence of SEQ ID NO: 1 or 3 may be used to determine the expression of a nucleic acid molecule in forensic/pathology specimens. Further, nucleic acid assays may be carried out by any means of conducting a transcriptional profiling analysis. In addition to nucleic acid analysis, forensic methods of the invention may target the proteins of the invention, particularly a protein comprising SEQ ID NO: 2 or 4, to determine up or down regulation of the genes (Shiverick et al., (1975) Biochim Biophys Acta 393, 124-133).

Assays to detect nucleic acid or protein molecules of the invention may be in any available format. Typical assays for nucleic acid molecules include hybridization or PCR based formats. Assays used to detect levels of protein in a sample derived from a host are well known to those of skill in the art and include radioimmunoassays, competitive-binding assays, Western Blot analysis, ELISA assays and “sandwich” assay. Typical assays include the use of antibody probes in any available format such as in situ binding assays, etc. See Harlow & Lane, (1988) Antibodies—A Laboratory Manual, Cold Spring Harbor Laboratory Press. Typically, assays incorporate appropriate controls.

The above methods may also be used in other diagnostic protocols, including protocols and methods to detect disease states in other tissues or organs, for example the tissues in which gene expression is detected, such as the kidney, heart, spleen and intestine.

N. Applications of LGL1

The invention also provides for methods of use of the lgl1 polypeptides, variants or biologically active fragments thereof, for modulating an activity of the polynucleotide or polypeptide of the invention in animals, such as mammals, including humans. In an embodiment of the invention lgl1 polypeptides or fragments thereof are used for the treatment of or amelioration of symptoms in any lung disease, condition or disorder where airway branching and/or alveolarization would be beneficial. Lung diseases, conditions or disorders in which the lgl1 polypeptides or fragments or derivatives thereof and pharmaceutical compositions of the present invention can be used include, but are not limited to emphysema, COPD, CDH, BPD and New BPD.

The methods of the present invention comprise administering to a subject in need thereof an effective amount of an lgl1 polypeptide or variant or biologically active fragment thereof, or a composition comprising an lgl1 polypeptide or variant or biologically active fragment thereof, to a subject. In one embodiment, an effective amount of a therapeutic composition comprising an lgl1 polypeptide or variant or biologically active fragment thereof, and a pharmaceutical carrier is administered to a subject.

For lung treatment, pulmonary administration methods include inhalation or insufflation of powders or aerosols, including by nebulizer; intratracheal, and intranasal routes. Furthermore, lgl1 polypeptides or variants or biologically active fragments thereof, can be expressed by administration of lgl1- or lgl1 fragment-encoding cDNA, plasmid, liposomes, viral vectors (HSV or adenoviral), or transformed cells e.g. fibroblasts, or released by encapsulated cells, etc.

In different embodiments of the present invention the lgl1 polypeptide or variant or biologically active fragment thereof, or pharmaceutical composition of the present invention is administered before, after or during administration of other treatments for lung disease.

O. Antisense Compounds

The antisense oligonucleotide compounds of the invention makes them useful in diagnostics, therapeutics, prophylaxis and as research reagents and kits. In the context of this invention, the term “antisense compound” or “antisense oligonucleotide” refers to an oligomer or polymer of ribonucleic acid (RNA) or deoxyribonucleic acid (DNA) or mimetics thereof. This term includes oligonucleotides composed of naturally occurring nucleobases, sugars and covalent internucleoside (backbone) linkages as well as oligonucleotides having non-naturally-occurring moieties that function similarly. Such modified or substituted oligonucleotides are well known to workers skilled in the art and often chosen over native forms because of desirable properties such as, for example, enhanced cellular uptake, enhanced affinity for nucleic acid target and increased stability in the presence of nucleases.

Specific examples of antisense compounds of the invention include oligonucleotides containing modified backbones or non-natural internucleoside linkages. Antisense oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-240 . Various salts, mixed salts and free acid forms are also included.

Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253, 5,571,799; 5,587,361; and 5,625,050.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones, formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

In other oligonucleotide mimetics, both the sugar and the intemucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262. Further teaching of PNA compounds can be found in Nielsen et al., Science, 1991, 254, 1497-1500.

Embodiments of the invention include oligonucleotides with phosphorothioate backbones and oligonucleosides with heteroatom backbones, and in particular —CH₂—NH—O—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂—and —O—N(CH₃)—CH₂—CH₂— [wherein the native phosphodiester backbone is represented as —O—P—O—CH₂—] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

Modified oligonucleotides may also contain one or more substituted sugar moieties. Preferred oligonucleotides comprise one of the following at the 2′ position: OH; F; O—, S—, or N-alkyl; O—, S—, or N-alkenyl; O—, S— or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘ CH₃, O(CH₂)ₙOCH₃, O(CH₂)ₙ NH₂, O(CH₂)ₙ CH₃, O(CH₂)ₙ ONH₂, and O(CH₂)ₙ ON[(CH₂)ₙ CH₃)]₂, where n and m are from 1 to about 10. Other preferred oligonucleotides comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the phannacokinetic properties of an oligonucleotide, or a group for improving the pharmacodynamic properties of an oligonucleotide, and other substituents having similar properties. A preferred modification includes 2′-methoxyethoxy (2′-O—CH₂ CH₂ OCH₃, also known as 2′-O-(2-methoxyethyl) or 2′-MOE) (Martin et al., Helv. Chim. Acta, 1995, 78, 486-504) i.e., an alkoxyalkoxy group. A further preferred modification includes 2′-dimethylaminooxyethoxy, i.e., a O(CH₂)₂ ON(CH₃)₂ group, also known as 2′-DMAOE, as described in examples hereinbelow.

Other antisense oligonucleotides of the invention include include 2′-methoxy (2′-O—CH₃), 2′-aminopropoxy (2′-OCH₂CH₂CH₂NH₂) and 2′-fluoro (2′-F) modifications. Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920.

Oligonucleotides may also include nucleobase (often referred to in the art simply as “base”) modifications or substitutions. As used herein, “unmodified”or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of the oligomeric compounds of the invention. These include 5-substituted pyrinidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278) and are presently preferred base substitutions, even more particularly when combined with 2′-O-methoxyethyl sugar modifications.

Representative United States patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941, and 5,750,692.

Another modification of the oligonucleotides of the invention involves chemically linking to the oligonucleotide one or more moieties or conjugates which enhance the activity, cellular distribution or cellular uptake of the oligonucleotide. Such moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Lett.,1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamnine or hexylamino-carbonyl-oxycholesterol moiety (Crooke et al., J. Pharmacol. Exp. Ther., 1996, 277, 923-937.

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

It is not necessary for all positions in a given compound to be uniformly modified, and in fact more than one of the aforementioned modifications may be incorporated in a single compound or even at a single nucleoside within an oligonucleotide. The present invention also includes antisense compounds which are chimeric compounds. “Chimeric” antisense compounds or “chimeras,” in the context of this invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense compounds of the invention may be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, certain of which are commonly owned with the instant application, and each of which is herein incorporated by reference in its entirety.

The antisense compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

The antisense compounds of the invention are synthesized in vitro and do not include antisense compositions of biological origin, or genetic vector constructs designed to direct the in vivo synthesis of antisense molecules. The compounds of the invention may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Representative United States patents that teach the preparation of such uptake, distribution and/or absorption assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756.

The antisense compounds of the invention are useful as research reagents and in diagnostics. For example, antisense oligonucleotides that are able to inhibit gene expression with exquisite specificity, can be used by those of ordinary skill in the art to elucidate the function of particular genes, or to distinguish between functions of various members of a biological pathway. In diagnostic uses, hybridization of the antisense oligonucleotides of the invention with a nucleic acid encoding LGL1 can be detected by means known in the art. Such means may include conjugation of an enzyme to the oligonucleotide, radiolabelling of the oligonucleotide or any other suitable detection means. Kits using such detection means for detecting the level of LGL1 in a sample may also be prepared.

Antisense oligonucleotides can be used as therapeutic moieties in the treatment of disease states. For therapeutics, an animal, such as a human, suspected of having a disease or disorder which can be treated by modulating the expression of LGL1 is treated by administering antisense compounds in accordance with this invention. The antisense compounds of the invention can be used in pharmaceutical compositions by adding an effective amount of an antisense compound to a suitable pharmaceutically acceptable diluent or carrier. Use of the antisense compounds and methods of the invention may also be useful prophylactically.

The present invention also includes pharmaceutical compositions and formulations that include the antisense compounds of the invention. The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Oligonucleotides with at least one 2′-O-methoxyethyl modification are believed to be particularly useful for oral administration.

Antisense Targets

Specific nucleic acids may be targeted for antisense use. “Targeting” an antisense compound to a particular nucleic acid, in the context of this invention, is a multistep process. The process usually begins with the identification of a nucleic acid sequence whose function is to be modulated. This may be, for example, a cellular gene (or mRNA transcribed from the gene) whose expression is associated with a particular disorder or disease state, or a nucleic acid molecule from an infectious agent. In the present invention, the target is a nucleic acid molecule encoding LGL1. The targeting process also includes determination of a site or sites within this gene for the antisense interaction to occur such that the desired effect, e.g., detection or modulation of expression of the protein, will result. Within the context of the present invention, a preferred intragenic site is the region encompassing the translation initiation or termination codon of the open reading frame (ORF) of the gene. Since, as is known in the art, the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). It is also known in the art that eukaryotic and prokaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of an mRNA molecule transcribed from a gene encoding LGL1, regardless of the sequence(s) of such codons.

It is also known in the art that a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon. The open reading frame (ORF) or “coding region,” which is known in the art to refer to the region between the translation initiation codon and the translation termination codon, is also a region which may be targeted effectively. Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′UTR), known in the art to refer to the portion of an mRNA in the 3′ direction from the translation termination codon, and thus including nucleotides between the translation termination codon and 3′ end of an mRNA or corresponding nucleotides on the gene. The 5′ cap of an mRNA comprises an N⁷-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap. The 5′ cap region may also be a preferred target region.

Although some eukaryotic mRNA transcripts are directly translated, many contain one or more regions, known as “introns,” which are excised from a transcript before it is translated. The remaining (and therefore translated) regions are known as “exons” and are spliced together to form a continuous mRNA sequence. mRNA splice sites, i.e., intron-exon junctions, may also be preferred target regions, and are particularly useful in situations where aberrant splicing is implicated in disease, or where an overproduction of a particular mRNA splice product is implicated in disease. Aberrant fusion junctions due to rearrangements or deletions are also preferred targets. It has also been found that introns can also be effective, and therefore preferred, target regions for antisense compounds targeted, for example, to DNA or pre-mRNA.

Once one or more target sites have been identified, oligonucleotides are chosen which are sufficiently complementary to the target, i.e., hybridize sufficiently well and with sufficient specificity, to give the desired effect. In the context of this invention, “hybridization” means hydrogen bonding, which may be Watson-Crick, Hoogsteen or reversed Hoogsteen hydrogen bonding, between complementary nucleoside or nucleotide bases. For example, adenine and thymine are complementary nucleobases that pair through the formation of hydrogen bonds. “Complementary,” as used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the oligonucleotide and the DNA or RNA target.

It is understood in the art that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired, i.e., under physiological conditions in the case of in vivo assays or therapeutic treatment, and in the case of in vitro assays, under conditions in which the assays are performed.

P. Gene Therapy

The polypeptides of the invention or fragments thereof may also be employed in accordance with the present invention by expression of such proteins in vivo, which is often referred to as “gene therapy.”

A lgl1 modulating amount of an expressible nucleic acid of the invention may be in the form of a gene therapy vector, or the proteins of the invention themselves, including fragments, analogues or derivatives thereof. The gene therapy constructs or compounds of the invention are administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including but not limited improved survival rate or more rapid recovery, or improvement or elimination of symptoms and other indicators as are selected as appropriate measures by those skilled in the art.

By gene therapy as used herein refers to the transfer of genetic material (e.g DNA or RNA) of interest into a host to treat or prevent a genetic or acquired disease or condition phenotype. The genetic material of interest encodes a product (e.g. a protein, polypeptide, peptide or functional RNA) whose production in vivo is desired. For example, the genetic material of interest can encode a hormone, receptor, enzyme, polypeptide or peptide of therapeutic value. For a review see, in general, the text “Gene Therapy” (Advances in Pharmacology 40, Academic Press, 1997).

Two basic approaches to gene therapy have evolved: (1) ex vivo and (2) in vivo gene therapy. In ex vivo gene therapy cells are removed from a patient, and while being cultured are treated in vitro. Generally, a functional replacement gene is introduced into the cell via an appropriate gene delivery vehicle/method (transfection, transduction, homologous recombination, etc.) and an expression system as needed and then the modified cells are expanded in culture and returned to the host/patient. These genetically reimplanted cells have been shown to produce the transfected gene product in situ.

In in vivo gene therapy, target cells are not removed from the subject rather the gene to be transferred is introduced into the cells of the recipient organism in situ, that is within the recipient. Alternatively, if the host gene is defective, the gene is repaired in situ [Culver, 1998]. These genetically altered cells have been shown to produce the transfected gene product in situ.

The gene expression vehicle is capable of delivery/transfer of heterologous nucleic acid into a host cell. The expression vehicle may include elements to control targeting, expression and transcription of the nucleic acid in a cell selective manner as is known in the art. It should be noted that often the 5′UTR and/or 3′UTR of the gene may be replaced by the 5′UTR and/or 3′UTR of the expression vehicle. Therefore as used herein the expression vehicle may, as needed, not include the 5′UTR and/or 3′UTR shown in SEQ ID No: 1 and only include the specific amino acid coding region for R1, an R1 peptide or this coding region may be modified to produce an R1 analog.

The expression vehicle can include a promotor for controlling transcription of the heterologous material and can be either a constitutive or inducible promotor to allow selective transcription. Enhancers that may be required to obtain necessary transcription levels can optionally be included. Enhancers are generally any non-translated DNA sequence which works contiguously with the coding sequence (in cis) to change the basal transcription level dictated by the promoter. The expression vehicle can also include a selection gene as described herein below.

Vectors are one means of a gene delivery vehicle and can be introduced into cells or tissues by any one of a variety of known methods within the art. Such methods can be found generally described in Sambrook et al., Molecular Cloning: A Laboratory Manual, Cold Springs Harbor Laboratory, New York (1989, 1992), in Ausubel et al., Current Protocols in Molecular Biolog, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor, Mich. (1995), Vectors: A Survey of Molecular Cloning Vectors and Their Uses, Butterworths, Boston Mass. (1988) and Gilboa et al (1986) and include, for example, stable or transient transfection, lipofection, electroporation and infection with recombinant viral vectors. In addition, see U.S. Pat. No. 4,866,042 for vectors involving the central nervous system and also U.S. Pat. No. 5,464,764 and 5,487,992 for positive-negative selection methods as well see U.S. Pat. Nos. 5,698,443; 5,686,278; 5,538,885; 5,691,176; 5,585,254; 5,614,396; 5,670,488; 5,599,712; 5,645,829; 5,641,680 and 5,688,773.

Introduction of nucleic acids by infection offers several advantages over the other listed methods. Higher efficiency can be obtained due to their infectious nature. Moreover, viruses are very specialized and typically infect and propagate in specific cell types. Thus, their natural specificity can be used to target the vectors to specific cell types in vivo or within a tissue or mixed culture of cells. Viral vectors can also be modified with specific receptors or ligands to alter target specificity through receptor mediated events.

A specific example of DNA viral vector for introducing and expressing recombinant sequences is the adenovirus derived vector Adenop53TK. This vector expresses a herpes virus thymidine kinase (TK) gene for either positive or negative selection and an expression cassette for desired recombinant sequences. This vector can be used to infect cells that have an adenovirus receptor, which includes most cancers of epithelial origin as well as others. This vector as well as others that exhibit similar desired functions can be used to treat a mixed population of cells and can include, for example, an in vitro or ex vivo culture of cells, a tissue or a human subject (see for example U.S. Pat. Nos. 5,691,176; 5,585,254; 5,670,488; 5,681,731)

Additional features can be added to the vector to ensure its safety and/or enhance its therapeutic efficacy. Such features include, for example, markers that can be used to negatively select against cells infected with the recombinant virus. An example of such a negative selection marker is the TK gene described above that confers sensitivity to the antibiotic gancyclovir. Negative selection is therefore a means by which infection can be controlled because it provides inducible suicide through the addition of an antibiotic. In addition, recombinant viral vectors are useful for in vivo expression of a desired nucleic acid because they offer advantages such as lateral infection and targeting specificity. Lateral infection is inherent in the life cycle of, for example, retroviruses and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. The vector to be used in the methods of the invention will depend on desired cell type to be targeted and will be known to those skilled in the art. For example, if breast cancer is to be treated then a vector specific for such epithelial cells would be used. Likewise, if diseases or pathological conditions of the hematopoietic system are to be treated, then a viral vector that is specific for blood cells and their precursors, preferably for the specific type of hematopoietic cell, would be used.

Retroviral vectors can be constructed to function either as infectious particles or to undergo only a single initial round of infection. In the former case, the genome of the virus is modified so that it maintains all the necessary genes, regulatory sequences and packaging signals to synthesize new viral proteins and RNA. Once these molecules are synthesized, the host cell packages the RNA into new viral particles which are capable of undergoing further rounds of infection. The vector's genome is also engineered to encode and express the desired recombinant gene. In the case of non-infectious viral vectors, the vector genome is usually mutated to destroy the viral packaging signal that is required to encapsulate the RNA into viral particles. Without such a signal, any particles that are formed will not proceed through subsequent rounds of infection. The specific type of vector will depend upon the intended application. The actual vectors are also known and readily available within the art or can be constructed by one skilled in the art using well-known methodology.

The recombinant vector can be administered in several ways. If viral vectors are used, for example, the procedure can take advantage of their target specificity and consequently, do not have to be administered locally at the diseased site. However, local administration can provide a quicker and more effective treatment, administration can also be performed by, for example, intravenous or subcutaneous injection into the subject. Injection of the viral vectors into a spinal fluid can also be used as a mode of administration, especially in the case of neuro-degenerative diseases. Following injection, the viral vectors will circulate until they recognize host cells with the appropriate target specificity for infection.

An alternate mode of administration can be by direct inoculation locally at the site of the disease or pathological condition or by inoculation into the vascular system supplying the site with nutrients or into the spinal fluid. Local administration is advantageous because there is no dilution effect and, therefore, a smaller dose is required to achieve expression in a majority of the targeted cells. Additionally, local inoculation can alleviate the targeting requirement needed with other forms of administration since a vector can be used that infects all cells in the inoculated area. If expression is desired in only a specific subset of cells within the inoculated area, then promoter and regulatory elements that are specific for the desired subset can be used to accomplish this goal. Such non-targeting vectors can be, for example, viral vectors, viral genome, plasmids, phagemids and the like. Transfection vehicles such as liposomes can also be used to introduce the non-viral vectors described above into recipient cells within the inoculated area. Such transfection vehicles are known by one skilled within the art.

Thus, for example, cells from a patient may be engineered with a polynucleotide (DNA or RNA) encoding a polypeptide ex vivo, with the engineered cells then being provided to a patient to be treated with the polypeptide. Such methods are well-known in the art. For example, cells may be engineered by procedures known in the art by use of a retroviral particle containing RNA encoding the lgl1 polypeptide or fragment according to the present invention.

Similarly, cells may be engineered in vivo for expression of a polypeptide in vivo by, for example, procedures known in the art. As known in the art, a producer cell for producing a retroviral particle containing RNA encoding an lgl1 polypeptide or fragment according to the present invention, may be administered to a patient for engineering cells in vivo and expression of the polypeptide in vivo. These and other methods for administering an lgl1 polypeptide or fragment according to the present invention, by such method should be apparent to those skilled in the art from the teachings of the present invention. For example, the expression vehicle for engineering cells may be other than a retrovirus, for example, an adenovirus which may be used to engineer cells in vivo after combination with a suitable delivery vehicle.

Retroviruses, from which the retroviral plasmid vectors hereinabove mentioned, may be derived include, but are not limited to, Moloney Murine Leukemia Virus, spleen necrosis virus, retroviruses such as Rous Sarcoma Virus, Harvey Sarcoma Virus, avian leukosis virus, gibbon ape leukemia virus, human immunodeficiency virus, adenovirus, Myeloproliferative Sarcoma Virus, and mammary tumour virus. In one embodiment, the retroviral plasmid vector is derived from Moloney Murine Leukemia Virus.

The vector includes one or more promoters. Suitable promoters which may be employed include, but are not limited to, the retroviral LTR; the SV40 promoter; and the human cytomegalovirus (CMV) promoter described in Miller, et al., Biotechniques, Vol. 7, No. 9, 980-990 (1989), or any other promoter (e.g., cellular promoters such as eukaryotic cellular promoters including, but not limited to, the histone, pol III, and β-actin promoters). Other viral promoters which may be employed include, but are not limited to, adenovirus promoters, thymidine kinase (TK) promoters, and B19 parvovirus promoters. The selection of a suitable promoter will be apparent to those skilled in the art from the teachings contained herein.

The nucleic acid sequence encoding the polypeptide of the present invention is under the control of a suitable promoter. Suitable promoters which may be employed include, but are not limited to, adenoviral promoters, such as the adenoviral major late promoter; or hetorologous promoters, such as the cytomegalovirus (CMV) promoter; the respiratory syncytial virus (RSV) promoter; inducible promoters, such as the MMT promoter, the metallothionein promoter; heat shock promoters; the albumin promoter; the ApoAI promoter; human globin promoters; viral thymidine kinase promoters, such as the Herpes Simplex thymidine kinase promoter; retroviral LTRs (including the modified retroviral LTRs hereinabove described); the β-actin promoter; and human growth hormone promoters. The promoter also may be the native promoter that controls the genes encoding the polypeptides.

The retroviral plasmid vector is employed to transduce packaging cell lines to form producer cell lines. Examples of packaging cells which may be transfected include, but are not limited to, the PE501, PA317, ψ-2, ψ-AM, PA12, T19-14X, VT-19-17-H2, ψCRE, ψCRIP, GP+E-86, GP+envAml12, and DAN cell lines as described in Miller, Human Gene Therapy, Vol. 1, pgs. 5-14 (1990), which is incorporated herein by reference in its entirety. The vector may transduce the packaging cells through any means known in the art. Such means include, but are not limited to, electroporation, the use of liposomes, and CaPO₄ precipitation. In one alternative, the retroviral plasmid vector may be encapsulated into a liposome, or PTH to a lipid, and then administered to a host.

The producer cell line generates infectious retroviral vector particles that include the nucleic acid sequence(s) encoding the polypeptides. Such retroviral vector particles then may be employed, to transduce eukaryotic cells, either in vitro or in vivo. The transduced eukaryotic cells will express the nucleic acid sequence(s) encoding the polypeptide. Eukaryotic cells which may be transduced include, but are not limited to, embryonic stem cells, embryonic carcinoma cells, as well as hematopoietic stem cells, hepatocytes, fibroblasts, myoblasts, keratinocytes, endothelial cells, and bronchial epithelial cells.

Q. Pharmaceutical Formulations

The present invention is also directed to therapeutic or pharmaceutical compositions comprising a pharmaceutically acceptable carrier and a protein of the invention, for example an lgl1 polypeptide, or fragment or derivative thereof. In one embodiment, the composition contains a recombinant lgl1 polypeptide as the active ingredient. In another embodiment the composition contains a nucleic acid encoding a protein of the invention.

The amount of the therapeutic or pharmaceutical composition of the invention which is effective in the treatment of a particular disease, condition or disorder will depend on the nature of the disease, condition or disorder and can be determined by standard clinical techniques. In vitro assays may optionally be employed to help identify optimal dosage ranges. The precise dose to be employed in the formulation will also depend on the seriousness of the disease, condition or disorder, and can be readily determined according to methods known to one skilled in the art. Effective doses may be extrapolated from dose-response curves derived from in vitro or animal model test systems.

The pharmaceutical compositions of the present invention may be administered orally, topically, parenterally, by inhalation or spray or rectally in dosage unit formulations containing conventional non-toxic pharmaceutically acceptable carriers, adjuvants and vehicles. The term parenteral as used herein includes subcutaneous injections, intravenous, intramuscular, intrasternal injection or infusion techniques. One or more protease inhibitor may be present in association with one or more non-toxic pharmaceutically acceptable carriers and/or diluents and/or adjuvants and, if desired, other active ingredients. The pharmaceutical compositions containing one or more protease inhibitor may be in a form suitable for oral use, for example, as tablets, troches, lozenges, aqueous or oily suspensions, dispersible powders or granules, emulsion hard or soft capsules, or syrups or elixirs.

Compositions intended for oral use may be prepared according to any known to the art for the manufacture of pharmaceutical compositions and such compositions may contain one or more agents selected from the group consisting of sweetening agents, flavouring agents, colouring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate: granulating and disintegrating agents for example, corn starch, or alginic acid: binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monosterate or glyceryl distearate may be employed.

Pharmaceutical compositions for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain active materials in admixture with excipients suitable for the manufacture of aqueous suspensions. Such excipients are suspending agents, for example sodium carboxymethylcellulose, methyl cellulose, hydropropylmethylcellulose, sodium alginate, polyvinylpyrrolidone, gum tragacanth and gum acacia: dispersing or wetting agents may be a naturally-occurring phosphatide, for example, lecithin, or condensation products of an alkylene oxide with fatty acids, for example polyoxyethyene stearate, or condensation products of ethylene oxide with long chain aliphatic alcohols, for example hepta-decaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxy-benzoate, one or more colouring agents, one or more flavouring agents or one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavouring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavouring and colouring agents, may also be present.

Pharmaceutical compositions of the invention may also be in the form of oil-in-water emulsions. The oils phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monoleate, and condensation products of the said partial esters with ethylene oxide, for example polyoxyethylene sorbitan monoleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavouring and colouring agents. The pharmaceutical compositions may be in the form of a sterile injectable aqueous or oleaginous suspension. This suspension may be formulation according to known art using those suitable dispersing or wetting agents and suspending agents which have been mentioned above. The sterile injectable preparation may also be sterile injectable solution or suspension in a non-toxic parentally acceptable diluent or solvent, for example as a solution in 1,3-butanediol. Among the acceptable vehicles and solvents that may be employed are water, Ringer's solution, lactated Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The invention also provides a pharmaceutical pack or kit comprising one or more containers filled with one or more of the ingredients, i.e., peptide, carrier, of the pharmaceutical compositions of the invention.

R. Kits: Therapeutic and Diagnostic

The present invention additionally provides for therapeutic kits containing a protein or polypeptide of the invention in pharmaceutical compositions for use in the treatment of animals. The contents of the kit can be lyophilized and the kit can additionally contain a suitable solvent for reconstitution of the lyophilized components. Individual components of the kit would be packaged in separate containers and, associated with such containers, can be a notice in the form prescribed by a governmental agency regulating the manufacture, use or sale of pharmaceuticals or biological products, which notice reflects approval by the agency of manufacture, use or sale for human administration.

The present invention further provides for diagnostic kits for the detection of proteins or nucleic acid molecules of the invention in diagnostic samples. For use in such a kit the protein of the present invention may be labelled with detectable moieties such as radioactive atorns, enzymes, biotin/avidin labels, chromophores, chemiluminescent labels, and the like. The contents of the kit can be lyophilized and the kit can additionally contain a suitable solvent for reconstitution of the lyophilized components. Individual components of the kit would be packaged in separate containers and the kit would further contain instructions on the appropriate method of use of the components.

To gain a better understanding of the invention described herein, the following examples are set forth. It should be understood that these examples are for illustrative purposes only. Therefore, they should not limit the scope of this invention in any way.

### EXAMPLES

### Example 1

### LGL1 in Lung Development

Data from antisense experiments is provided herein showing concentration- and time-dependent inhibition of lung airway branching at rat gestational day 13 by anti-LGL1 antisense oligonucleotides, indicating that lgl1 has an important role in morphogenesis, for example in early branching morphogenesis. Also, LGL1 is maximally expressed in mesenchyme adjacent to the epithelium in the saccular lung, concordant with terminal lung differentiation, demonstrating a potential additional functional role for LGL1 in the maturational processes of late gestation lung development, for example in alveolarization.

In light of its mesenchyme specific localization, and homology to known cell adhesion molecules, it was hypothesized that LGL1 function is required for normal branching morphogenesis in the fetal lung. Using immunohistochemical staining of immediately adjacent serial rat fetal lung tissue sections from fetal days 12-21, the timing and localization of LGL1 mRNA and lgl1 protein were assessed. Anti-LGL1 antisense experiments were used to identify the functional role(s) of lgl1 in lung development.

Materials and Methods

Drugs and chemicals were obtained from the following sources: culture media (minimal essential medium; MEM), nylon (Hybond N) membranes from Amersham.; penicillin, streptomycin, urea, agarose, TRIZOL, ethidium bromide, random hexanucleotide primers, Taq polymerase, restriction endonucleases from Gibco/BRL Life Technologies, Burlington, Ont.; PCR pimers, Sheldon Biotechnology, Montreal, QC; Sequenase from Amersham; deoxynucleotides and RNA Guard RNase inhibitor from Pharmacia Biotech Inc, Baie d'Urfé, PQ; ³²Pα-dCTP from Dupont Canada, Mississauga, Ont.

RT-PCR Analysis

Total (nuclear and cytoplasmic) RNA was prepared from fetal rat lung tissue using the TRIzol reagent, according to manufacturer's instructions. Briefly, whole rat lungs were homogenized in TRIzol reagent and RNA extracted with chloroform. RNA was ethanol precipitated, collected by centrifugation, lyophilized, and dissolved in RNase free water. Three μg of total RNA from explants was used for reverse trancription-polymerase chain reaction (RT-PCR). RT-PCR reactions were carried out using 27 cycles of PCR for LGL1 and 25 cycles for β-actin, to control for loading and integrity of samples. Each cycle consisted of denaturation (45 seconds at 94° C.), annealing (45 seconds at 55° C.), and extension (4 minutes at 72° C.). Synthetic oligodeoxynucleotide pairs were designed corresponding to conserved sequences of rat LGL1, yielding a 472-bp product and β-actin, yielding a 515-bp product. The following sets of PCR primers were used: LGL1 forward primer, 5′-ATG CTG CAC AAC AAG GCT GCG-3′ (SEQ ID NO. 5); reverse primer, 5′-GCT CTG AGT GTC CGT CCA GCT-3′ (SEQ ID NO. 6); β-actin forward primer, 5′-GTG GGC CGC TCT AGG CAC CAA-3′ (SEQ ID NO. 7); β-actin reverse primer, 5′-CTC TTT GAT GTC ACG CAG CAT TTC-3′ (SEQ ID NO. 8). DNA contamination was excluded by performing PCR of each sample without first transcribing mRNA with MuLV reverse transcriptase. RT-PCR products were separated on 1.5 % (w/v) agarose gels and visualized by ethidium bromide staining.

In Situ Hybridization

Non-radioactive in situ hybridization was performed as described by Moorman et al. (J Histochem. Cytochem. (2001) 49: 1-8), using a 1.4 kb LGL1 digoxigenin-labled RNA probe corresponding to nucleotides 1154-2620 of SEQ ID NO: 1 (see also Kaplan et al (1999) Am. J. Physiol. 276 (Lung Cell. Mol. Physiol 20): L1027). Rat LGL1 cDNA flanked by Kpn I and Sma I sites, subcloned into pBluescript KS, was used as a template for in vitro transcription. Riboprobes were generated after linearization and in vitro transcription by T3 or T7 polymerase labeled in the presence of dig-UTP. Briefly, tissue sections were deparaffinized, rehydrated, and washed in PBS. Pretreatnent included proteinase K digestion (20 μg/ml, 15 minute at 37° C.), termination in glycine (0.2% in PBS, 15 minute, room temperature), and postfixation in 4% paraformaldehyde/0.2% gluteraldehyde (15 minute, room temperature). Sections were then washed in PBS, and prehybridized for 1 hour at 70° C. Riboprobes were added to freshly prepared hybridization solution (50% formamide, 5×SSC pH 4.5, 1% Boebringer block, 1 mg/ml yeast tRNA, 5 mM EDTA, 0.1% Tween-20, and 0.1% CHAPS) at a concentration of 1.5 ng/μl. Following denaturation at 100° C., the probe was incubated with tissue sections at 70° C. for 18 hours. Tissues were then washed with 50% formamide and 2×SSC at 65° C. The dig nucleic acid detection kit (Boehringer Mannheim) was used for immunological detection of the hybridized probe. Tissues were then dehydrated and mounted with xylene. Images were captured using a Leica digital imaging system. Adjacent serial sections were kept for immunostaining with anti-vimentin and anti α-actin antibodies, in order to identify the positively stained LGL1 cells.

Fetal Rat Lung Explant Culture

Time mated Wistar rats (Charles River, St. Constant, Quebec) were killed by diethylether excess on days 12 or 13 of gestation. Embryos were aseptically removed from uterine decidua and fetal lung rudiments were isolated from the embryos by microdissection. Lung rudiments were cultured in serum-free, chemically defined medium (DMEM/F12 (1:1 mix, Gibco) supplemented with 100 units/ml penicillin, 100 ug/ml streptomycin, and 0.25 mg/ml ascorbic acid). Four to five lungs, gestational day 13, were placed on each porous membrane insert (0.8 μm pore size, 25 mm diameter, Nucleopore, Whatman, N.Y.) in each of six separate wells of a culture plate (Costar, Coming N.Y.) containing 800 μl of culture medium. Explants were incubated as floating cultures with an air-liquid interface for 48 hours at 37° C. in a humidified atmosphere of 5% CO2 in 3% O2. Lung explants were treated to either 30 or 40 μM of antisense, sense, or scrambled oligodeoxynucleotides (see below). Untreated explants (incubated in serum free media alone) served as additional controls.

LGL1 Antisense Oligodeoxynucleotides

Phosphorothioate oligodeoxynucleotides (ODNs) of 18 base pairs, targeted against sequences adjacent to the ATG initiation codon of LGL1 mRNA were synthesized (DNA synthesis facility, HSC, Toronto, ON.). The antisense (As1) LGL1 oligodeoxynucleotide was: 5′-GTT GTG CAG CAT GAG GAT-3′ (SEQ ID NO. 9). The corresponding control sense (S1) was 5′-ATC CTC ATG CTG CAC AAC-3′ (SEQ NO. 10). The scrambled sequence (Sc1) had the same nucleotides as As1, but with a randomly scrambled sequence: 5′- GTA CTT GAC GCT TGA GAA-3′ (SEQ ID NO. 11). ODN sequences were modified by phosphothiolation to improve stability against nuclease degradation in culture. The ODNs were purified by HPLC, dissolved in double distilled water and quantified by UV spectrometry at a wavelength of 260 nm.

Oligodeoxynucleotides (ODNs) can be used to specifically inhibit the translation of gene products in the fetal rat lung. Antisense (As) ODNs targeted to sequences adjacent to initiation codons (ATG) are very efficient in inhibiting translation, due to their ability to interfere with ribosome binding.

Quantification of Branching Morphogenesis Lung explants (day 12 or 13) were monitored daily by phase contrast microscopy. To quantitatively assess branching morphogenesis, the terminal airway buds were counted after 0, 24, 48 and 72 hours of incubation. Representative explants were photographed after 48-72 hours in culture.

Western Blot Analysis

The LGL1 protein content was determined in day 13 fetal lung explants cultured for 48 hours in the presence of ODNs, and in rat lung rudiments from gestational ages 13-21. Briefly, lungs were homogenized in RIPA buffer containing triton X-100, sodium deoxycholate, SDS, NaCl, Tris pH 8.0, and protease inhibitors. The total protein concentration was determined according to Bradford ((1976) Anal. Biochem. 72: 248-254). Fifty μg of protein diluted with sample buffers was loaded in each well on a 10% (w/v) SDS polyacrylamide gel. Transfer efficiency was determined by Ponceau staining. A rabbit polyclonal lgl1 antibody raised against a synthetic peptide corresponding to amino acid residues 417-434 of rat lgl1 was generated (Medicorp, Montreal, Canada). Immunodetection was done according to standard protocol with few modifications. Briefly, non-specific binding was blocked by incubation with 2% normal goat serum (NGS) in TBS-T at room temperature for 90 minutes. The membrane was then incubated with rabbit anti lgl1 at room temperature for 90 minutes, washed 4 times with TBS-T and then incubated with hrp-conjugated goat anti rabbit IgG (1:10000) in TBS-T containing 2% NGS. After TBS-T washes, blots were developed with an ECL kit. The films were quantified by laser densitometry.

The lgl1 antibody binds to a 45 kD band, consistent with the deduced size of the lgl1 protein. This band was substantially eliminated by pre-absorption of the antibody with a 5-fold excess of the peptide used in generation of the antibody.

Immunohistochemistry

Rat lung tissue from gestational ages 13, 16, 19 and 20 were fixed over night at 4° C. in Bouin's fixative, embedded in paraffin and cut into 5 μm sections. Tissue sections were immunostained according to Naish et al. with few modifications. In short, antigen was retrieved by incubation in boiling 0.M sodium citrate pH6 for 10 minutes. Endogenous peroxidase activity was quenched with 1.5-% (v/v) hydrogen peroxide in methanol for 45 minutes. Non-specific binding sites were blocked using 5% (v/v) NGS and 1% (w/v) BSA in PBS for 1 hour at room temperature. Preliminary experiments determined optimal antibody concentrations. Rabbit antibody against lgl1 was used at 1:400 dilution and mouse antibody against vimentin and α-actin were used at 1:250 and 1:1000 respectively. Sections were incubated with primary antibodies overnight at 4° C., washed in PBS and then incubated with secondary antibody, either biotinylated anti rat IgG at 1:500 or biotinylated anti mouse IgG at 1:400, for 2 hours at room temperature. After washing with PBS, slides were incubated with ABC for 1 hour at room temperature and developed using DAB substrate (Vector laboratories). Tissues were then counterstained with hematoxylin and prepared for viewing.

Statistical Analysis

All data are presented as mean values±S.E.M. Statistical significance was determined by two-way Analysis of Variance (ANOVA). Pair-wise group comparisons were then assessed using Student-Neuman-Keuls test. Significance was defined as p<0.05.

Results

LGL1 mRNA in the Developing Lung

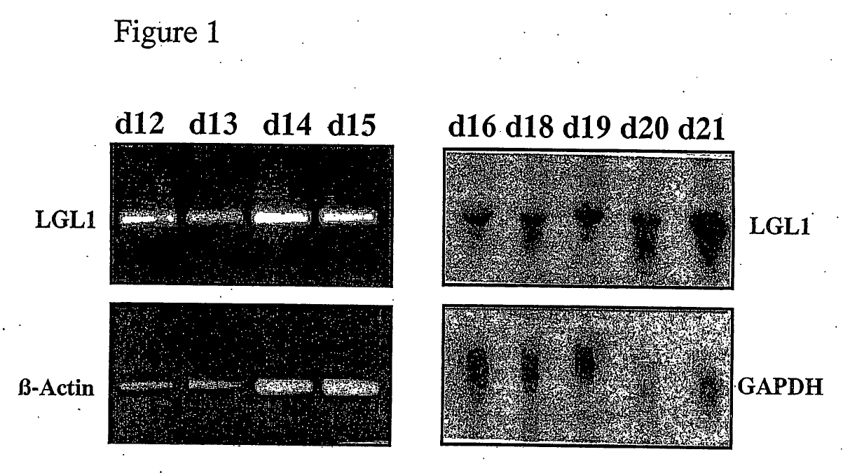

The developmental expression of lgl1 during lung development was examined using RT-PCR (days 12-15) and northern analysis (days 16-21; FIG. 1). LGL1 mRNA was detected as early as d12, corresponding to the onset of epithelial branching morphogenesis; with maximal expression at d21 of gestation.

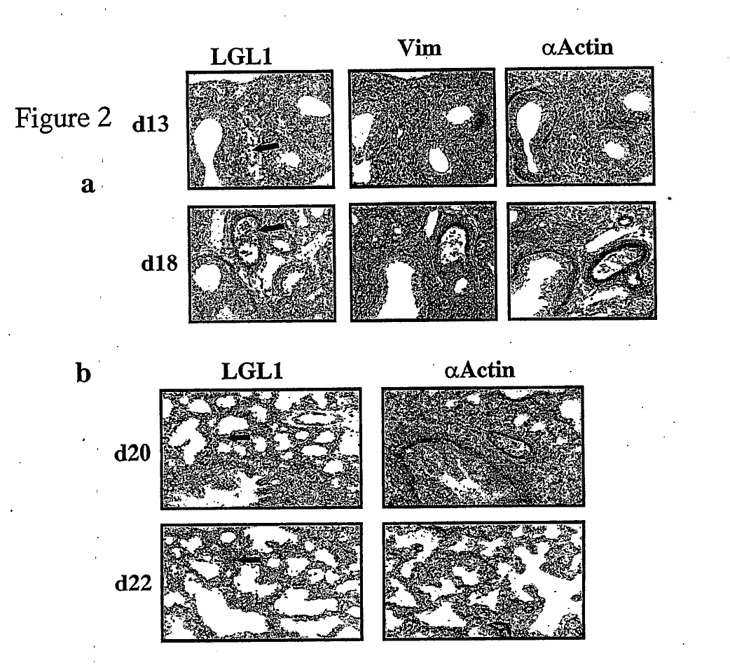

By in situ hybridization (ISH), LGL1 mRNA was localized exclusively in the mesenchyme of day 13 fetal rat lung, with a homogeneous distribution (FIG. 2a), but with no detectable expression in the epithelium. This was confirmed by immunostaining of immediately adjacent sections with anti vimentin antibodies, a mesenchymal marker (FIG. 2b). This mesenchyme specific pattern of expression is still conserved later in gestation. In the cannicular (day 18) stage of rat lung development, LGL1 mRNA is still found throughout the mesenchyme, but there is also an increasing population of positively stained LGL1 cells that localize in smooth muscle cells surrounding large and small blood vessels, and large airways (FIG. 2c). This was confirmed by imnmunostaining of immediately adjacent sections with antibody against α-actin, a smooth muscle cell marker (FIG. 2e). During the saccular stage of rat lung development (days 20-22), LGL1 mRNA spreads throughout the mesenchyme, with patchy expression in the smooth muscle cells of blood vessels and large airways (FIG. 2f).

Lgl1 Protein in the Developing Lung

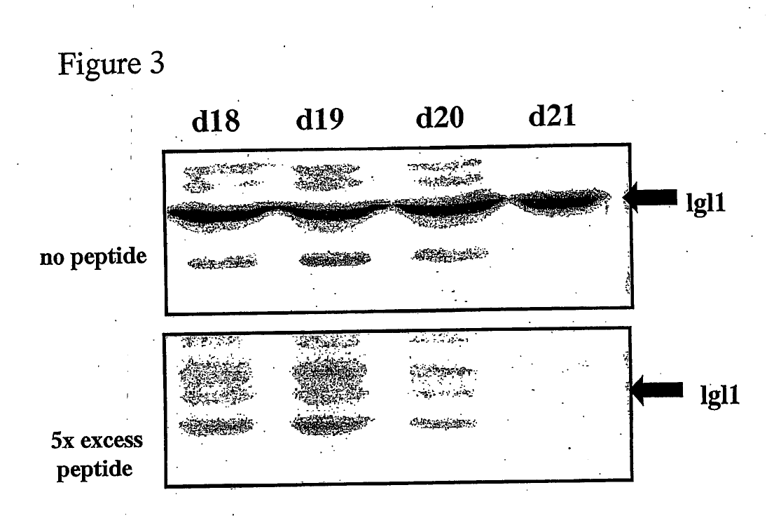

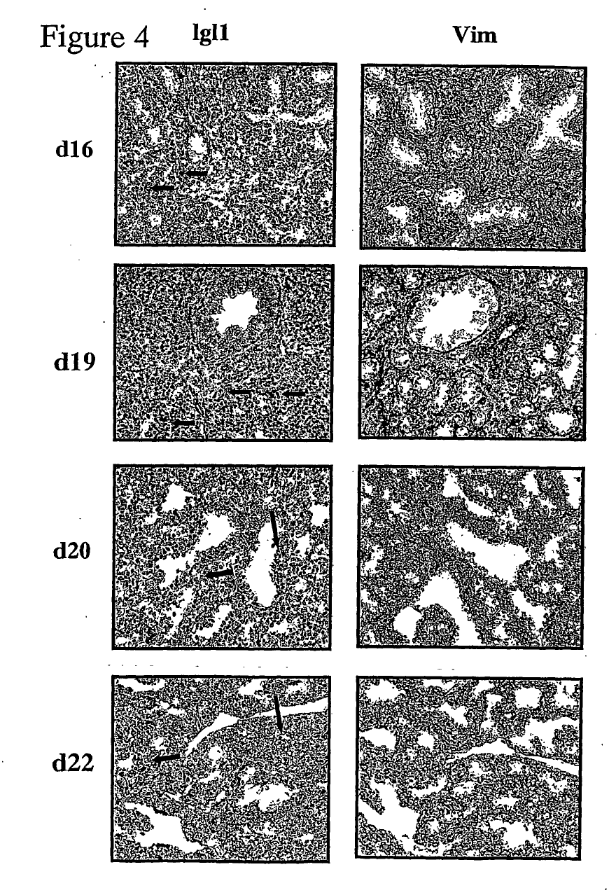

Lgl1 protein is expressed in fetal lung as early as day 18 by Western blotting (FIG. 3a), and on day 13 by IHC (FIG. 4a), the earliest stages studied by either method. Both methods showed that lgl1 protein levels in the lung gradually increase, reaching a maximum on day 21.

The spatial localization of lgl1 protein reflected that of its mRNA. Lgl1 protein was localized exclusively to mesenchymal cells at all gestational ages tested, but the lgl1 positively stained cell populations were not spread homogeneously throughout the mesenchyme. Rather, “patchy” clumps of positively stained cells, localized exclusively in the mesenchyme, were observed throughout gestation (FIG. 4a-d). Earlier in gestation (day 16), positively stained clumps of cells are proximal to small airways and large vessels (FIG. 4b). Later in gestation (day 21), positively stained cells are still proximal to small airways (FIG. 4d).

Disruption of Endogenous LGL1 Activity Results in Impaired Branching Morphogenesis

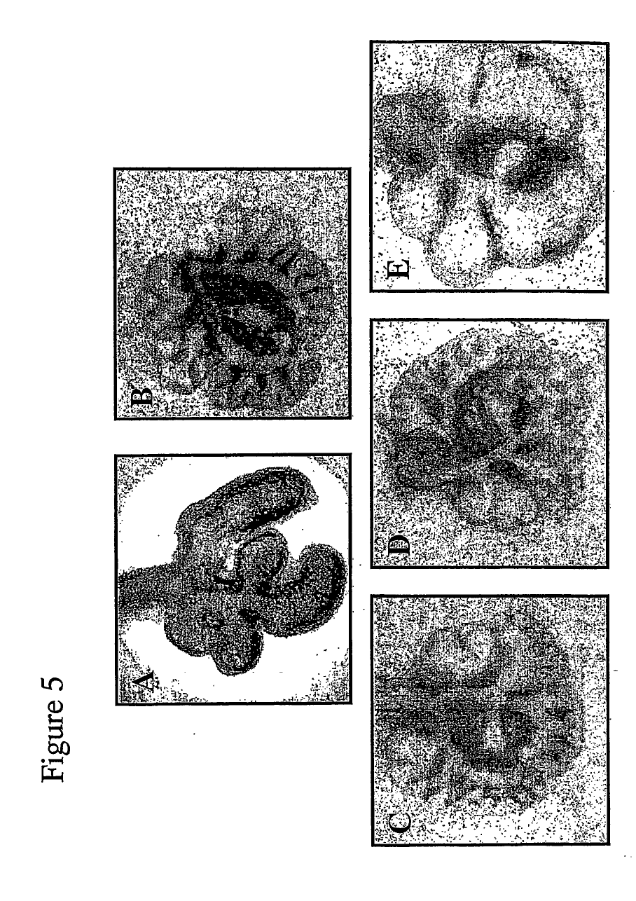

To assess the biological function of LGL1 in lung development, embryonic lungs were cultured in the presence of antisense ODNs specific for LGL1. Rat lung rudiments isolated at d13 have right and left mainstem bronchi (FIG. 5a) with a few rudimentary branches. After two days in culture, the lung rudiments are increased in size. Subsequent dichotomous branching of the bronchi has formed a respiratory tree, but it is reduced in comparison to the in vivo situation (FIG. 5b). Day 13 rudiments were explanted in culture in the presence of two non-toxic doses (30 and 40 μM) of LGL1 antisense ODNs. After 48 hours in culture, there was no difference in morphology between controls exposed to medium only and explants treated to sense or scrambled ODNs, while explants treated to antisense ODNs showed significant dilation and reduction in number of terminal airway buds (FIG. 5e).

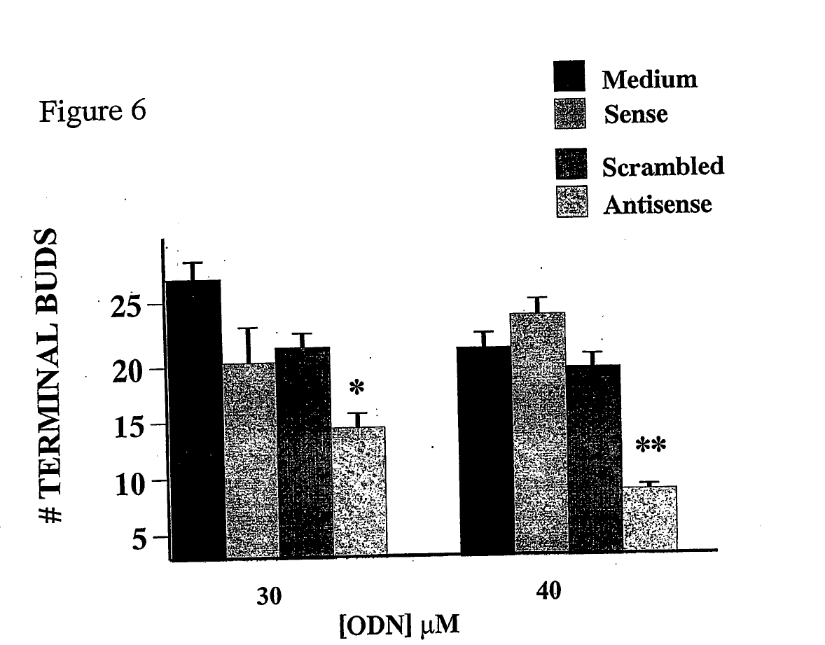

The amount of branching was quantified by counting the number of terminal airway buds at the end of 48 hours. Branching was decreased by 47% and 57% in explants exposed to 30 and 40 μM antisense ODNs (FIG. 6). Addition of the same concentration of either sense or scrambled LGL1 ODNs had no effect on branching compared to medium controls (FIG. 6). This effect of antisense LGL1 ODNs was more pronounced in explants exposed to the higher concentration of antisense ODNs. These results support the conclusion that inhibition of terminal branching by LGL1 antisense ODNs was specific and dose dependent.

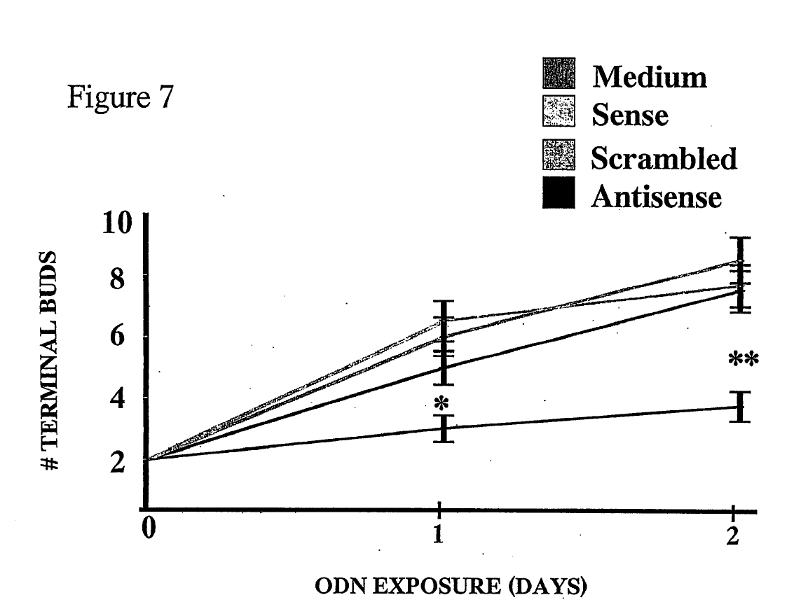

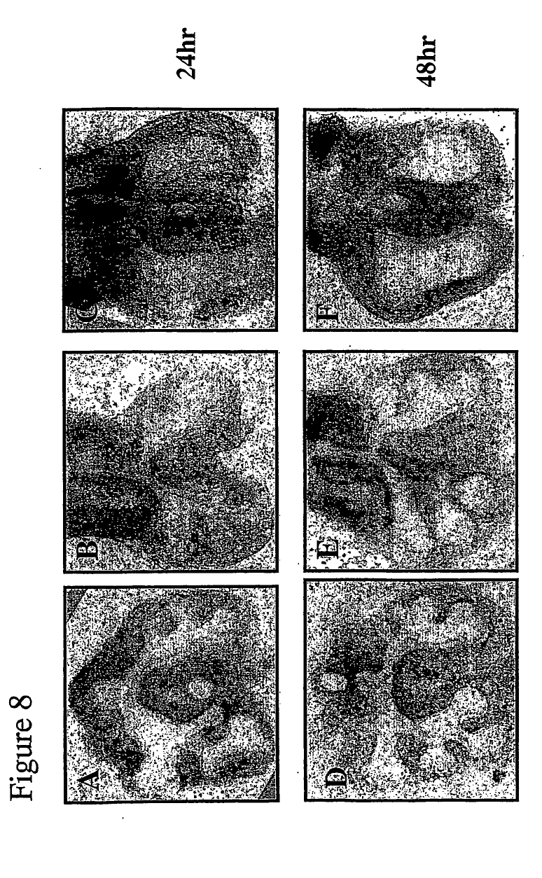

This inhibitory effect of antisense LGL1 ODNs on branching morphogenesis, is more pronounced when lung rudiments from day 12 are cultured for 48 hours (FIG. 7). At day 12 in the rat, the tracheal epithelium is just starting to bud and form mainstem bronchi. The airways of day 12 antisense treated (40 μM) explants do not branch. Instead the terminal airway buds simply dilate over time, while in all control treated explants, progressive branching is observed (FIG. 8a-f).

Effect of Antisense LGL1 ODNs Treatment on LGL1 mRNA and lgl1 Protein Expression in Explanted Rat Lungs

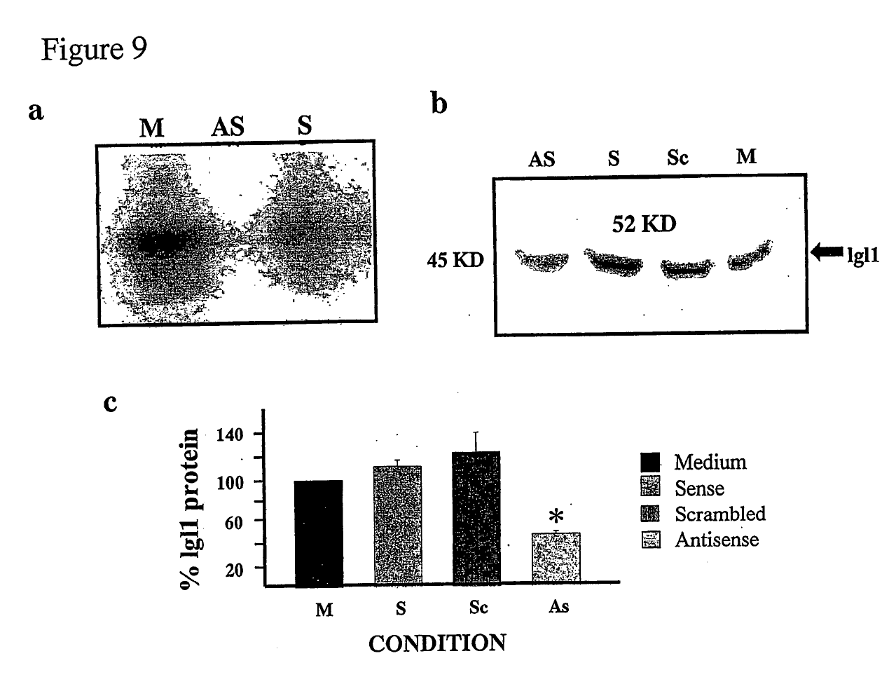

The specificity of the reduction in LGL1 mRNA levels in response to treatment with anti-LGL1 antisense ODN was confirmed by the lack of response to sense ODN, and by the lack of alteration in β-actin mRNA levels (FIG. 9a). To determine the degree of inhibition of endogenous lgl1 protein production by antisense LGL1 ODNs, the lgl1 protein content of day 13 explants cultured for 48 hours was assessed by western blotting and quantified by densitometry. The treatment with LGL1 antisense ODNs significantly reduced the total amount of lgl protein in cultured explants by 51% (FIG. 9b). No reduction of lgl1 protein content was observed in any control explants (FIG. 9b).

These antisense data show that reduction of the normal levels of the LGL1 gene product is associated with a profound reduction in lung formation. While airway growth continues there was a marked reduction or even cessation in the normal rapid formation of new airway branches. Existing evidence shows that factors involved in supporting airway branching morphogenesis are also involved in alveolarization. The demonstrated role of lgl1 in airway branching and the timing of its maximal expression indicates that lgl1 will have a significant role in alveolarization.

A concentration- and time-dependent anti-LGL1 inhibition of lung airway branching has been demonstrated at gestational day 13 in the rat, indicating that lgl1 has an important role in early branching morphogenesis. Immunohistochemical staining of immediately adjacent serial tissue sections has revealed LGL1 mRNA and protein diffusely throughout fetal rat lungs of gestational days 13-16. Maximal LGL1 expression occurs late in gestation (days 20-21), when the branching of the conducting airways is complete, and the formation of alveolar (gas-exchange) units is beginning. During this developmental stage, the mRNA and protein become progressively restricted to mesenchymal cells, positive for smooth muscle actin, which are in close proximity to airway epithelial cells. Interestingly, these same actin-positive cells have been reported to regulate the formation of new alveolar units. Taken together with the above data, this indicates that lgl1 plays a role in the regulation of alveolarization.

### Example 2

### LGL1 Sequence

A nucleotide sequence is provided in SEQ ID NO: 1 for rat late gestation lung 1 gene (LGL1) and in SEQ ID NO: 3 for human LGL1 gene. The instant nucleotide sequence for fetal rat lung LGL1 (3054 bp), and the associated deduced lgl1 protein (497 amino acids), are presented herein (SEQ ID NO: 1 and 2). A partial sequence of the human LGL1 nucleic acid molecule (82 nucleotides) and lgl1 protein (27 amino acids) has been determined and are provided as SEQ ID NO: 12 and 13. A full-length nucleotide sequence of human LGL1 was cloned using standard PCR technique from human fetal lung cells in culture, and is provided as SEQ ID NO: 3 (2896 nucleotides) and the deduced amino acid sequence is provided as SEQ ID NO: 4 (497 amino acids). The full length human sequence of SEQ ID NOs: 3 and 4 show 100% identity with the fragment of SEQ ID NOs: 12 and 13, respectively, which encodes amino acids 61-87 (nucleotides 212-292) of the full-length sequence.

The rat LGL1 sequence incorporates changes to the previously reported partial sequence. of LGL1 and yields a deduced protein having an 5′ region that is extended to yield a 497 amino acid protein, compared to the 437 amino acid polypeptide reported by Trexler et al. (Eur. J. Biochem. (2000) 267: 5751-5757), which in turn was a 3′-extension of the 188 amino acid sequence initially reported by Kaplan et al. ((1999) Am J. Physiol. 276 (Lung Cell. Mol. Physiol. 20) L1027-L1036; GenBank accession number AF109674). In addition to having a 5′-extension, the fetal rat lung LGL1 nucleotide sequence disclosed as SEQ ID NO: 1 differs from the partial sequence by Trexier et al. (Eur. J. Biochem. (2000) 267:5751-5757) as follows (positions refer to the nucleotide sequence of SEQ ID NO: 1):
1. position 129: T replaces C
2. position 171: C replaces T
3. position 206: G replaces A
4. position 256: insertion of G (SEQ ID NO: 1 has 2 “G” instead of 1 “G”)
5. position 1481: removal of A (SEQ ID NO: 1 has 2 “A” instead of 3 “A”)
6. position 1551: insertion of C (SEQ ID NO: 1 has 2 “C” instead of 1 “C”)
7. position 1577: T replaces G
8. position 1691: removal of C (SEQ ID NO: 1 has 2 “C” instead of 3 “C”)

The nucleotide sequence of Trexler et al. differed from the initially described sequence of Kaplan et al. in having a single base (G) insertion following nucleotide 819 which generated a frame shift following codon 185, and thus obviated the stop codon initially described following amino acid residue 188. The same single nucleotide insertion was confirmed experimentally by the inventors of the instant application.

The lgl1 protein is expressed as a precursor peptide containing a hydrophobic signal sequence that is 24 amino acid residues long with the cleavage site is at amino acid 22. Thus the mature protein (rat and human) starts with codon 22 and extends to codon 497, yielding a protein of 476 amino acid residues. The lgl1 predicted secondary structure includes a very hydrophobic region at amino acids 1-26 (corresponding to the signal peptide), and two N-linked glycosylation sites at the aspartate residues at codons 27 and 199.

At amino acids 54-208, the predicted protein contains the SCP-like extracellular protein signature classified to a family of evolutionary related proteins with extracellular domains, thought to have a role in defense, e.g. as part of the immune system. Two CRISP signature motifs are found at about amino acids 160-169 and about amino acids 195-206, indicating that lgl1 is a member of the CRISP family of cysteine-rich, androgen-regulated secreted proteins. The second signature contains a cysteine residue that is involved in a disulfide bridge in members of the family {Kaplan et al. (1999) supra}. The lgl1 amino acid sequence partially aligns with P25TI (a peptide with weak trypsin inhibitor activity) but little homology is detected at the level of the nucleotide sequence. In contrast, the rat and human sequences provided in SEQ ID NOs: 1-4 show at least 98% identity over the entire nucleotide sequences and the encoded polypeptides.

The LCCL1 and LCCL2 domains reported by Trexler et al. are located at about amino acid 284-379 and about amino acids 385-490 (positions refer to the nucleotide sequences of both SEQ ID NOs: 1 and 3).

A Blast of SEQ ID NO: 4 against the GenBank protein database indicated that the full-length protein has some homology/similarity with the CocoaCrisp, a protein involved in brain septation (human sequence GI 13899303/NP_{—}113649.1, gallus gallus sequence GI 13241978/AAK16497.1 and mus musculus sequence GI 13878237/NP_{—}113579.1).

### Example 3

### LGL1 in Disease

The involvement of LGL1 in both the morphogenetic and maturation aspects of lung development is of particular interest in light of observed pathologies of lung disease marked by abnormal lung morphogenesis or alveolarization or an arrest of lung development in late gestation.

Lgl1 Expression in Disease States

Clinical and laboratory (animal model) sources of lung tissue are used to establish that lgl1 expression is altered in various disorders of branching morphogenesis or disease states associated with abnormal alveolarization, whether of primary or secondary origin, including emphysema, COPD, CDH, BPD and New BPD. Fixed, frozen and fresh tissue sections from human lungs with a variety of disease states, including emphysema and COPD, and various animal models may be used, for example canine emphysema, baboon or rat BPD (see below), and ovine or rabbit CDH. For clinical emphysema, COPD, CDH and BPD, sections may be sub-classified according to severity of pathology, and gender. Emphysema samples may also be classified by pathological subtype (pan-acinar, centriacinar), Pi type (alphal-anti-trypsin genotype) and history of exposure to cigarette smoke.

The amount of lgl1 protein and LGL1 mRNA is quantified (e.g. per gram of tissue) using standard techniques (Western and Northern blot analyses, respectively) in human and animal tissues. Where expression levels of mRNA are too low for detection by Northern Blot, semi-quantitative estimations of relative levels are made using RT-PCR methods (e.g. see Example 1). The relative amount and spatial distribution of expression within the lung is determined using immunohistochemical methods and in situ hybridization, for example with scoring of relative intensities of expression in mesenchymal cells (fibroblasts and actin-positive smooth muscle cells) by a blinded observer {Sweezey, N. et al. Am. J. Physiol. (Lung Cell Mol. Physiol.) (1998) 275: L103-L109}. Disease-free sections from lung resections for cancer are used as controls.

LGL1 Alleles Associated with Increased for Lung Disease States

Recent studies have shown that specific polymorphisms within genes can be associated with increased risk for disease states {Frosst, P et al. Nature Genet (1995) 10:111-113; Ma, J. et al. Cancer Res. (1997) 57:1098-1102}. Given the massive burden of diseases like emphysema, and its association with LGL1 expression, there may be an altered risk for various human pathological conditions associated with the presence of specific LGL1 alleles. Single nucleotide polymorphisms (SNPs) will be identified in the LGL1 gene using standard techniques known in the art (for example, single stranded conformational polymorphisms, direct sequencing) {see, for example, Rozen, R. in Carmel, R. and D. W. Jacobsen, eds. Homocysteine in Health and Disease. Cambridge, Cambridge University Press. 2001, 259-269}. DNA will be isolated (from each of the disease sub-classifications above, and from control tissue) from banked tissue sections, and from clinic patients, including CDH and BPD patients.

Methods of Identifying Modulators of lgl1 Biological Activities

Ex vivo assays on explanted lung sections or on established or primary cell lines may be used to test the ability of compounds of the invention or therapeutic agents to modulate lgl1 biological activities such as branching morphogenesis and/or alveolarization during normal development and in various pathological conditions of the mammalian lung. The lung sections may be derived from diseased and normal human lung samples or from animal models of disease. Morphometric approaches can quantitate the degree of alveolarization and/or branching morphogenesis, estimating among other things the number of alveolar units or branches in a given three-dimensional volume of tissue. These methods use systematic sampling and counting of structures in histological sections.

Explant culture assay: Sources of sections of fresh surgical specimens of excised human lung include emphysema patients undergoing volume reduction resections or transplant, with control tissue samples coming from the normal margins of lungs resected for cancer. Where available, sections from patients with BPD or CDH or other lung disorders, such as conditions involving pulmonary hypoplasia can also be used. Fresh explant lung cultures from late gestation and early post-natal rats, as well as from a rat model of BPD, an ovine model of CDH and a canine model of emphysema are also suitable sources of lung tissue. Serial tissue sections of standard dimensions (<0.5 mm in thickness) will be maintained in explant culture in defined serum free-medium as is well-known in the art (see Example 1).

Sections are assigned to a series of treatment groups, based on a range of previously optimized concentrations, incubation periods and delivery approaches. A separate series of experiments may be conducted to test for differences in efficacy between various candidate compounds.

To monitor branching morphogenesis, increasing concentrations of the candidate compound are incubated with explants of fetal rat lung (fetal day 12 or 13), and the explants observed and terminal end buds counted after 0, 24, 48 and 72 hours of incubation as a marker of airway branching. To assess alveolarization, alveoli per high-powered field under light microscopy are quantified in treated sections and compared to control samples. Data is segregated by age, gender and smoking history where numbers permit meaningful sub-group analysis. Differences between treatment groups may be evaluated, for example using ANOVA, followed by Neumann-Student-Keuls test for pair-wise inter group comparisons.

The effect upon alveolarization may also be assessed in traditional cell culture. Suitable cells for test treatment include stem cells or progenitor cells isolated from lung tissue, for example either rat or human lung tissue, although lung tissue from any animal may be used. In this embodiment, the tissue would be cultured in media containing the candidate compounds, or example the polypeptides of the present invention or agents that modulate lgl1 expression or activity. Any conventional media that supports growth can be used, for example DMEM. The media may be supplemented with other soluble and insoluble factors to promote the alveolarization. Suitable soluble factors include, for example, fetal calf serum, prostaglandin, hydrocortisone, triiodothyronine, selenium, insulin, insulin growth factor I, Wnt-1, Wnt-4, fibroblastic growth factor, hepatocyte growth factor and combinations thereof. Suitable insoluble factors include, for example, Type I collagen, Type IV collagen, laminin, proteoglycans, fibronectin, pronectin-F, pronectin-L, recombinant adhesion molecule and combinations thereof. Primary lung cell culture techniques are described, for example, by Kaplan et al., ((1999) Am J Physiol 276 (Lung Cell. Mol. Physiol 20): L1027-L1036).

Alveolar Formation Studies:

Isolation of Type 2 Alveolar Cells (AEC2 cells): Type 2 alveolar cells may be isolated from 250 g Sprague Dawley rats according to the method of Rannels (Rannels and Rannels, 1988). Briefly, an initial cardiac perfusion with 0.9% saline is followed by instillation of the airways with 0.1% elastase (e.g. Calbiochem Corp., La Jolla, Calif.) in Joklik's modified minimal medium (JMEM) containing 0.05% BaSO.sub.4. Elastase is inactivated by instillation of JMEM containing soybean trypsin inhibitor (0.08%; e.g. Sigma Chemical Co., St. Louis, Mo.), DNase (0.08%. e.g. Sigma Chemical Co.), and 50% newborn calf serum. Lung tissue is minced, vortexed, and filtered through 160 μm nylon mesh (e.g. Tetko, Elmsford, N.Y.). Released cells are centrifuged for 10 min at 500 g, resuspended in JMEM containing 0.08% DNAse, and layered on a Percoll discontinuous density gradient. After centrifugation for 20 min (4° C.), cells are collected at the 1.04/1.08 interface, washed in JMEM, brought up in DME containing 10% fetal bovine serum (FBS). Cells are then incubated for 30 min (37° C.) in 75 cm² tissue culture flasks to eliminate contaminating macrophage cells which rapidly adhere to the plastic surface.

The resultant type 2 alveolar cell preparations may be 95% viable as determined by trypan blue exclusion, and over 90% pure as assessed by the presence of lamellar bodies visible with Hoffman optics at a magnification of 40 and by tannic acid staining. Analysis of total cellular DNA/well on each day of an experiment is performed in triplicate on trypsin/disease released cells using a DNA fluorometry assay {Labarca and Paigen, (1980) Biochem. 25:312-318}. No contaminating DNA may be detected in wells containing substrate alone, which after dissolution with dispase and trypsin did not contribute to the cell pellet.

Assessment of Alveolar Formation: To study alveolar formation, freshly isolated type 2 alveolar cells may be plated in 96-well plates at 20×10³ cells/well on 500 μg/well (1.8 mg/cm²) of gelled substrate of Collagen I, for example basement membrane substrate (BMS) prepared according to Paulsson et al. {(1987) Eur. J. Biochem. 166:11-19}. In one approach, cells are cultured over 5 d with one media change performed on day three. Alveolar formation is analyzed at 24 h intervals over 5 d, for example using photographic negatives (4× original magnification of central portion of each well) of triplicate wells. Images from negatives are transferred via video camera to an Image I imaging system (Universal Imaging Corp., West Chester, Pa.) and viewed on a colour video monitor. The area of cellular structures may then be determined and expressed as the mean±standard deviation. In some cases two size categories are distinguishable: (a) single cells (200-300 μm²) and (b) model alveoli (20×10³ μm² or greater) with data expressed as percent of area occupied by each category.

Alveoli-like structures typically become apparent 3-5 d after plating dispersed type 2 alveolar cells on gelled substrate. These structures have a central lumen lined by a cuboidal epithelium of lamellar body containing type 2 alveolar cells. Type 2 cells of appropriate polarity will bear a striking resemblance to their in vivo fetal counterparts prior to the appearance of type 1 cells {Adamson and Boden, 1975}.

Screening for compounds capable of modulating alveolar formation: To determine whether in vitro alveolar promoting or inhibiting activity resides in candidate compounds, the type 2 alveolar cells are pre-incubated with increasing micromolar amounts of the compound prior to plating on substrate. Alternatively, promotion or inhibition of alveolarization due to the absence of naturally occurring polypeptides may be assessed. To test this directly, a candidate compound is pre-incubated in increasing micromolar amounts with freshly isolated dispersed type 2 alveolar cells prior to plating on substrate. Relative to control cultures pre-incubated with vehicle alone, changes in the qualitative and quantitative endpoints of alveolar formation, as described above, demonstrate the in vitro alveolarization potential of the candidate compound. Cell viability is monitored as a marker of non-specific cytotoxicity of the candidate molecule.

To define further the critical region(s) in a biologically active molecule, where the candidate compound is a polypeptide, it may be trypsin digested, generating the smaller peptides. Each smaller peptide is then HPLC purified, sequenced, and pre-incubated with type 2 alveolar cells at increasing micromolar concentrations to determine it's effect on alveolarization.

Type 2 alveolar cells can also be examined as sectioned cultures, in which case they are plated on gelled substrate supported by Millicell™ 0.4 μm filter inserts. After 5 d, with or without exposure to a candidate compound, cultures are fixed for 1 h with 2% formaldehyde/2% glutaraldehyde in 0.05M sodium phosphate buffer, pH 6.8, and washed. Filters are cut out, treated for 1 h with 1% osmium tetroxide, acetone dehydrated, and embedded in resin. Semi-thin or thin sections are then cut, stained and examined in the light or electron microscope, respectively. See, for example, U.S. Pat. No. 5,696,229 issued to Laurie et al. on Dec. 9, 1997 and is incorporated herein by reference.

In alveolar inhibition studies, antibodies (50 μg/well) against a candidate compound are incubated with gelled substrate in wells of 96-well plates for 60 min at 37° C. Unbound antibody is removed by three DME washes (200 μl/well) and then freshly isolated cells are added. Dose-response assays are performed on all antibodies. Candidate compounds are pre-incubated with freshly isolated type 2 alveolar cells in suspension for 30 min at 37° C. with gentle agitation every 5 min; cells together with the candidate compound are then plated on the antibody treated substrate. Cell viability in the presence of each antibody, candidate compound is assessed using the MTT (3-(4,5-dimethylthiazol-2-yl)-2,5-diphenyl tetrazolium bromide) assay known in the art.

Lgl1 Effect on Lung in Mammalian Models of Disease, In Vivo

In vivo experiments are used to establish the pre-clinical safety and efficacy of drug candidates. Effects on branching morphogenesis (in several tissues, see Example 4) and alveolarization during normal development are assessed by treating (non-human) pregnant mothers before and/or during the gestational stages of branching morphogenesis (for various tissues) and alveolarization. For example, pregnant mice might be treated before, on and/or after Embryonic Day 9 when the two primary buds arise on the embryonic foregut. Effects on branching morphogenesis (in several tissues) and alveolarization in diseased tissue are assessed using similar protocols, except the treated subjects are animal models of disease as outlined above. Actual dosing amounts and schedules are determined based on the results obtained in explant cultures, and the stage of gestation or of disease progression in the animal model.

### Example 4

### LGL1 in Non-Lung Tissues

LGL1 is also expressed in fetal and adult tissues other than the lung, demonstrating a role for LGL1 in development in these tissues, for example in kidney morphogenesis. Methods useful to test the activity of candidate molecules of the LGL1 family are known in the art. A sample description of methods used to test the activity of candidate molecules on polycystic kidney disease is provided.

In Vitro Polycystic Kidney Disease (PKD) Evaluation: Metanephric Organ Culture

To demonstrate that aberrant morphogenetic effects of the LGL1 expressed in the developing and adult kidney contribute to cyst formation in polycystic kidney disease (PKD), the ability of the compounds of this invention to treat or inhibit polycystic kidney disease can be determined in vitro and in vivo in mice. The standard pharmacological test procedures described below emulate polycystic kidney disease in humans. {See P. Frost, U.S. Pat. No. 5,929,080 issued in 1999}.

An in vitro standard pharmacological test procedure may be used to measure the ability of a candidate compound to promote or inhibit the formation of tubular cysts (cystic index) in fetal mouse metanephros cell culture. The procedure using serum-free culture of fetal mouse metanephros has been previously described in detail {Avner, E. D., Pediatr. Nephrol. 2:92 (1989); Avner, E. D., Kidney Int. 36: 960 (1989); Pediatr. nephrol. 4:372 (1990); Sweeney, W. E., J. Tiss. Cult. Meth. 13:163 (1991); Pugh, J. P., Kidney Int. 47:774 (1995)}. Briefly, intact methanephrae from Swiss Webster albino mouse embryos (E13±0.4 days gestation) or whole kidneys from day E-15 through P-14 are cut into 150 μm slices, cultured in chemically defined medium for 120 hours in a Trowell-type organ culture assembly at 36±0.5° C. and 95% humidity in a mixed air 5% CO₂ environment. The basal medium consists of equal volumes of Dublecco's modified Eagle's medium and Ham's F-12 medium supplemented with, for example, insulin (8.3×10⁻⁷ M), prostaglandin E₁ (7.1×10⁻⁸ M), selenium 6.8×10⁻⁹ M)), transferrin (6.2×10⁻⁸ M) and triiodothyronine (2×10⁻⁹ M). Growth factor(s) may also be included.

Supplemented medium consists of the basal medium to which either nothing (control) or the candidate compound has been added at concentrations in the range of 0.1 nM-1 μM. For example, Day 14 cystic (BPK) and control (Balb/C) explants may be cultured for 120 hours with a range of concentrations of the candidate compound. Tissue culture medium is replaced with freshly prepared medium every 24 hours.

Explant cultures are harvested at 120 hours and the degree of proximal tubular cyst formation is quantitated using a cystic index. This index was derived from basic light morphometric methods {Loud, A. V., Lab Invest. 50: 250 (1984)} and standardized as a tool for quantitation of cyst formation in organ culture systems {Pugh, J. P., Kidney Int. 47:774 (1995); Avner, E. D., Kidney Int. 28:447 (1985); Avner E. D., J. Lab. Clin. Med. 109:441 (1987)}. Following routine histologic preparation, 8 to 10 serial 3 μM sections of intact explants are graded, with an eyepiece micrometer, for cyst formation in Lotus tetragonolobus-positive tubular segments and Dolichos biflorus-positive tubular segments on a scale of 0 (no observable cysts) through 5 (multiple cysts larger than 0.20 mm). For each treatment group, a cystic index grading is repeated on multiple explants following 120 hours of incubation according to the following scale.

Cystic index

| | |
|---|---|
| 0 - no cyst observed | |
| 1 - single or multiple cysts | ≦0.05 mm |
| 2 - multiple cysts | >0.05 mm; ≦0.10 mm |
| 3 - multiple cysts | >0.10 mm; ≦0.15 mm |
| 4 - multiple cysts | >0.15 mm; ≦0.20 mm |
| 5 - multiple cysts | >0.20 mm |

The results will show whether the candidate compound reduces collecting tubule cyst lesions in a dose dependent manner, demonstrating the inhibition of cyst formation that is associated with polycystic kidney disease.

Following 120 hours of incubation, intact explants from control and treatment groups are assessed histologically. Tissue is fixed in 4.0% paraformaldehyde in phosphate buffer (pH 7.4) for 30 minutes at 4° C. Explants are then washed, dehydrated through graded acetone, and embedded in plastic embedding medium. Sections are cut at 3 μM on an ultramicrotome, mounted on glass slides, and stained with hematoxylin or segment specific lectins. Glomeruli are identified histologically as previously described {Sweeney W. E., J. Tiss. Cult. Meth. 13: 163 (1991)}. Proximal tubules are identified by staining with the lectin Lotus tetragonolobus (LTA) and collecting tubules are identified by staining with the lectin Dolichos biflorus (DBA) {Pugh J. P., Kidney. Int. 47: 774 (1995); Nauta, J., Pediatr. Nephrol. 7: 163 (1993)}. These studies will demonstrate whether the presence of candidate compound in the media alters collecting tubule cyst structures of the metanephroe.

In Vivo PKD Evaluation

Mice are divided into four groups; the first group are BPK (murine model of autosomal recessive PKD) animals treated with 0.25 mg of the candidate compound on days 7, 14, and 21 (e.g. given IP); the second group consist of untreated RPK controls; the third group are treated normal controls (same dosage regimen as above); and the fourth group are untreated normal controls. The pup litters of all groups are examined in late gestation for the presence of visibly cystic pups. Tissue is harvested from both groups on day 24. Kidneys and livers harvested from both litters can be fixed for 30 min in fresh 4% paraformaldehyde, rinsed, and dehydrated with graded acetone. The tissues are then left in immunobed for 40 hours, and then embedded.

Following dissection, the fresh kidneys are examined by gross analysis for kidney size and the presence of cysts. Total kidney volume is also evaluated in all four groups. Kidneys from the untreated BPK group are likely to be much larger than kidneys from the untreated control group. Kidneys from the test treated BPK group will be smaller than those of the untreated BPK group if the candidate compound affects cyst formation. The kidneys of the treated control group may have a slight decrease in kidney volume compared with the kidneys of the untreated control group.

Histologic evaluation of control non-cystic kidneys reveals effects on renal morphology in both test groups compared to untreated groups. Trichrome stains (for fibrosis) highlight the presence of fibrotic changes (collagen deposition). Control non-cystic kidneys show a well organized array of normal collecting tubular structures without cysts. Histologic sections of the day 24 untreated cystic kidneys reveal severe cystic disease with small islands of normal renal structures being compressed by expanding cystic lesions. Staining with DBA and LTA typically show that at this late stage of cystogenesis the lesions are almost entirely of collecting tubule origin. It is noteworthy that collecting tubule origin of cystic lesions is characteristic of human ARPKD.

In contrast to the untreated cystic kidneys, if the candidate compound is effective against cystic disease then kidneys derived from animals treated with the candidate compound will show a more moderate level of cystic disease, possibly with demonstrable normal collecting tubules amongst residual small proximal tubule cysts. In addition, trichrome staining (for fibrosis) of kidneys from a group treated with an effective candidate compound may show a reduction in collagen deposition and a well organized array of tubular structures.

Histologic staining of the liver may reveal a normal portal triad in control untreated animals. In contrast, livers from BPK animals are typically abnormal with multiple bile ducts and marked biliary epithelial hyperplasia. The livers from BPK animals that were treated with the candidate compound show a dramatic improvement in morphology with only slight biliary hyperplasia when the compound is effective.

The results obtained for representative compounds of the invention in these in vitro and in vivo experiments will indicate whether the compounds of the invention are candidates useful in treating or inhibiting polycystic kidney disease.

### Example 5

### LGL1 in a Rat Model of BPD

As discussed above, animal models of disease are useful to determine the involvement of LGL1 in normal function, in disease and in the treatment of abnormal function and disease. Useful models of lung disease may be in any animal including, for example an oxygen toxicity rat model (BPD-like symptoms) (Han, R. N. et al., (1996) Pediatr.Res. 39: 921-929), a baboon model of BPD (Coalson, J. J. et al., (1995) Am J Respir Crit Care Med 152:64) and models of congenital diaphragmatic hernia (CDH) in rabbits (see for example, Laberge J M et al., J Pediatr Surg (1999) 34:1183; Wu J. et al., Hum Reprod (2000) 15:2483-8) or sheep (Bratu I. et al., J Pediatr Surg (2001) 36:739).

Rat Model of Bronchopulmonary Dysplasia (BPD): Evaluation of LGL1 mRNA and lgl1 Protein

Deficiency in terminal lung differentiation due to premature birth is a leading cause of morbidity in the newborn period (respiratory distress syndrome), with sequelae that can extend into adulthood which is exhibited in the form of bronchopulmonary dysplasia (BPD). Although multiple pathophysiologic mechanisms are likely to contribute to BPD, it is currently believed that it is largely due to an arrest in alveolar development. LGL1 is maximally expressed in mesenchymal cells at rat fetal day 21, concordant with the onset of alveolar septation and the onset of augmented surfactant production. A rat model has been used to test the hypothesis that arrested alveolarization of postnatal injury may be associated with decreased expression of lgl1.

Methods

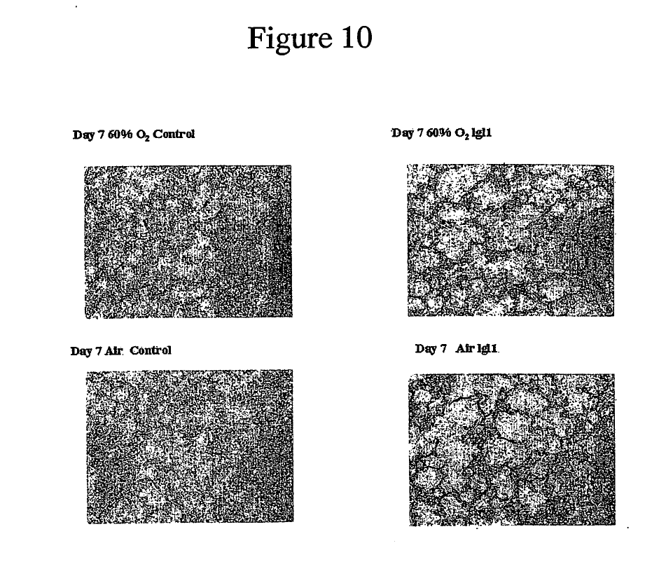

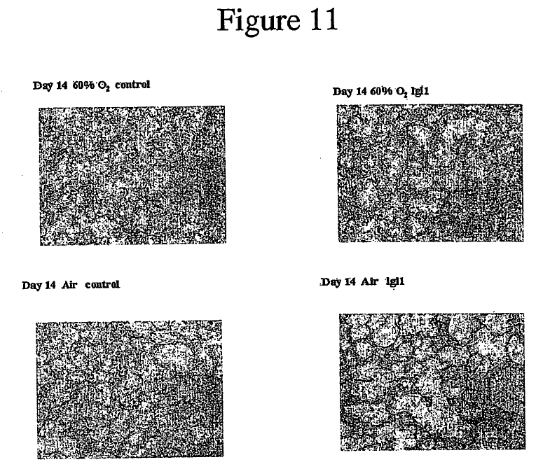

Newborn rats subject to hyperoxia model some of the oxidant/antioxidant interactions in the neonatal lung injury and dysplasia of BPD, as described by Han, R. N. et al., ((1996) Pediatr.Res. 39:921). In these studies, -neonatal rats were exposed to 60% oxygen for 7, 10 (data not shown) and 14 days to mimic BPD-like disease. Control litters received air (about 20% oxygen) for the same amount of time. Total RNA was isolated from the animals and used for Northern analysis using standard methodologies known to a worker skilled in the art (see also above sections and Kaplan et al (1999) Am. J. Physiol. 276 (Lung Cell. Mol. Physiol 20): L1027). Samples of lung tissue were also examined following immunohistochemical preparation as follows (see FIGS. 10 and 11).

Immunohistochemistry: Avidin-Biotin Complex (ABC) Method for Fixed Paraffin Sections

Lung tissue was fixed overnight at 4° C. in paraformaldehyde, embedded in paraffin and cut into 5 μm sections. Slides were baked for 1 hour at 60° C. Tissues were deparaffinized in xylene and rehydrated in decreasing concentrations of ethanol. Tissue sections were immunostained according to Hsu et al. ((1981) J Histochem Cytochem 29:577) with modifications. In short, antigen was retrieved by incubation in boiling 0.1M sodium citrate pH6. Washing with 0.3% Triton and PBS was followed by blocking of non-specific binding sites using 10% (v/v) Normal Goat Serum and 1% (w/v) BSA in PBS for 1 hour at room temperature. Standard procedures were used to raise polyclonal antibodies against the rat lgl1 peptide of SEQ ID NO: 14, and preliminary experiments determined optimal antibody concentrations. Rabbit anti-rat antiserum against lgl1 was used at 1:200 dilution and mouse antibody against vimentin and α-actin were used at 1:250 and 1:1000 respectively. Sections were incubated with primary antibodies overnight at 4° C., washed in PBS and then incubated with secondary antibody, either biotinylated anti rat IgG at 1:200 or biotinylated anti mouse IgG at 1:200, for 2 hours at room temperature. After washing with PBS, endogenous peroxidase activity was quenched with 1% (v/v) hydrogen peroxide in methanol for 20 minutes. Slides were then incubated with ABC for 1 hour at room temperature, TSA enhanced and developed using streptavidin HRP. Tissues were counterstained with 2% Methyl green and prepared for viewing.

Results

Immunohistochemistry also showed reduced amounts of the lgl1 protein in the BPD animals compared to controls in day 7 and day 14 animals (FIGS. 10 and 11 respectively). A diminution in lgl1 protein levels is also apparent between the day 7 and day 14 lungs of control animals, indicating that the amount of lgl1 protein diminishes with time after its peak expression at fetal day 21. A Northern Blot of total RNA isolated from the lung tissue and probed with a 1.7 kb LGL1 probe corresponding to nucleotides 1-1735 of SEQ ID NO: 1 (Kaplan et al (1999) ibid), also demonstrated decreased levels of LGL1 expression in BPD rats compared to controls at every time point (data not shown). These data support a role for LGL1 during the period of alveolarization in the neonatal rat and indicate that a deficiency of lgl1 may contribute to the arrested alveolar partitioning observed in BPD.

### Example 6

### Transgenic and Knockout Animal Models

Transgenic animals express an altered gene and/or have altered gene expression. Generally, the alterations to a naturally occurring gene can be modifications, deletions and substitutions. The altered gene generally should not fully encode the same gene as is native to the host animal, and its expression product should be altered to a minor or great degree, or absent altogether. However, it is conceivable that a more modestly modified gene will fall within the scope of the present invention, and animals that express a normal gene can also be made (see below).

Transgenic methodologies have been used to generate animal models demonstrating symptoms of disease (for example emphysema or polycystic kidney disease [Zhou, L., et al. (1996). Dev. Biol. 175:227; Nguyen, H., et al. (1996) Oncogene 12:2109; Hoyle, G. W., et al. (1999) Am J Pathol. 154:1763; Wert, S., et al. (1993) Dev. Biol. 156:426]). Animals carrying a “knockout” of the LGL1 gene or an LGL1 transgene are useful for the establishment of a nonhuman model for diseases involving both chronic lung disorders of pre-mature birth and acquired lung disease of later onset which are marked by defects in alveolar formation and/or destruction of alveoli in human lung.

Studies of LGL1 transgenic and knockout animals will yield information on the role of LGL1 expression, activity and mechanism of action in branching morphogenesis, alveolar development, and other aspects of lung maturation and function. Similarly, the role of LGL1 in cell events and interactions, and the development, maturation, diseases and disease treatments of other tissues, for example the kidney, brain, pancreas, liver, spleen and intestine may be studied in these LGL1 animal models.

Methods

In general, a knockout mouse can be generated by homologous recombination of a target DNA construct with the endogenous gene in the chromosome or using other techniques familiar to a worker skilled in the art. The DNA construct can be prepared from a genomic clone of LGL1 isolated from a genomic DNA library. The genes used for altering a target gene (if desired) may be obtained by a wide variety of techniques that include, but are not limited to, isolation from genomic sources, preparation of cDNAs from isolated mRNA templates, direct synthesis, or a combination thereof.

Any technique known in the art can be used to introduce a knockout construct or transgene into animals to produce the founder lines of transgenic animals. Such techniques include, but are not limited to pronuclear microinjection (Gordon et al., 1980, Proc. Natl. Acad. Sci. USA 77:7380; Gordon & Ruddle, 1981, Science 214: 1244-1246; U.S. Pat. No. 4,873,191 (Oct. 10, 1989) T. E. Wagner and P. C. Hoppe); retrovirus mediated gene transfer into germ lines (Van der Putten et al., 1985, Proc. Natl. Acad. Sci. USA 82: 6148-152); gene targeting in embryonic stem cells (Thompson et al., 1989, Cell 56: 313-321); electroporation of embryos (Lo, 1983, Mol. Cell. Biol. 3: 1803-1814); and sperm-mediated gene transfer (Lavitrano et al., 1989, Cell 57: 717-723); etc. For a review of such techniques, see Gordon, 1989, Transgenic Animals, Intl. Rev. Cytol. 115: 171-229, which is incorporated by reference herein in its entirety). Once the founder animals are produced, they can be bred, inbred, crossbred or outbred to produce colonies of transgenic or knockout animals, which may themselves be used, for example, for further breeding and/or directly for studies, for example on disease generation and treatment.

Transgenic Mice that Express LGL1

LGL1 expression has been targeted to the respiratory mesenchyme cells of transgenic mice using a Versican-LGL1 transgene. First, the entire coding region of rat LGL1 cDNA (nucleotides 86-1576 of SEQ ID NO: 1) was generated by RT-PCR using primers LGL1-5′and LGL1-FLAG. The carboxy terminus of the translated protein from this cDNA construct contains a FLAG epitope tag. The LGL1-flag tagged cDNA, a 1.51 kb (5′, 3′- EcoRI) fragment, was subcloned into the pCDNA 3.0 vector. Correct orientation of the insert was assessed by restriction enzyme digest using Cla I and Hind III, to generate a 1021 bp insert and 5889-bp vector fragments. This construct was then sequenced and transfected into HEK 293T cells. Western blot analysis with anti-flag m2 antibodies was used to verify generation of the lgl1-flag protein (FIG. 2).

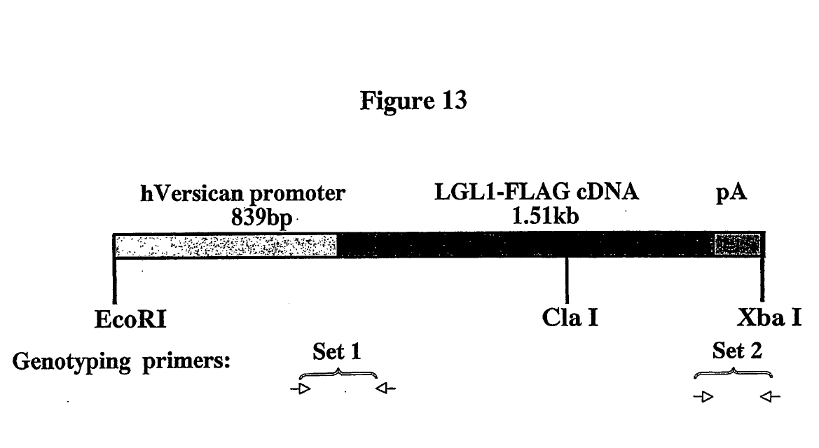

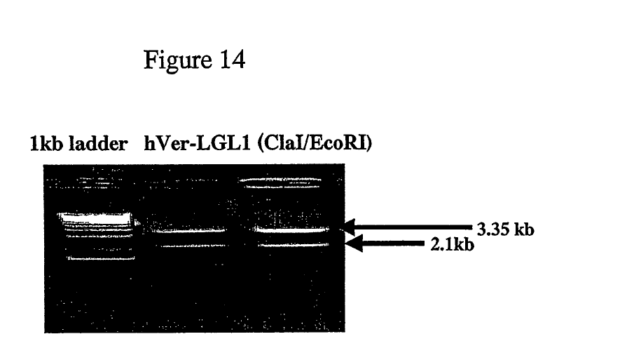

The versican-LGL1 transgene was then constructed by ligation of a 1.51 kb fragment of rat LGL1 cDNA with a C-terminus FLAG tag epitope into the Not I site of a vector containing the 839 bp-hVersican promoter region with a synthetic slice donor/acceptor site, and a polyadenylation signal sequence at the 3′end (FIG. 13) [Wert, S., et al. (1993) Dev. Biol. 156:426; Bignami, A., et al. (1993) J.Neurosci.Res. 34:97; Glasser, S. W., et al. (1991) Am.J.Physiol. 261:L349; Kishimoto, J., et al. (1999) Proc.Natl.Acad.Sci U.S.A 96:7336]. Proper orientation of the cDNA insert was again verified by restriction enzyme digest (FIG. 14). The plasmid DNA was propagated in E. coli DH5α using standard methods. The Versican-LGL1 transgene was then excised as an EcoR I-Xba I 4391 bp fragment and purified from low melting point gel electrophoresis using the Quiaex Extraction Kit (Quiagen Inc). Purified DNA was dialyzed into 5 mM Tris-HCl (pH. 7.4)/0.1 mM EDTA, in preparation for pronuclear microinjection.

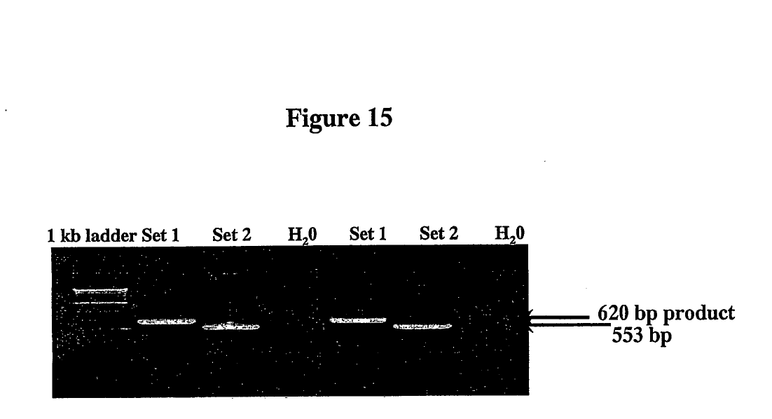

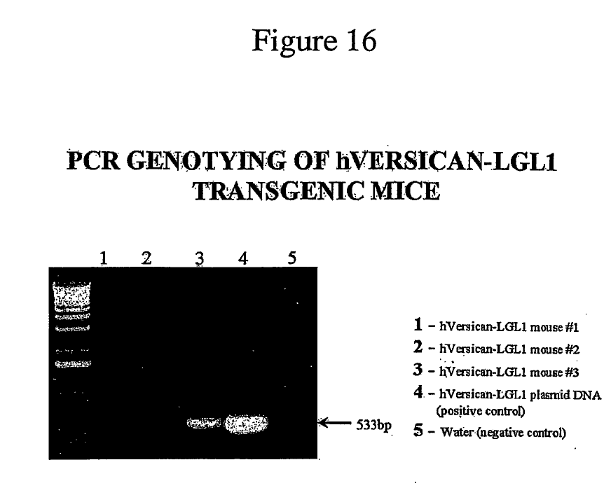

The purified, lineralized transgene construct was then injected into the pronuclei of FVB/N mouse oocytes using standard transgenic protocols of the Hospital for Sick Children (Toronto, Ontario, Canada). Offspring were tested for chromosomal integration of the transgene by PCR analysis of genornic DNA derived from the tail and/or Southern blot A PCR protocol has been designed specific for each transgene, where the 5′ primer anneals the hVersican promoter sequence and the 3′ primer anneals to rat LGL1 coding sequence (FIG. 15). The 478 bp SV40 small t-intron poly A sequence and the hVersican promoter/splice sites were used as probes for Southern blot genotyping of the Versican-lgl1 transgenic mice. FIG. 16 demonstrates the expression of the transgene by mouse #3.

LGL1 “Knock-Out” Mice

Preparation of an LGL1 construct: DNA constructs of LGL1 (SEQ ID NO: 1) were prepared using standard methodology known in the art. In order to identify a clone containing both the 5′ and 3′ ends (i.e. the complete sequence) of LGL1, six BAC clones containing the LGL1 transgene were analyzed. BAC clone No. 34304 was shown to contain the full-length LGL1 sequence. Comparison of a partial sequence of BAC 34304 with the ENSEMBL mouse genome sequence identified a gene on mouse chromosome 8 in the 119 Mb-120 Mb region.

Developing the targeting vector: The following procedure was used in order to produce an LGL1 knock out mouse model. It is well known that the ATG start codon is situated in exon 2 of LGL1. A restriction map of LGL1 revealed that a 13.7 kb EcoRI genomic fragment containing exon 2 has ideal flanking regions on both sides and has suitable digestion sites. Clones of this fragment were inserted into a simple vector using methods familiar to a worker skilled in the art.

To confirm the accuracy of the restriction map, a 600 bp probe corresponding to 10566-11185 bp of the LGL1 29034 bp genomic sequence, amplified by PCR was used to hybridize with completely digested BAC 34304. The digestion enzymes used were: BamHI, BglII, EcoRI, HincII, SacI and SmaI. The deduced sequence was confirmed using the 600 bp PCR probe which picked up strong band signals of the following sizes: BamHI-9.6 kb; BglII-6.8 kb; EcoRI-13.7 kb; HincII-7 kb+0.8 kb; SacI-6 kb; and SmaI-12 kb.

The 13.7 kb EcoRI fragment was then cloned into the simple vector, pQZ1BamHI, a 2.4 kb vector modified from pUC19 with the lacZ region deleted and the multiple cloning sites modified to EcoRI-BamHI-EcoRI for easy cloning. The vector was digested with EcoRI and treated with CIP to remove the phosphate group at the ends. BAC 34304 was digested with EcoRI and the 13.7 kb fragment recovered from a low-melting point agarose gel. The ligation product was electro-transferred into host DH10B cell and then plated on a selection plate. To select the colony with the correct ligation, colonies on the plate were transferred to a nylon membrane, fixed and probed with the 600 bp probe. Colonies with a positive signal were selected and plasmids extracted for restriction analysis

Choosing Suitable Vectors: Correctly ligated pQZ1-13.7 kb plasmids may be used for vector construction using methods known to a worker skilled in the art. For example, the pQZ1 BamH1 vector itself is a suitable targeting vector that has most of the necessary sequence. In order to use such a vector simply remove exon 2 of the LGL1 gene and insert a Neo gene. The pMC1 neo vector may also be used for such a purpose.

Evaluation of Different Enzymes Suitable for Vector Construction: For the right flanking region, the HincII site in pQZ1-13.7 kb may be used and for the left flanking region, either MluI, KpnI or HindIII may be used for cleaving the LGL1 gene. Since a large fragment of LGL1 has been cloned, the subcloning step is relatively straightforward. The first set of probes chosen to identify the cross-over have been prepared and purified.

Generation of Knockout Mice: The LGL1 construct may be introduced into an animal by any of a number of methods known to a worker skilled in the art. For example, the targeting vector may be introduced by homologous recombination into embryonic stem (ES) cells using standard techniques, as outlined by Lodish et al. ((2000), Molecular Cell Biology) or other techniques familiar to a worker skilled in the art. Briefly, ES cells containing the LGL1 construct (exon 2 deleted and Neo inserted) may be micro-injected into the cavity of a recipient mouse blastocyst. The blastocyst is then transferred into a surrogate pseudopregnant mouse and the resultant chimeric mice are mated to assess whether the mutation has incorporated into the germ line. Mice heterozygous for the knockout mutation will then be mated to produce homozygous knockout mice.

Investigations using Transgenic and Knockout Animals

Before experiments begin all transgenic and knockout animals are genotyped by PCR analysis of tail DNA using standard procedures. The impact of altered LGL1 expression or function on lung development, including alveolar septation may be determined, for example, by analysis of morphology, physiology and gene expression (see below). These studies may identify lgl1 protein itself as a potential therapeutic agent, and may also permit identification of other gene products of potential therapeutic utility.

Measuring Morphology Using Immunohistochemical Analyses: It is known that alveolar septation in the mouse occurs largely during the critical initial two weeks of post-natal life. Therefore histology of lung tissue isolated from wild type, transgenic heterozygote and homozygote mutant LGL1 mice may be compared at late gestational ages (e.g. 13.5, 15.5, 17.5 and 18.5 days (term)), and early post-natal ages (e.g. 1, 3, 7 and 14 days after birth). Alveolar development may be assessed at the light microscopic level, using hematoxylin and eosin stains and immunohistochemistry (IHC) performed to detect markers of epithelial (cytokeratin, CC10, Surfactant Protein C) and mesenchymal (vimentin, smooth muscle α-actin) cell types using standard mmunohistochemistry techniques familiar to a worker skilled in the art (see also methods outlined above).

Assessment of Lung Physiology: The maturity of pulmonary physiology may be monitored by assessing terminal epithelial cell differentiation at gestational days 17.5 and 18.5. For example, the method used by Oomen for analysis of fetal rat lung tissue development can be used in primary cultures of fetal mouse distal airway epithelial cells isolated from control and knockout LGL1 mice. Surfactant production is assessed by measuring the rates of incorporation of radiolabeled precursor into surfactant phospholipids and analysed using thin layer chromatography and Western blotting using surfactant protein A as described in Sweezey et al., ((1995) Pediatr Res 38:506). Trans-epithelial ion transport may be assessed, for example using Ussing chambers as described by Sweezey et al., ((1998) Am J Physiol. 1998 275:L103). Briefly, monolayer cultures of lung epithelial cells grown on permeable supports may be assessed for transepithelial electrical potential difference and short-circuit current, and for sensitivity to well-characterized antagonists and/or agonists of the epithelial sodium channel (ENaC) or chloride channels, including the cystic fibrosis transmembrane conductance regulator (CFTR).

Assessment of Post-natal Lung phenotype of Adult Mice: Pulmonary function tests may be performed on adult mice using methods adapted from human infant pulmonary function testing. Briefly, anesthetized, tracheotomized and paralyzed mice are placed in a plethysmographic box and ventilated using an infant ventilator. Changes in lung flow and volume are recorded using a pneumotachograph connected to a pressure transducer. Airway pressure signals are measured at the mouth. A box is used as a reservoir of negative pressure, generated and regulated from a wall source. Three computer-regulated solenoids are placed in the expiratory limb of the circuit. The first solenoid is activated at full inspiration to occlude the inspiratory/expiratory line of the ventilator. In order to generate a forced deflation, the second solenoid opens the circuit to the negative pressure box. For a relaxed deflation, the third solenoid opens the circuit to atmospheric pressure. Data acquisition is on-line, with computerized data storage and analysis.

In situ Hybridization Studies: Pulmonary vascular formation on days 17.5 and 18.5 may be assessed by in situ hybridization (ISH) using probes for the vascular-associated genes VEGF-R1 and VEGF-R2. Specific antibodies for vascular markers such as CD34, von Millebrand factor, and PECAM-1 in immunohistochemistry (IHC) may also be used in the assessment of pulmonary vascular formation in transgenic or knockout and control neonatal rats.

Genetic Analyses—Impact of altered LGL1 expression on downstream Gene Expression: Lung maturation may be monitored in transgenic and knockout mice by measuring gene expression using conventional methods known in the art. For example Northern analysis may be used to monitor the expression of a panel of developmentally regulated genes and/or markers of lung differentiation at 13.5, 15.5, 17.5 and 18.5 days of gestation and in neonatal animals. Examples of useful markers include surfactant proteins SP-A, SP-B and SP-C, CC10, TTF1, mineralocorticoid receptor (MR), glucocorticoid receptor (GR) and epithelial sodium channel (ENaC) subunits α, β and γ.

Alternatively, or in addition, cDNA microarrays may be productively employed, as hybridization to high density (e.g. Affymetrix) microarrays allows thousands of genes and expressed sequence tags to be monitored (see, for example, the microarray methodology used by R. Novak in Jaroslav P. et al., (2002) Genomics, 79:104). This approach may be used to identify altered gene expression resulting from altered LGL1 expression in transgenic and knockout animals. For example, differences in lung RNA expression in fetal and newborn lungs might be monitored in control and experimental animals at the series of time points indicated above (Kaplan et al., (2002) Chest 121(3 Suppl):90S). Gene products identified with significant alterations in expression can be further characterized, for example by monitoring mRNA and protein expression and localization during key fetal and neonatal periods by Northern and Western Blot analysis, in situ hybridization and immunohistochemistry, respectively. It is contemplated that such investigations using transgenic or knockout animals and complementary investigations using other suitable techniques (e.g. cell culture) will identify gene products downstream of lgl1 activities that are important to the developmental processes of fetal lung maturation, including branching morphogensis and alveolarization. The involvement of these gene products and of lgl1 may be further tested by determining whether lgl1 administration restores normal lung function, thereby suggesting a potential therapeutic role in treatment of disorders of lung development and later onset lung injury.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims. All cited patents, patent applications and publications referred to in this application are herein incorporated by reference in their entirety.

1. A nucleic acid molecule comprising a polynucleotide selected from the group consisting of:
(c) a polynucleotide with a sequence as set forth in SEQ ID NO: 1 or 3;
(d) a polynucleotide that encodes a protein with a sequence as set forth in SEQ ID NO: 2 or 4;
(e) a polynucleotide that is at least about 90% identical to the polynucleotide of (a) or (b);
(f) a polynucleotide fragment of the polynucleotide of (a), (b) or (c) which encodes a polypeptide fragment having at least one activity of lgl1;
(g) a polynucleotide capable of hybridizing under stringent conditions to the polynucleotide of (a), (b), (c) or (d).
(h) a polynucleotide capable of hybridizing under stringent conditions to a polynucleotide that is complementary to the polynucleotide of (a), (b), (c) or (d).

2. A nucleic acid homologue of the nucleic acid molecule of claim 1, wherein said nucleic acid homologue is produced by a process comprising:
(a) screening a genomic DNA library using as a probe a target sequence defined by the SEQ ID NO: 1, or fragments thereof;
(i) identifying members of said library which contain sequences that hybridize to said target sequence; and
(j) isolating an intact coding sequence from one or more of said members identified in step (b).

3. A nucleic acid homologue of the nucleic acid molecule of claim 1, wherein said nucleic acid molecule is produced by a process comprising:
(a) isolating mRNA, DNA, or cDNA from tissue;
(b) amplifying polynucleotides from said isolated mRNA, DNA, or cDNA, that contain a nucleotide sequence complementary to amplification primers, that are complementary to fragments of SEQ ID NO: 1 and designed to prime said amplification; and
(c) isolating said amplified sequences produced in step (b).

4. The nucleic acid molecule according to claim 1 (d), wherein the polynucleotide fragment consists of the coding region of SEQ ID NO: 1 or SEQ ID NO: 3.

5. The nucleic acid molecule of claim 1 or 4 wherein said nucleic acid molecule is operably linked to one or more expression control elements.

6. A vector comprising an isolated nucleic acid molecule of claim 1.

7. A host cell transformed to contain the nucleic acid molecule of any one of claims 1-5.

8. A host cell comprising the vector of claim 6.

9. A method for producing a host cell which expresses an lgl1 polypeptide comprising transfecting the host cell with the nucleic acid molecule of claim 5 or 6.

10. A host cell produced by the method of claim 9.

11. A method for producing a polypeptide comprising culturing a host cell transformed with the nucleic acid molecule of any one of claims 1-5 under conditions in which the protein encoded by said nucleic acid molecule is expressed.

12. A polypeptide produced by the method of claim 11.

13. A polypeptide selected from the group consisting of:
(a) a polypeptide comprising the amino acid sequence of SEQ ID NO: 2 or 4;
(b) a polypeptide comprising a fragment of at least 6 contiguous amino acids of-SEQ ID NO: 2 or 4;
(c) a polypeptide as in (a) or (b) comprising one or more conservative amino acid insertions, deletions or substitutions;
(k) a polypeptide comprising naturally occurring amino acid sequence variants of SEQ ID NO: 2 or 4; and
(l) a polypeptide comprising a fragment of lgl1.

14. An isolated antibody that binds to a polypeptide of either claim 12 or 13 or a fragment thereof.

15. An antibody of claim 14 wherein said antibody is a monoclonal or a polyclonal antibody.

16. A compound which specifically inhibits expression of the nucleic acid molecule of claim 1.

17. An antisense compound capable of blocking expression of a polypeptide of claim 13.

18. A nucleic acid molecule that modulates the expression of lgl1, wherein said nucleic acid molecule comprises a nucleotide sequence from the 5′ untranslated region of SEQ ID NO: 1 or 3, or a degenerate variant thereof.

19. A method of identifying an agent that modulates the expression of a nucleic acid molecule encoding the polypeptide of SEQ ID NO: 2 or 4 comprising:
(a) exposing cells which express the nucleic acid to the agent; and
(b) determining whether the agent modulates expression of said nucleic acid, thereby identifying an agent which modulates the expression of a nucleic acid encoding the polypeptide of SEQ ID NO: 2 or 4.

20. A method of modulating the expression of a nucleic acid molecule encoding the polypeptide of SEQ ID NO: 2 or 4 comprising using an effective amount of an agent which modulates the expression of a nucleic acid molecule encoding the polypeptide of SEQ ID NO: 2 or 4.

21. A method of identifying an agent that modulates at least one activity of the polypeptide of SEQ ID NO: 2 or 4 comprising:
(a) exposing cells which express the polypeptide to the agent; and
(b) determining whether the agent modulates at least one activity of said polypeptide, relative to control cells not exposed to said agent.

22. A method of modulating at least one activity of the polypeptide of SEQ ID NO: 2 or 4 comprising using an effective amount of an agent which modulates said activity of the polypeptide of SEQ ID NO: 2 or 4.

23. A method of identifying an agent that modulates one activity of the polypeptide of SEQ ID NO: 2 or 4 comprising:
(a) exposing cells which express the polypeptide to the agent; and
(b) determining whether the agent modulates one activity of said polypeptide, relative to control cells not exposed to said agent.

24. A method of modulating one activity of the polypeptide of SEQ ID NO: 2 or 4 in cells comprising administering an effective amount of an agent which modulates one activity of the polypeptide of SEQ ID NO: 2 or 4 to said cells.

25. An agent identified by the method of any one of claims 19, 21 or 23.

26. A method of identifying binding partners for the polypeptide of SEQ ID NO: 2 or 4 comprising:
(a) exposing said polypeptide to a potential binding partner; and
(b) determining whether the potential binding partner binds to said protein, thereby identifying binding partners for the polypeptide of SEQ ID NO: 2 or4.

27. A diagnostic process comprising determining, in a sample derived from a host organism, the presence, absence or amount of a nucleic acid molecule according to claim 1.

28. A diagnostic process comprising determining, in a sample derived from a host organism, the presence, absence or amount of a polypeptide according to claim 13.

29. The diagnostic process of claim 28 wherein an antibody is used to determine the presence, absence or amount of polypeptide.

30. A composition comprising a diluent and a polypeptide of claim 13.

31. A pharmaceutical composition comprising at least one agent of claim 25 and a pharmaceutically acceptable carrier.

32. The pharmaceutical composition of claim 31 further comprising an agent which modulates the onset or progression of lung disease.

33. A method of treating lung disease in an animal comprising administering an effective amount of the pharmaceutical composition of claim 31 to said animal.

34. A method of modulating lung disease in an animal by administering an effective amount of a nucleic acid expressing lgl1 or a fragment thereof to said animal.

35. A method of modulating airway branching in an animal comprising administering an effective amount of a nucleic acid expressing lgl1 or a fragment thereof to said animal.

36. A method of modulating abnormal alveolarization in an animal by administering an effective amount of a nucleic acid expressing lgl1 or a fragment thereof to said animal.

37. A method of modulating airway branching, abnormal alveolarization or lung disease in an animal comprising the steps of:
(a) transforming cells in vitro with a nucleic acid encoding lgl1 or a fragment thereof;
(b) selecting the cells transformed in step (a) that express lgl1 or a fragment thereof; and
(c) introducing the cells selected in step (b) into the animal, thereby modulating airway branching, abnormal alveolarization or lung disease in an animal.

38. A method of modulating lung disease in an animal by administering an effective amount of lgl1 or a fragment thereof to said animal.

39. A method of modulating airway branching in an animal comprising administering an effective amount of lgl1 or a fragment thereof to said animal.

40. A method of modulating abnormal alveolarization in an animal by administering an effective amount of lgl1 or a fragment thereof to said animal.

41. The method according to any one of claims 33, 34, 37 or 38, wherein said lung disease is bronchopulmonary dysplasia (BPD).

42. The method according to any one of claims 33, 34, 37 or 38, wherein said lung disease is emphysema.

43. The method according to claim 42, wherein said animal is a human with a deficiency of alpha-1-antitrypsin.

44. The method according to any one of claims 33, 34, 37 or 38, wherein said lung disease is New BPD.

45. The method according to any one of claims 33, 34, 37 or 38, wherein said lung disease is chronic obstructive pulmonary disease (COPD).

46. The method according to any one of claims 33, 34, 37 or 38, wherein said lung disease is congenital diaphragmatic hernia (CDH).

47. The method according to any one of claims 33, 34, 37 or 38, wherein said lung disease comprises chronic bronchial infection.

48. The method according to any one of claims 34-40, wherein said lgl1 or fragment thereof is a recombinant polypeptide.

49. The method according to any one of claims 34-40, wherein said animal is a mammal.

50. The method according to claim 48, wherein said mammal is a human.

51. A pharmaceutical composition comprising a nucleic acid molecule according to claim 1 and a pharmaceutically acceptable carrier or diluent.

52. A pharmaceutical composition comprising the polypeptide according to claim 13 and a pharmaceutically acceptable carrier or diluent.

53. A use of a nucleic acid molecule according to claim 1 to prepare a medicament for use in the treatment of a lung disease or disorder.

54. A use of a polypeptide according to claim 1 to prepare a medicament for use in the treatment of a lung disease or disorder.
